(19) United States
(12) Patent Application Publication
(10) Pub. No.: US 2011/0003013 A1
(43) Pub. Date: Jan. 6, 2011

(54) METHOD OF INCREASING METABOLISM

(75) Inventor: Jenny Gunton, New South Wales (AU)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: GARVAN INSTITUTE OF MEDICAL RESEARCH

(21) Appl. No.: 12/811,340

(22) PCT Filed: Jan. 5, 2009
(86) PCT No.: PCT/AU09/00009

§ 371 Date: Sep. 15, 2010

(30) Foreign Application Priority Data
Jan. 4, 2008 (AU) 2008900039

(51) Int. Cl. A61K 31/4196; A61K 38/17; A61K 31/195; A61K 31/16; A61K 33/24; A61K 31/4439; A61K 31/444; A61K 31/4412; A61K 31/426; A61K 31/706; A61P 3/00; A61P 3/04; A61P 3/06

(52) U.S. Cl. 424/646; 514/383; 514/4.8; 514/5.4; 514/566; 514/616; 514/342; 514/335; 514/348; 514/365; 514/27

(57) ABSTRACT

The present invention provides a method for increasing metabolism and/or energy expenditure in a subject, e.g., to treat or prevent obesity and/or a related condition and/or to reduce adiposity, the method comprising increasing the level and/or activity of Hypoxia Induced Factor 1α (HIF-1α) in a cell, tissue or organ of the subject, thereby increasing metabolism in the subject. The present invention also provides a method for increasing metabolism in a subject, the method comprising administering an iron chelating agent to the subject, thereby increasing metabolism in the subject.

### RELATED APPLICATION DATA

This application claims priority from Australian Provisional Patent Application No. 2008900039, the contents of which is incorporated by reference.

### FIELD OF THE INVENTION

The present invention relates to a method for increasing metabolism or metabolic rate in a subject, e.g., to treat or prevent obesity or a related condition and/or to reduce adiposity in a subject.

### BACKGROUND OF THE INVENTION

Obesity is a common clinical problem in most developed nations and is also rapidly becoming a major health concern in developing nations. The incidence of obesity has increased dramatically throughout the world, most notably over the last 3 decades. By the year 2000, a total of 38.8 million American adults or 30% of the population of that country were classified as obese (i.e., having a body mass index score of at least 30 kg/m²) (Mokdad et al., JAMA 286:1195-1200, 2001). Obesity is associated with or thought to cause a number of diseases or disorders, and estimates attribute approximately 280,000 deaths each year in the United States to obesity related disorders (The Merck Manual of Diagnosis & Therapy, Beers & Brakow, 17th edition, Published by Merck Research Labs, Section 1, Chapter 5, Nutritional Disorders, Obesity (1999).

Obesity is a result of the long-term imbalance between overall energy intake and total energy expenditure (EE) (comprising resting EE, EE of activity and the thermic effects of feeding (Segal J Clin Nut. 40: 995-1000, 1984)). Fat is the main reservoir for storage of surplus calories. An imbalance between resting energy intake and energy expenditure whereby more energy is taken in than is expended results in an increased level of fat in a subject. Increases in fat storage in a subject can lead to a subject being overweight or obese. Such an increase in fat storage can occur independently of adipogenesis, i.e., the production of new fat cells. Furthermore, many complications of obesity (as discussed below) result from improper storage of fat in a subject, e.g., in the liver.

Obesity is a risk factor for developing many obesity-related complications, from non-fatal debilitating conditions, such as, for example, osteoarthritis and respiratory disorders, to life-threatening chronic disorders, such as, for example, hypertension, type 2 diabetes, stroke, cardiovascular disease, some forms of cancer and stroke. As the number of subjects that are obese is increasing (in the US alone the incidence of obesity increased one third in the last decade), the need to develop new and effective strategies in controlling obesity and obesity-related complications is becoming increasingly important. Upper body or truncal obesity is the strongest risk factor known for diabetes mellitus type 2, and is a strong risk factor for cardiovascular disease. Obesity is also a recognized risk factor for hypertension, atherosclerosis, congestive heart failure, stroke, gallbladder disease, osteoarthritis, sleep apnoea, reproductive disorders such as polycystic ovarian syndrome, cancers of the breast, prostate, and colon, and increased incidence of complications of general anaesthesia (see, e.g., Kopelman, Nature 404, 635-43, 2000). It reduces life span and carries a serious risk of co-morbidities as described above, as well as disorders such as infections, varicose veins, acanthosis nigricans, eczema, exercise intolerance, insulin resistance, hypertension hypercholesterolemia, cholelithiasis, orthopaedic injury, and thromboembolic disease (Rissanen et al., Brit. Med. J 301, 835-837, 1990). Obesity is also a risk factor for the group of conditions called insulin resistance syndrome, or “Syndrome X”.

Despite the high prevalence of obesity and increased weight and many advances in our understanding of how it develops, current therapeutic strategies have persistently failed to achieve long-term success (Crowley et al., Nat. Rev. Drug Disc. 1: 276-286, 2002). Present pharmacological interventions typically induce a weight loss of between five and fifteen kilograms. However, of the subjects that do lose weight, approximately 90 to 95 percent subsequently regain their lost weight (Rosenbaum et al., N. Engl. J. Med. 337:396-407 1997).

The most commonly used strategies currently used for treating obesity and related disorders include dietary restriction, incremental increases in physical activity, pharmacological and surgical approaches. In adults, long term weight loss is exceptional using conservative interventions.

There are also few therapeutic drugs approved by the FDA for the long term treatment of obesity. One of these compounds, orlistat, is a pancreatic lipase inhibitor that acts by blocking fat absorption into the body. However, the use of this drug is also accompanied by the unpleasant side effects of the passage of undigested fat from the body.

Another drug commonly used for the treatment of obesity is sibutramine, an appetite suppressant. Sibutramine is a β-phenethylamine that selectively inhibits the reuptake of noradrenaline and serotonin in the brain. Unfortunately, the use of sibutramine is also associated with elevated blood pressure and increased heart rate. As a result of these side effects dosage of sibutramine is limited to a level that is below the most efficacious dose.

Compounds for the short term treatment of obesity include, appetite suppressants, such as amphetamine derivatives. However, these compounds are highly addictive. Furthermore, subjects respond differently to these weight-loss medications, with some losing more weight than others and some not losing any weight whatsoever.

To date, few pharmaceutical strategies have focussed on increasing the caloric metabolism, i.e., metabolic rate, of a subject to thereby reduce their bodyweight and/or treat or prevent obesity.

As will be apparent to the skilled artisan from the foregoing, there is a need for effective therapeutics and/or prophylactics for obesity and/or weight gain and/or obesity, preferably therapeutics and/or prophylactics that increase metabolism and/or energy expenditure of a subject and/or decrease food intake of a subject. Preferred therapeutic and/or prophylactic compounds will have reduced side effects that cause discomfort to the majority of subjects using the compound and/or that are useful for treating a diverse population of subjects.

### SUMMARY OF THE INVENTION

In work leading up to the present invention, the inventors sought to characterize the role for the protein Hypoxia Induced Factor 1α (HIF-1α) in obesity, and to determine whether or not increasing the level and/or activity of this protein in a subject reduces or prevents weight gain and/or adiposity and/or obesity and, if so, the mechanism of action of this protein.

The inventors have found that increasing levels of HIF-1α in a subject or a tissue or organ thereof results in increased metabolism in the subject. This increased metabolism is associated with or causative of reduced weight gain or adiposity in a subject. The inventors also found that HIF-1 α-mediated increased metabolic rate was associated with or causative of treatment or prevention of obesity in two accepted animal models of obesity in humans, i.e., mice fed on a high-fat diet and ob/ob mice.

As exemplified herein in one preferred form of the invention, the inventors have increased metabolic rate by administering a chelating compound, to a subject e.g. an iron chelating compound, such as a compound that bind to iron and prevents its use or uptake by a cell. For example, the inventors have demonstrated that a substituted 3,5-diphenyl-1,2,4-triazole iron chelator such as desferrioxamine (DFO) is capable of increasing metabolism in a subject and reducing or preventing weight gain or adiposity in the subject and/or treating or preventing obesity in the subject. Treatment with this compound was associated with increased HIF-1 α levels in the subject. Without being bound by theory or mode of action, the inventors consider that the chelating compound inhibits a protein that mediates or induces or enhances HIF-1α protein degradation, e.g., Von Hippel-Lindau (VHL) protein (pVHL) or a HIF-1α specific prolyl-4 hydroxylase e.g., Prolyl Hydroxylase Domain-Containing Protein (PHD) 1 (HPH3, EGLN2), PHD2 (HPH2, EGLN1), or PHD3 (HPH1, EGLN3). For example, PHD1, PHD2 and PHD3 require an iron molecule for their biological activity in hydroxylating a proline residue in HIF-1 α (e.g., Pro-402 and Pro-564), and the hydroxylated HIF-1α is then ubiquinated by the ubiquitin ligase complex comprising pVHL. Accordingly, an iron chelating agent reduces ubiquitination of HIF-1α and increases intracellular levels of this protein.

The inventors have also found that by administering a compound that increases the level and/or activity of Hypoxia Induced Factor 1α (HIF-1α) in a cell, tissue or organ of the subject and/or administering a chelating agent, the level of expression of genes involved in fat metabolism is also increased.

Based on the inventors' findings, e.g., as described herein, the present invention provides a method for increasing metabolism and/or energy expenditure in a subject, said method comprising increasing the level and/or activity of Hypoxia Induced Factor 1α (HIF-1α) in a cell, tissue or organ of the subject, thereby increasing metabolism in the subject.

In one embodiment, the increased metabolism is or includes increased fat metabolism.

In one embodiment, the increase in metabolism in the subject reduces adiposity in the subject and/or prevents an increase in adiposity in the subject and/or treats or prevents obesity or associated insulin resistance in the subject. For example, HIF-1α levels are increased in a subject suffering from or at risk of developing adiposity or obesity and/or associated insulin resistance. Optionally, the increase in metabolism is associated with altered appetite or caloric intake, preferably reduced appetite or caloric intake.

In one embodiment, the increased metabolism in the subject is independent of the activity level of the subject.

In one embodiment, the increased metabolism is associated with or caused by increased expression of genes involved in fat metabolism and/or is associated with or caused by increased fat metabolism.

The present invention also provides a method for preventing or treating obesity and/or associated insulin resistance and/or increasing metabolism and/or reducing adiposity in a subject, the method comprising increasing the level and/or activity of Hypoxia Induced Factor 1α (HIF-1α) in a cell, tissue or organ of the subject, thereby preventing or treating obesity and/or adiposity and/or associated insulin resistance and/or increasing metabolism in the subject.

In one embodiment of the invention, the cells of the subject are adipocytes or skeletal muscle cells or cells of the nervous system involved in regulation of energy intake and energy expenditure or the tissue is fat or skeletal muscle or neural tissue.

In a preferred embodiment of the invention, the level and/or activity of HIF-1α is increased by administering to the subject a compound that increases the level or activity of HIF-1α in a cell, tissue or organ thereof. Preferably, the compound increases stability and/or reduces degradation of HIF-1α in a cell, tissue or organ of the subject thereby resulting in an increased levels and/or activity of said protein. Preferably, the compound reduces degradation of HIF-1α by inhibiting or completely inhibiting or preventing activity of a protein that mediates degradation of HIF-1α, e.g., a Von Hippel-Lindau (VHL) protein (pVHL) or a HIF-1α specific prolyl-4 hydroxylase e.g., Prolyl Hydroxylase Domain-Containing Protein (PHD) 1 (HPH3, EGLN2), PHD2 (HPH2, EGLN1), or PHD3 (HPH1, EGLN3).

In one embodiment, the inhibitor of a protein that mediates degradation of HIF-1α reduces expression (e.g., transcription and/or translation) of said protein. An exemplary inhibitor is an antisense nucleic acid, a ribozyme, a PNA, an interfering RNA, a siRNA, a microRNA or an antibody. Preferably, the inhibitor is a siRNA.

In an additional or alternative embodiment of the invention, the level or activity of HIF-1α is increased by administering to the subject a chelating agent. Such an increase may be by direct or indirect means. Preferably, the chelating agent is an iron chelating agent (syn. iron chelator). Suitable iron chelators will be apparent to the skilled artisan and/or described herein. Exemplary iron chelators include a bidentate iron chelator or a tridentate iron chelator or a higher order multidentate (e.g., hexadentate) iron chelator or a non-naturally occurring iron chelator. The iron chelator is preferably selected individually or collectively from the group consisting of deferasirox (DFS; 4-[3,5-Bis (2-hydroxyphenyl)-1H-1,2,4-triazol-1-yl]-benzoic acid), desferrioxamine (DFO; N-(5-C3-L (5 aminopentyl)hydroxycarbamoyl)-propionamido)pentyl)-3(5-(N-hydroxyactoamido)-pentyl)carbamoyl)-propionhydroxamic acid), Feralex G (2-deoxy-2-(N-carbamoylmethyl-[N′-2′-methyl-3′-hydroxypyridin-4′-one])-D-glucopyranose), pyridoxal isonicotinyl hydrazone (PIH), GT56-252 (4,5-dihydro-2-(2,4-dihydroxyphenyl)-4-methylthiazole-4-carboxylic acid), desferrithiocin/DFT (4,5-dihydro-2-(3′-hydroxypyridin-2′-yl)-4-methylthiazole-4-carboxylic acid, ICL670 (4-[3,5-bis(2-hydroxyphenyl)-[1,2,4]triazol-1-yl]benzoic acid, HBED (N,N′-bis(o-hydroxybenzyl)ethylenediamine-N,N′-diacetic acid), ferrioxamine, trihydroxamic acid, CP94, EDTA, desferrioxamine hydroxamic acids, deferoxamine B (DFOB) as the methanesulfonate salt also known as desferrioxamine B mesylate (DFOM), desferal from Novartis (previously Ciba-Giegy), apoferritin, CDTA (trans-1,2-diaminocyclohexane-N,N,N′,N′-tetraacetic acid), and DTPA (diethylenetriamine-N,N,N′,N″,N″-penta-acetic acid), deferiprone (1,2 dimethyl-3-hydroxypyridin-4-one), a cobaltous ion, a non-crystal form of any of the foregoing, a crystal form of any of the foregoing, a salt of any of the foregoing, a derivative of any of the foregoing and mixtures thereof.

In one example of the present invention, the iron chelator is a tridentate iron chelator, e.g., a 3,5-diphenyl-1,2,4-triazole, e.g., in its free acid form or a salt thereof or a crystalline form thereof. Preferably, the compound is 4-[3,5-Bis (2-hydroxyphenyl)-1H-1,2,4-triazol-1-yl]-benzoic acid, DFS) or a salt thereof.

In a preferred embodiment, the iron chelator is deferasirox (DFS) or desferrioxamine (DFO). Preferably, the iron chelator is DFS.

In another embodiment of the invention, the level of HIF-1α is increased by administering to the subject a compound that increases HIF-1α expression, e.g., a HIF-1α polypeptide or an active fragment thereof or a derivative or analogue thereof, or a polynucleotide encoding the HIF-1α polypeptide or an active fragment thereof.

In one embodiment of the invention, the polynucleotide is a vector encoding a HIF-1α polypeptide or active fragment thereof. Preferably the vector is a viral vector.

In a further preferred embodiment of the invention, the vector is within a cell. Preferably, the cell is an adipocyte and/or a skeletal muscle cell and/or a cell of the nervous system involved in regulation of energy intake and energy expenditure and/or a hepatocyte and/or a cell capable of differentiating into an adipocyte and/or a skeletal muscle cell and/or a cell of the nervous system involved in regulation of energy intake and energy expenditure and/or a hepatocyte.

In one embodiment, the cell is autologous to the subject to whom it is to be administered.

The present invention also provides a method for increasing metabolism and/or energy expenditure in a subject, the method comprising administering a chelating agent to the subject. Preferably, the chelating agent is an iron chelating agent. Suitable chelating agents are described herein and are to be taken to apply mutatis mutandis to the present embodiment of the invention. Preferably, the increase in metabolism and/or energy expenditure in the subject reduces adiposity in the subject and/or prevents an increase in adiposity in the subject and/or treats or prevents obesity associated insulin resistance. Preferably, the method increases metabolism in a subject.

The present invention also provides a method for preventing or treating obesity and/or associated insulin resistance and/or increasing metabolism and/or reducing adiposity in a subject, the method comprising administering a chelating agent to the subject. Preferably, the chelating agent is an iron chelating agent. Suitable chelating agents are described herein and are to be taken to apply mutatis mutandis to the present embodiment of the invention.

The present invention also provides a method for increasing metabolism and/or energy expenditure in a subject, the method comprising administering deferasirox (DFS; 4-[3,5-Bis (2-hydroxyphenyl)-1H-1,2,4-triazol-1-yl]-benzoic acid) or a salt thereof to the subject. Preferably, the increase in metabolism and/or energy expenditure in the subject reduces adiposity in the subject and/or prevents an increase in adiposity in the subject and/or treats or prevents obesity associated insulin resistance. Preferably, the method increases metabolism in the subject.

The present invention also provides a method for preventing or treating obesity and/or associated insulin resistance and/or increasing metabolism and/or reducing adiposity in a subject, the method comprising administering deferasirox (DFS; 4-[3,5-Bis (2-hydroxyphenyl)-1H-1,2,4-triazol-1-yl]-benzoic acid) or a salt thereof to the subject.

The methods of the invention can be performed on a range of different subjects. Preferably, the subject is a mammal. More preferably, the subject is human.

In a preferred embodiment, the compound or agent is administered in an effective amount, preferably a therapeutically effective amount and/or a prophylactically effective amount.

In one embodiment, the compound or agent is administered a plurality of times to a subject. For example, the compound is administered on a regular basis. Alternatively, or in addition the method of the present invention additionally comprises determining adiposity or an estimate thereof (e.g., body mass index) and/or weight and/or metabolic rate and/or HIF-1α level and/or pVHL in a subject and administering a compound that increases HIF-1α activity and/or levels if required.

In one embodiment, the compound or agent is administered in the form of a pharmaceutical composition additionally comprising a pharmaceutically acceptable carrier and/or diluent. Optionally, the pharmaceutical composition comprises an additional component, such as a compound that reduces appetite and/or increases metabolism and/or prevents digestion of a lipid (e.g., a lipase inhibitor).

In one embodiment, the method of the present invention also comprises determining a subject at risk of developing obesity and/or excessive adiposity and/or insufficient metabolism. Preferably, the subject has reduced HIF-1α and/or increased pVHL compared to a normal and/or healthy subject.

The present invention also provides for use of a compound that increases HIF-1α levels and/or activity in a cell, tissue or organ of a subject to increase metabolism and/or energy expenditure in the subject. Preferably, the increase in metabolism in the subject reduces adiposity in the subject and/or prevents an increase in adiposity in the subject and/or treats or prevents obesity associated insulin resistance. The present invention also provides for use of a compound that increases HIF-1 α levels and/or activity in a cell, tissue or organ of a subject to treat or prevent obesity and/or associated insulin resistance and/or to increase metabolism and/or to reduce adiposity. Similarly, the present invention also provides a compound that increases HIF-1α levels and/or activity in a cell, tissue or organ of a subject for use in increasing metabolism and/or energy expenditure in the subject. Preferably, the increase in metabolism in the subject reduces adiposity in the subject and/or prevents an increase in adiposity in the subject and/or treats or prevents obesity associated insulin resistance. The present invention also provides a compound that increases HIF-1 α levels and/or activity in a cell, tissue or organ of a subject for use in treating or preventing obesity and/or associated insulin resistance and/or to increase metabolism and/or to reduce adiposity. Suitable compounds are described herein and are to be taken to apply mutatis mutandis to the present embodiments of the invention. Preferably, the compound increases metabolism in a subject.

Furthermore, the present invention provides for use of a compound that increases HIF-1α levels and/or activity in a cell, tissue or organ of a subject in the manufacture of a medicament to increase metabolism and/or energy expenditure in a subject. Preferably, the increase in metabolism in the subject reduces adiposity in the subject and/or prevents an increase in adiposity in the subject and/or treats or prevents obesity associated insulin resistance. The present invention also provides for use of a compound that increases HIF-1α levels and/or activity in a cell, tissue or organ of a subject in the manufacture of a medicament to treat or prevent obesity and/or associated insulin resistance and/or to increase metabolism and/or to reduce adiposity. Suitable compounds are described herein and are to be taken to apply mutatis mutandis to the present embodiment of the invention. Preferably the compound increases metabolism in a subject.

The present invention also provides a kit or article of manufacture comprising a compound that increases HIF-1α levels and/or activity in a cell, tissue or organ of a subject packaged with instructions to use the compound to increase metabolism and/or energy expenditure in a subject, preferably to increase metabolism in a subject. The instructions may indicate use of the compound to increase metabolism and/or energy expenditure for the purposes of weight loss and/or to treat obesity. The present invention also provides a kit or article of manufacture comprising a compound that increases HIF-1α levels and/or activity in a cell, tissue or organ of a subject packaged with instructions to use the compound to treat or prevent obesity and/or associated insulin resistance and/or to increase metabolism and/or to reduce adiposity. Preferably, said kit or article of manufacture is used in a method of the present invention. Suitable compounds are described herein and are to be taken to apply mutatis mutandis to the present embodiment of the invention.

As will be apparent, preferred features and characteristics of one embodiment of the present invention are applicable mutatis mutandis to other embodiments of the invention unless explicitly stated otherwise.

### BRIEF DESCRIPTION OF THE ACCOMPANYING DRAWINGS

FIG. 1 is a copy of a photographic representation showing HIF-1 α protein is present in a range of normal tissues. Tissues were isolated from wild-type mice and immediately snap-frozen in liquid nitrogen. HIF-1α protein was detectable following immunoprecipitation in liver, muscle, kidney, whole brain, pancreas and in Min6 cells which were used as a positive control.

FIGS. 2A-2D are a graphical representations showing DFS treatment decreases weight gain in mice on a high fat diet. Mice fed a high fat diet (60% calories from fat) more weight on average than mice fed a high fat diet with DFS. *, p<0.05; **, p<0.05 and ***, p<0.005.

FIG. 3 is a graphical representation showing oxygen consumption (VO₂) in control mice (boxes) and DFS treated mice (diamonds) in the initial week of high fat diet consumption and DFS treatment. There is no significant difference in VO₂ between the groups. The period in which mice are housed in the light is indicated. *, p<0.05.

FIG. 4 is a graphical representation showing oxygen consumption (VO₂) in control mice (boxes) and DFS treated mice (diamonds) after 8 weeks of high fat diet consumption and DFS treatment. Mice treated with DFS consume significantly more oxygen than control mice, suggesting improved whole body metabolism. The period in which mice are housed in the light is indicated. *, p<0.05.

FIG. 5 is a graphical representation showing oxygen consumption (VO₂) in control mice (boxes) and DFS treated mice (diamonds) after 25 weeks of high fat diet consumption and DFS treatment. Mice treated with DFS consume significantly more oxygen than control mice, suggesting improved whole body metabolism. The period in which mice are housed in the light is indicated. *, p<0.05.

FIG. 6 is a graphical representation showing respiratory exchange ratio (RER) in control mice (boxes) and DFS treated mice (diamonds). RER is calculated as (VCO₂/VO₂). Mice treated with DFS have significantly reduced RER than control mice. Results indicate that DFS treated mice use more fat as an energy source (e.g., it is their predominant energy source) than control mice (carbohydrate preferred energy source).

FIGS. 7A and 7B are graphical representations showing the weight of visceral adipose tissue (VAT), white adipose tissue (WAT) and brown adipose tissue (BAT) in DFS treated (grey bars) and control (black bars) mice. Visceral and white adipose tissues of DFS treated mice are significantly lighter than those of control animals, whereas BAT is unchanged, indicating DFS treatment results in reduced white adipose levels. *, p<0.05.

FIG. 8 is a graphical representation showing the weight of food consumed per mouse when adjusted for the weight of the mouse. DFS treated mice, black boxes, control mice, grey boxes. DFS mice consumed significantly more food per gram of body weight than control mice, despite having a lower body weight. *, p<0.05.

FIG. 9 is a graphical representation showing energy expenditure (EE) or heat production in control mice (boxes) and DFS treated mice (diamonds). Results indicate that DFS treated mice have significantly higher energy expenditure over a day than control mice. *, p<0.05.

FIG. 10 is a graphical representation showing mean fasting insulin levels in DFS treated mice (black boxes) and control mice (grey boxes). DFS mice have significantly lower fasting insulin levels than control mice. **, p<0.005.

FIG. 11 is a graphical representation showing glucose stimulated insulin secretion (GSIS) at week 7 in DFS treated mice (diamonds) and control mice (squares). Food intake studies were carried out at weeks 0, 4, 8 and 25. Indirect calorimetry was performed using the Oxymax System (Columbus Instruments, Columbus, Ohio) at weeks 0, 4, 8 and 25. Measurements were taken over a 12-hour light cycle and a 12-hour dark cycle.

FIG. 12 is a graphical representation showing blood glucose levels assessed at random times over 9 weeks of high fat diet in DFS treated (diamond) and control (square) mice. At several time points, DFS treated mice have significantly lower blood glucose levels than control mice. *, p<0.05.

FIG. 13A is a graphical representation showing results of glucose tolerance tests in mice treated with DFS (diamond) or control mice (square) following five weeks of high fat diet. DFS treated mice have significantly improved glucose tolerance compared with control mice.

FIG. 13B is a graphical representation showing results of glucose tolerance tests in mice treated with DFS (diamond) or control mice (square) following 21 weeks of high fat diet. DFS treated mice have significantly improved insulin tolerance compared with control mice.

FIG. 14 is a graphical representation showing results of insulin tolerance tests in mice treated with DFS (diamond) or control mice (square) following five weeks of high fat diet. DFS treated mice have significantly improved insulin tolerance compared with control mice.

FIG. 15 is a copy of photographic representations showing Western blots to detect the level of HIF-1α relative to levels of β-tubulin in mice treated with DFS compared to a control mouse. DFS treated mice have higher levels of HIF-1α than the control.

FIG. 16 is a series of graphical representations showing the level of expression of genes involved in metabolism in subjects treated with DFS (DFS) and control subjects (Con). HSL: hormone sensitive lipase, LPL: lipoprotein lipase, IRS-1: insulin receptor matrix-1. Results show that DFS treatment results in increased expression of genes involved in lipid metabolism, e.g., HSL and LPL and in insulin signalling, e.g., IRS-1.

FIG. 17A is a graphical representation showing haemoglobin levels in DFS treated mice and control mice (as indicated). No significant difference in haemoglobin levels is detected.

FIG. 17B is a graphical representation showing white blood counts for DFS treated mice and control mice (as indicated). No significant difference in white blood counts levels is detected.

FIG. 18A is a graphical representation showing serum alanine transaminase levels in DFS treated mice and control mice (as indicated). No significant difference in alanine transaminase levels is detected. However, DFS treated mice have a tendency to have lower alanine transaminase levels, indicating improved liver function.

FIG. 18B is a graphical representation showing serum aspartate aminotransferase levels in DFS treated mice and control mice (as indicated). No significant difference in aspartate aminotransferase levels is detected. However, DFS treated mice have a tendency to have lower aspartate aminotransferase levels, indicating improved liver function.

FIG. 19 is a graphical representation showing serum iron levels in DFS treated mice and control mice (as indicated). No significant difference in serum iron levels is detected.

FIG. 20 is a graphical representation showing weight of wild-type (wt) mice treated with DFS (triangles), control wt mice (hatch), ob/ob mice treated with DFS (diamond) and control ob/ob mice. All mice are fed on a chow (low fat) diet).

FIG. 21 is a graphical representation showing the amount of weight gained by ob/ob mice treated with DFS or control ob/ob mice over an eight week period. DFS treated ob/ob mice gained significantly less weight than control ob/ob mice. All mice were fed on a chow diet (low fat diet). ***, p<0.0005.

### KEY TO SEQUENCE LISTING

SEQ ID NO: 1=Homo sapiens HIF-1α protein isoform 1 [accession no. NP_{—}001521]
SEQ ID NO: 2=Homo sapiens HIF-1α protein isoform 2 [accession no. NP_{—}851397]
SEQ ID NO: 3=Mus musculus HIF-1α protein [accession no. NP_{—}034561]
SEQ ID NO: 4=Rattus norvegicus HIF-1α protein [accession no. NP_{—}077335]
SEQ ID NO: 5=Homo sapiens HIF-1α cDNA variant 1 [accession no. NM_{—}001530]
SEQ ID NO: 6=Homo sapiens HIF-1α cDNA variant 2 [accession no. NM_{—}181054]
SEQ ID NO: 7=Mus musculus HIF-1α cDNA [accession no. NM_{—}010431]
SEQ ID NO: 8=Rattus norvegicus HIF-1α cDNA [accession no. NM_{—}024359]
SEQ ID NO: 9=Homo sapiens VHL protein isoform 1 [accession no. NP_{—}000542]
SEQ ID NO: 10=Homo sapiens VHL protein isoform 2 [accession no. NP_{—}937799]
SEQ ID NO: 11=Mus musculus VHL protein [accession no. NP_{—}033533]
SEQ ID NO: 12=Rattus norvegicus VHL protein [accession no. NP_{—}434688]
SEQ ID NO: 13=Homo sapiens VHL cDNA variant 1 [accession no. NM_{—}000551]
SEQ ID NO: 14=Homo sapiens VHL cDNA variant 2 [accession no. NM_{—}198156]
SEQ ID NO: 15=Mus musculus VHL cDNA [accession no. NM_{—}009507]
SEQ ID NO: 16=Rattus norvegicus VHL cDNA [accession no. NM_{—}052801]
SEQ ID NO: 17=VHL siRNA
SEQ ID NO: 18=VHL siRNA
SEQ ID NO: 19=VHL siRNA
SEQ ID NO: 20=VHL siRNA
SEQ ID NO: 21=Homo sapiens PHD1 protein
SEQ ID NO: 22=Homo sapiens PHD2 protein
SEQ ID NO: 23=Homo sapiens PHD3 protein
SEQ ID NO: 24=Homo sapiens PHD1 cDNA
SEQ ID NO: 25=Homo sapiens PHD2 cDNA
SEQ ID NO: 26=Homo sapiens PHD3 cDNA

### DETAILED DESCRIPTION OF THE PREFERRED EMBODIMENTS

### General

This specification contains nucleotide and amino acid sequence information prepared using PatentIn Version 3.4. Each nucleotide sequence is identified in the sequence listing by the numeric indicator <210> followed by the sequence identifier (e.g. <210>1, <210>2, <210>3, etc). The length and type of sequence (DNA, protein (PRT), etc), and source organism for each nucleotide sequence, are indicated by information provided in the numeric indicator fields <211>, <212> and <213>, respectively. Nucleotide sequences referred to in the specification are defined by the term “SEQ ID NO:”, followed by the sequence identifier (e.g. SEQ ID NO: 1 refers to the sequence in the sequence listing designated as <400>1).

The designation of nucleotide residues referred to herein are those recommended by the IUPAC-IUB Biochemical Nomenclature Commission, wherein A represents Adenine, C represents Cytosine, G represents Guanine, T represents thymine, Y represents a pyrimidine residue, R represents a purine residue, M represents Adenine or Cytosine, K represents Guanine or Thymine, S represents Guanine or Cytosine, W represents Adenine or Thymine, H represents a nucleotide other than Guanine, B represents a nucleotide other than Adenine, V represents a nucleotide other than Thymine, D represents a nucleotide other than Cytosine and N represents any nucleotide residue.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

The present invention is not to be limited in scope by the specific embodiments described herein, which are intended for the purpose of exemplification only. Functionally-equivalent products, compositions and methods are clearly within the scope of the invention, as described herein.

Any embodiment herein directed to increasing HIF-1 α expression or activity or administering a compound that modulates HIF-1 α expression or activity shall be taken to apply mutatis mutandis to the administration of an iron chelating agent or a compound of formula (I) or administration of DFS or administration of 4-[3,5-Bis (2-hydroxyphenyl)-1H-1,2,4-triazol-1-yl]-benzoic acid or administration of [4-[(3Z,5E)-3,5-bis(6-oxo-1-cyclohexa-2,4-dienylidene)-1,2,4-triazolidin-1-yl]benzoic acid irrespective of any mechanism of action.

Embodiments set forth herein shall be taken to apply mutatis mutandis to a method for increasing lipid metabolism and/or for increasing expression of a gene involved in lipid metabolism.

### Selected Definitions

As used herein, the term “adiposity” shall be taken to mean the amount of fat, preferably white fat within an individual. This term is not limited to the absolute amount of fat within an individual, and encompasses estimates or surrogate readings of the amount of fat in an individual. For example, adiposity may be measured by near infra-red analysis or dual X-ray absorptiometry (DXA) analysis, skinfold measurement, bioelectrical impedence measurement, arm X-ray fat analysis, magnetic resonance imaging, BMI, girth measurement or bodyweight measurement. A compound that reduces adiposity reduces the absolute or estimated amount of adiposity, e.g., as determined by a method listed previously. Such a compound is useful for reducing adiposity in an obese subject or in a subject that is not yet obese but wishes to reduce adiposity, e.g., an overweight subject or a competitive athlete (e.g., a bodybuilder). Any embodiment herein directed to reducing adiposity shall be taken to apply mutatis mutandis to preventing an increase in adiposity.

A “chelating agent” refers to a substance, compound, mixture, or formulation capable of having an affinity for iron, copper or other transition metal and which is capable of binding iron or copper or any other transition metal in vitro or in vivo. Without being bound by any theory or mode of action, when used in the context of the present invention, the chelating agent is useful in chelating/binding ferrous iron or copper or other transition metal and/or decreasing oxidative stress by acting as a transition metal sequestrant and/or antioxidant.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein the term “derived from” shall be taken to indicate that a specified integer may be obtained from a particular source albeit not necessarily directly from that source.

As used herein, the term “effective amount” shall be taken to mean a sufficient quantity of a compound that increases HIF-1α expression and/or activity to increase HIF-1α expression and/or activity in a cell, tissue or organ of a subject compared to the level in the cell, tissue or organ prior to administration and/or compared to a cell, tissue or organ from a subject of the same species to which the compound has not been administered. Preferably, the term “effective amount” means a sufficient quantity of a compound that increases HIF-1α expression and/or activity to increase metabolism and/or energy expenditure in a subject or cell, tissue or organ thereof and, optionally, to reduce body fat content and/or insulin resistance and/or increase metabolism in a subject compared to the subject prior to treatment or compared to a subject of the same species living under similar conditions (e.g., consuming a similar diet and/or undertaking a similar level of exercise) to which the compound has not been administered. The skilled artisan will be aware that such an amount will vary depending on, for example, the specific compound(s) administered and/or the particular subject and/or the type or severity or level of obesity or adiposity. Accordingly, this term is not to be construed to limit the invention to a specific quantity, e.g., weight or amount of a compound, rather the present invention encompasses any amount of the compound sufficient to achieve the stated result in a subject.

The term “effective amount” in the context of an iron chelator shall also be taken to mean a sufficient quantity of a compound to chelate an increased amount of iron in a cell, tissue or organ of a subject compared to the level of chelated iron in the cell, tissue or organ prior to administration and/or compared to a cell, tissue or organ from a subject of the same species to which the compound has not been administered. In one example, the term “effective amount” in the context of an iron chelator to increase metabolism and/or energy expenditure in a subject or cell, tissue or organ thereof, and, optionally, to reduce body fat content and/or insulin resistance and/or increase metabolism in a subject compared to the subject prior to treatment or compared to a subject of the same species living under similar conditions (e.g., consuming a similar diet and/or undertaking a similar level of exercise) to which the compound has not been administered. The skilled artisan will be aware that such an amount will vary depending on, for example, the specific compound(s) administered and/or the particular subject. Accordingly, this term is not to be construed to limit the invention to a specific quantity, e.g., weight or amount of a compound, rather the present invention encompasses any amount of the compound sufficient to achieve the stated result in a subject.

As used herein, the term “therapeutically effective amount” shall be taken to mean a sufficient quantity of a compound to reduce or inhibit one or more symptoms of a clinical condition associated with or caused by reduced metabolism and, optionally, obesity and/or increased adiposity and/or insulin resistance to a level that is below that observed and accepted as clinically diagnostic of that condition. For example, a therapeutically effective amount of a compound that increases HIF-1α expression and/or activity and/or of an iron chelator may reduce the body mass index (BMI) of a subject to less than 25 kg/m².

As used herein, the term “prophylactically effective amount” shall be taken to mean a sufficient quantity of a compound to prevent or inhibit or delay the onset of one or more detectable symptoms of a clinical condition associated with or caused reduced metabolism, and, optionally obesity and/or increased adiposity and/or insulin resistance and/or reduced metabolism. For example, a prophylactically effective amount of a compound that increases HIF-1α expression and/or activity or of an iron chelator may prevent BMI in a subject from exceeding 25 kg/m².

By “individually” is meant that the invention encompasses the recited compound or agent or groups of compounds and/or agents separately, and that, notwithstanding that individual compounds and/or agents or groups of compounds and/or agents may not be separately listed herein the accompanying claims may define such compound or agent or groups of compounds and/or agents separately and divisibly from each other.

By “collectively” is meant that the invention encompasses any number or combination of the recited compounds and/or agents or groups of compounds and/or agents, and that, notwithstanding that such numbers or combinations of compounds and/or agents or groups of compounds and/or agents may not be specifically listed herein the accompanying claims may define such combinations or sub-combinations separately and divisibly from any other combination of compounds and/or agents or groups of compounds and/or agents.

As used herein, the term “iron chelating agent” or “iron chelator” is intended to mean a compound that binds iron between two or more separate binding sites so as to form a chelate ring or rings. An iron chelating agent bound or complexed with iron is referred to herein as an iron chelate. An iron chelating agent can be bidentate (or didentate), which binds iron using two separate binding sites. Iron chelating agents of the invention also can be tridentate, tetradentate or higher order multidentate iron chelation agents binding iron with three, four or more separate binding sites, respectively. Iron chelating compounds of the invention include chelation compounds that can bind to all oxidation states of iron including, for example, iron (—II) state, iron (−1) state, iron (0) state, iron (I) state, iron (II) state (ferrous), iron (III) state (ferric), iron (IV) state (ferryl) and/or iron (V). Iron chelation therapy refers to the use of an iron chelator to bind with iron in vivo to form an iron chelate so that the iron loses its adverse physiological activity, e.g., ability to facilitate degradation of HIF-1α.

By “insulin resistance” it is meant a state in which a given level of insulin produces a less than normal biological effect (for example, uptake of glucose). Insulin resistance is prevalent in obese individuals.

As used herein “HIF-1” is characterised as a DNA-binding protein which binds to a region in the regulatory, preferably in the enhancer region, of a structural gene having the HIF-1 binding motif. Included among the structural genes which can be activated by HIF-1 are erythropoietin (EPO), vascular endothelial growth factor (VEGF), and glycolytic gene transcription in cells subjected to hypoxia. HIF-1 is composed of subunits HIF-1α and an isoform of HIF-1β. In addition to having domains which allow for their mutual association in forming HIF-1, the α and β subunits of HIF-1 both contain DNA-binding domains. The α subunit is uniquely present in HIF-1, whereas the β subunit (ARNT) is a component of at least two other transcription factors.

The term “HIF-1 α” means the alpha subunit of the HIF-1 dimeric protein. For the purposes of nomenclature only and not limitation, sequences of Human HIF-1α are set forth in SEQ ID NOs: 1 and 2, the sequences of murine HIF-1α are set forth in SEQ ID NOs: 3 and 4.

By “HIF-1α activity” is meant any activity mediated by a HIF-1 α protein, e.g., solus or in the context of the HIF-1 dimeric protein. This term encompasses the activity of HIF-1α in mediated gene expression, e.g., expression of stem cell factor (SCF), vascular endothelial growth factor (VEGF) Erythropoietin (Epo), Lactate Dehydrogenase-A (LDHA), Endothelin-1 (ET1), transferrin, transferrin receptor, Flk1, Fms-Related Tyrosine Kinase-1 (FLT1), Platelet-Derived Growth Factor-Beta (PDGF-Beta) or basic Fibroblast Growth Factor (bFGF). This term also encompasses nuclear translocation of a HIF-1α protein.

By “HIF-1α expression level” and grammatical equivalents shall be taken to mean the level of HIF-1 α mRNA and/or protein. Preferably, the term “HIF-1 α expression level” shall be taken to mean; the level of HIF-1 α protein. Methods for assessing the level of a mRNA in a cell will be apparent to the skilled artisan and include, for example, Northern blotting and quantitative PCR. Methods for assessing the level of a protein in a cell will be apparent to the skilled artisan and include, for example, Western blotting, enzyme-linked immunosorbent assay (ELISA), fluorescence-linked immunosorbent assay (FLISA) and radioimmunoassay. Suitable methods are described in more detail in, for example, J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The terms “metabolic rate” or “metabolism” are used interchangeably and shall be taken to mean the ability of a subject to utilize dietary intake for immediate energy needs, rather than store such dietary intake as body fat. This term shall be taken to encompass basal metabolic rate. Methods for determining metabolic rate or metabolism will be apparent to the skilled artisan and include methods involving either direct or indirect calorimetry, e.g., as described herein. Estimates of basal metabolic rate include The Harris-Benedict formula:

Men: BMR=66+(13.7×wt in kg)+(5×ht in cm)−(6.8×age in years)

Women: BMR=655+(9.6×wt in kg)+(1.8×ht in cm)−(4.7×age in years)

And Katch-McArdle formula:

BMR (men and women)=370+(21.6×lean mass in kg)

In one embodiment, increased metabolism” or “increased metabolic rate” shall be understood to mean increased metabolism of fat in a subject or a cell, tissue or organ thereof. This may be determined, for example, by determining the level of fat in a subject (e.g., repeatedly over time) and/or determining the level of a protein involved in fat metabolism or a transcript encoding same in a cell, tissue, organ or body fluid of a subject.

As used herein, the term “normal or healthy individual” or “normal or healthy subject” shall be taken to mean an individual or subject that does not suffer from obesity and/or increased adiposity and/or insulin resistance and/or reduced metabolism as assessed by any method known in the art and/or described herein

The term “obesity” refers to an individual who has a body mass index (BMI) of 25 kg/m² or more due to excess adipose tissue. Obesity can also be defined on the basis of body fat content: greater than 25% body fat content for a male or more than 30% body fat content for a female.

The term “body mass index” or “BMI” refers to a weight to height ratio measurement that estimates whether an individual's weight is appropriate for their height. As used herein, an individual's BMI is calculated as follows: BMI=body weight in kilograms divided by the square of the height in meters.

As used herein, the terms “treating”, “treat” or “treatment” include administering a therapeutically effective amount of an inhibitor(s) and/or agent(s) described herein sufficient to reduce or eliminate at least one symptom of the specified disease or condition.

As used herein, the terms “preventing”, “prevent” or “prevention” include administering a therapeutically effective amount of an inhibitor(s) and/or agent(s) described herein sufficient to stop or hinder the development of at least one symptom of the specified disease or condition.

The terms “polypeptide” and “protein” are generally used interchangeably and refer to a single polypeptide chain which may or may not be modified by addition of non-amino acid groups. It would be understood that such polypeptide chains may associate with other polypeptides or proteins or other molecules such as co-factors. The terms “proteins” and “polypeptides” as used herein also include variants, mutants, modifications, analogous and/or derivatives of the polypeptides of the invention as described herein.

The term “recombinant” in the context of a polypeptide refers to the polypeptide when produced by a cell, or in a cell-free expression system, in an altered amount or at an altered rate compared to its native state. In one embodiment, the cell is a cell that does not naturally produce the polypeptide. However, the cell may be a cell which comprises a non-endogenous gene that causes an altered, preferably increased, amount of the polypeptide to be produced. A recombinant polypeptide of the invention includes polypeptides which have not been separated from other components of the transgenic (recombinant) cell, or cell-free expression system, in which it is produced, and polypeptides produced in such cells or cell-free systems which are subsequently purified away from at least some other components.

By “substantially purified polypeptide” or “purified” we mean a polypeptide that has been separated from one or more lipids, nucleic acids, other polypeptides, or other contaminating molecules with which it is associated in its native state. It is preferred that the substantially purified polypeptide is at least 60% free, more preferably at least 75% free, and more preferably at least 90% or 05% or 97% or 98% or 99% free from other components with which it is naturally associated.

As used herein a “biologically active fragment” is a portion of a polypeptide of the invention which maintains a defined activity of the full-length polypeptide, namely be able to promote weight loss and/or reduce insulin resistance in an obese subject. In one embodiment, the biologically active fragment contains one and preferably both of the transactivation domains of HIF-1α. By “transactivation domains of HIF-1α” it is meant the NH₂-terminal transactivation domain (amino acids 531-575) and the COOH-terminal transactivation domain (amino acids 786-826) of HIF-1α that interact with general transcription machinery to activate transcription from promoters of HIF-1α. target genes. Biologically active fragments can be any size as long as they maintain the defined activity. In this respect, the biological activity of the fragment may be enhanced or reduced compared to that of the native form of HIF-1α. Preferably, biologically active fragments are at least 100, more preferably at least 200, and even more preferably at least 350 amino acids in length.

By an “isolated polynucleotide”, including DNA, RNA, or a combination of these, single or double stranded, in the sense or antisense orientation or a combination of both, dsRNA or otherwise, we mean a polynucleotide which is at least partially separated from the polynucleotide sequences with which it is associated or linked in its native state. Preferably, the isolated polynucleotide is at least 60% free, preferably at least 75% free, and most preferably at least 90% free from other components with which they are naturally associated. Furthermore, the term “polynucleotide” is used interchangeably herein with the term “nucleic acid”.

The term “exogenous” in the context of a polynucleotide refers to the polynucleotide when present in a cell, or in a cell-free expression system, in an altered amount compared to its native state. In one embodiment, the cell is a cell that does not naturally comprise the polynucleotide. However, the cell may be a cell which comprises a non-endogenous polynucleotide resulting in an altered, preferably increased, amount of production of the encoded polypeptide. An exogenous polynucleotide of the invention includes polynucleotides which have not been separated from other components of the transgenic (recombinant) cell, or cell-free expression system, in which it is present, and polynucleotides produced in such cells or cell-free systems which are subsequently purified away from at least some other components.

### General Techniques

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant protein, cell culture, and immunological techniques utilised in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

### HIF-1α Polypeptides and Polynucleotides

In some embodiments of the present invention, the methods of the present invention involve increasing the level and/or activity of HIF-1α in cells of the subject. Preferably the cells of the subject are adipocytes or skeletal muscle cells or cells of the nervous system involved in regulation of energy intake and energy expenditure.

In one embodiment, the methods of the invention involve administering to the subject a HIF-1α polypeptide or an active fragment thereof or a derivative or analogue thereof, or a polynucleotide encoding HIF-1α polypeptide or an active fragment thereof.

The HIF-1α polypeptide can be a substantially purified, or a recombinant polypeptide.

Preferably, the HIF-1α polypeptide comprises a sequence which shares at least 75% identity with a sequence as shown in any one of SEQ ID NOS: 1 to 4.

The % identity of a polypeptide can be determined by GAP (Needleman and Wunsch, 1970) analysis (GCG program) with a gap creation penalty=5, and a gap extension penalty=0.3. The query sequence is at least 25 amino acids in length, and the GAP analysis aligns the two sequences over a region of at least 25 amino acids. More preferably, the query sequence is at least 50 amino acids in length, and the GAP analysis aligns the two sequences over a region of at least 50 amino acids. More preferably, the query sequence is at least 100 amino acids in length and the GAP analysis aligns the two sequences over a region of at least 100 amino acids. Even more preferably, the query sequence is at least 250 amino acids in length and the GAP analysis aligns the two sequences over a region of at least 250 amino acids. Even more preferably, the GAP analysis aligns the two sequences over their entire length.

With regard to a defined polypeptide, it will be appreciated that % identity figures higher than those provided above will encompass preferred embodiments. Thus, where applicable, in light of the minimum % identity figures, it is preferred that the polypeptide comprises an amino acid sequence which is at least 75%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99% identical to the relevant nominated SEQ ID NO.

Amino acid sequence mutants of the polypeptides of the present invention can be prepared by introducing appropriate nucleotide changes into a nucleic acid of the present invention, or by in vitro synthesis of the desired polypeptide. Such mutants include, for example, deletions, insertions or substitutions of residues within the amino acid sequence. A combination of deletion, insertion and substitution can be made to arrive at the final construct, provided that the final polypeptide product possesses the desired characteristics.

Mutant (altered) polypeptides can be prepared using any technique known in the art. For example, a polynucleotide of the invention can be subjected to in vitro mutagenesis. Such in vitro mutagenesis techniques may include sub-cloning the polynucleotide into a suitable vector, transforming the vector into a “mutator” strain such as the E. coli XL-1 red (Stratagene) and propagating the transformed bacteria for a suitable number of generations. In another example, the polynucleotides of the invention are subjected to DNA shuffling techniques as broadly described by Harayama (1998) or using a mutation inducing PCR method. Products derived from mutated/altered DNA can readily be screened using techniques described herein to determine if they are able to confer enhanced weight loss and/or reduction in insulin resistance in an obese subject.

In designing amino acid sequence mutants, the location of the mutation site and the nature of the mutation will depend on characteristic(s) to be modified. The sites for mutation can be modified individually or in series, for example, by (1) substituting first with conservative amino acid choices and then with more radical selections depending upon the results achieved, (2) deleting the target residue, or (3) inserting other residues adjacent to the located site.

Amino acid sequence deletions generally range from about 1 to 15 residues, more preferably about 1 to 10 residues and typically about 1 to 5 contiguous residues.

Substitution mutants have at least one amino acid residue in the polypeptide molecule removed and a different residue inserted in its place. The sites of greatest interest for substitutional mutagenesis include sites identified as important for function. Other sites of interest are those in which particular residues obtained from various strains or species are identical. These positions may be important for biological activity. These sites, especially those falling within a sequence of at least three other identically conserved sites, are preferably substituted in a relatively conservative manner. Such conservative substitutions are shown in Table 1 under the heading of “exemplary substitutions”.

Furthermore, if desired, unnatural amino acids or chemical amino acid analogues can be introduced as a substitution or addition into the polypeptides of the present invention to produce an analogue of the protein. Such amino acids include, but are not limited to, the D-isomers of the common amino acids, 2,4-diaminobutyric acid, α-amino isobutyric acid, 4-aminobutyric acid, 2-aminobutyric acid, 6-amino hexanoic acid, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogues in general.

Also included within the scope of the invention are polypeptides of the present invention which are differentially modified during or after synthesis, for example, by biotinylation, benzylation, glycosylation, acetylation, phosphorylation, amidation, derivatization by known protecting/blocking groups, proteolytic cleavage, linkage to an antibody molecule or other cellular ligand, etc to thereby produce an analogue of the protein. These modifications may serve to increase the stability and/or bioactivity of the polypeptide of the invention.

TABLE 1
Exemplary Substitutions

| Original | Exemplary |
|---|---|
| Residue | Substitutions |
| Ala (A) | val; leu; ile; gly |
| Arg (R) | lys |
| Asn (N) | gln; his |
| Asp (D) | glu |
| Cys (C) | ser |
| Gln (Q) | asn; his |
| Glu (E) | asp |
| Gly (G) | pro, ala |
| His (H) | asn; gln |
| Ile (I) | leu; val; ala |
| Leu (L) | ile; val; met; ala; phe |
| Lys (K) | arg |
| Met (M) | leu; phe |
| Phe (F) | leu; val; ala |
| Pro (P) | gly |
| Ser (S) | thr |
| Thr (T) | ser |
| Trp (W) | tyr |
| Tyr (Y) | trp; phe |
| Val (V) | ile; leu; met; phe; ala |

The present invention also encompasses use of a derivative of a polypeptide as described herein in any embodiment. Such a derivative includes a polypeptide conjugated to another compound, e.g., polyethylene glycol (PEG) essentially as described by Tsubery et al., J. Biol. Chem. 279 (37) pp. 38118-38124. Without being bound by any theory or mode of action, such a derivative provides for extended or longer half-life of the protein moiety in circulation. Alternatively, the protein is conjugated to a nanoparticle such as hydrogel, PLGA or a protein which has the capacity to bind to an abundant serum protein such as human serum albumin.

Polypeptides of the present invention can be produced in a variety of ways, including production and recovery of natural polypeptides, production and recovery of recombinant polypeptides, and chemical synthesis of the polypeptides. In one embodiment, an isolated polypeptide of the present invention is produced by culturing a cell capable of expressing the polypeptide under conditions effective to produce the polypeptide, and recovering the polypeptide. Effective culture conditions include, but are not limited to, effective media, bioreactor, temperature, pH and oxygen conditions that permit polypeptide production. An effective medium refers to any medium in which a cell is cultured to produce a polypeptide of the present invention. Such medium typically comprises an aqueous medium having assimilable carbon, nitrogen and phosphate sources, and appropriate salts, minerals, metals and other nutrients, such as vitamins. Cells can be cultured in conventional fermentation bioreactors, shake flasks, test tubes, microtiter dishes, and petri plates. Culturing can be carried out at a temperature, pH and oxygen content appropriate for a recombinant cell. Such culturing conditions are within the expertise of one of ordinary skill in the art.

Alternatively, a protein or peptide or derivative or analogue is synthesized, e.g., using known techniques of solid phase, liquid phase, or peptide condensation, or any combination thereof. Amino acids used for peptide synthesis may be standard Boc (Nα-amino protected Nα-t-butyloxycarbonyl)amino acid resin with the deprotecting, neutralization, coupling and wash protocols of the original solid phase procedure of Merrifield, J. Am. Chem. Soc., 85:2149-2154, 1963, or the base-labile Nα-amino protected 9-fluorenylmethoxycarbonyl (Fmoc) amino acids described by Carpino and Han, J. Org. Chem., 37:3403-3409, 1972. Both Fmoc and Boc Nα-amino protected amino acids can be obtained from various commercial sources, such as, for example, Fluka, Bachem, Advanced Chemtech, Sigma, Cambridge Research Biochemical, Bachem, or Peninsula Labs.

Generally, chemical synthesis methods comprise the sequential addition of one or more amino acids to a growing peptide chain. Normally, either the amino or carboxyl group of the first amino acid is protected by a suitable protecting group. The protected or derivatized amino acid can then be either attached to an inert solid support or utilized in solution by adding the next amino acid in the sequence having the complementary (amino or carboxyl) group suitably protected, under conditions that allow for the formation of an amide linkage. The protecting group is then removed from the newly added amino acid residue and the next amino acid (suitably protected) is then added, and so forth. After the desired amino acids have been linked in the proper sequence, any remaining protecting groups (and any solid support, if solid phase synthesis techniques are used) are removed sequentially or concurrently, to render the final polypeptide. By simple modification of this general procedure, it is possible to add more than one amino acid at a time to a growing chain, for example, by coupling (under conditions which do notracemize chiral centers) a protected tripeptide with a properly protected dipeptide to form, after deprotection, a pentapeptide. See, e.g., J. M. Stewart and J. D. Young, Solid Phase Peptide Synthesis (Pierce Chemical Co., Rockford, Ill. 1984) and G. Barany and R. B. Merrifield, The Peptides: Analysis, Synthesis, Biology, editors E. Gross and J. Meienhofer, Vol. 2, (Academic Press, New York, 1980), pp. 3-254, for solid phase peptide synthesis techniques; and M. Bodansky, Principles of Peptide Synthesis, (Springer-Verlag, Berlin 1984) and E. Gross and J. Meienhofer, Eds., The Peptides: Analysis. Synthesis. Biology, Vol. 1, for classical solution synthesis. These methods are suitable for synthesis of a polypeptide of the present invention or an active fragment thereof or a derivative or an analogue thereof.

A polypeptide as described herein according to any embodiment can also be chemically prepared by other methods such as by the method of simultaneous multiple peptide synthesis. See, e.g., Houghten Proc. Natl. Acad. Sci. USA 82: 5131-5135, 1985 or U.S. Pat. No. 4,631,211.

In another embodiment, the methods of the invention involve administration of a polynucleotide encoding HIF-1α or an active fragment thereof. The HIF-1α polynucleotide can be an isolated or exogenous polynucleotide. Preferably, the HIF-1α polynucleotide comprises a sequence which shares at least 75% identity with a sequence as shown in any one of SEQ ID NOS: 5 to 8.

The % identity of a polynucleotide can be determined by GAP (Needleman and Wunsch, 1970) analysis (GCG program) with a gap creation penalty=5, and a gap extension penalty=0.3. Unless stated otherwise, the query sequence is at least 45 nucleotides in length, and the GAP analysis aligns the two sequences over a region of at least 45 nucleotides. Preferably, the query sequence is at least 150 nucleotides in length, and the GAP analysis aligns the two sequences over a region of at least 150 nucleotides. More preferably, the query sequence is at least 300 nucleotides in length and the GAP analysis aligns the two sequences over a region of at least 300 nucleotides. Even more preferably, the GAP analysis aligns the two sequences over their entire length.

With regard to the defined polynucleotides, it will be appreciated that % identity figures higher than those provided above will encompass preferred embodiments. Thus, where applicable, in light of the minimum % identity figures, it is preferred that a polynucleotide of the invention comprises a sequence which is at least 75%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

Polynucleotides of the present invention may possess, when compared to naturally occurring molecules, one or more mutations which are deletions, insertions, or substitutions of nucleotide residues. Mutants can be either naturally occurring (that is to say, isolated from a natural source) or synthetic (for example, by performing site-directed mutagenesis on the nucleic acid).

Such polynucleotides may be prepared by any of a number of conventional techniques. The desired polynucleotide may be chemically synthesized using known techniques. DNA fragments also may be produced by restriction endonuclease digestion of a full length cloned DNA sequence, and isolated by electrophoresis on agarose gels. If necessary, oligonucleotides that reconstruct the 5′ or 3′ terminus to a desired point may be ligated to a DNA fragment generated by restriction enzyme digestion. Such oligonucleotides may additionally contain a restriction endonuclease cleavage site upstream of the desired coding sequence, and position an initiation codon (ATG) at the N-terminus of the coding sequence.

Polymerase chain reaction (PCR) procedure also may be employed to isolate and amplify a polynucleotide as described herein in any embodiment. Oligonucleotides that define the desired termini of the DNA fragment are employed as 5′ and 3′ primers. The oligonucleotides may additionally contain recognition sites for restriction endonucleases, to facilitate insertion of the amplified DNA fragment into an expression vector. PCR techniques are described in Saiki et al., Science 239:487 (1988); Recombinant DNA Methodology, Wu et al., eds., Academic Press, Inc., San Diego (1989), pp. 189-196; and PCR Protocols: A Guide to Methods and Applications, Innis et al., eds., Academic Press, Inc. (1990).

### Administration of HIF-1α Polypeptides and Polynucleotides

In a preferred embodiment of the invention, an HIF-1α polypeptide or active fragment thereof is administered with a biologically acceptable carrier.

The phrase, “biologically acceptable carrier” refers to any diluent, excipient, additive, or solvent which is either pharmaceutically accepted for use in the mammal for which a composition is formulated.

Routes of administration of the polypeptide or active fragment thereof include but are not limited to parenteral (for example, intravenous, intradermal, intraperitoneal or subcutaneous), oral, inhalational (for example, intranasal), transdermal (for example, topical), transmucosal, and rectal administration.

In a further preferred embodiment of the invention, the HIF-1α polynucleotide is inserted into a recombinant expression vector for the purposes of administration to the subject.

The term “recombinant expression vector” refers to a plasmid, virus or other vehicle known in the art that has been manipulated by insertion or incorporation of the HIF-1α genetic sequences. Such expression vectors contain a promoter which facilitates the efficient transcription in the host of the inserted genetic sequence. The expression vector typically contains an origin of replication, a promoter, as well as specific genes which allow phenotypic selection of the transformed cells.

As used herein, the term “promoter” is to be taken in its broadest context and includes the transcriptional regulatory sequences of a genomic gene, including the TATA box or initiator element, which is required for transcription initiation, with or without additional regulatory elements (i.e., upstream activating sequences, transcription factor binding sites, enhancers and silencers) which alter gene expression, e.g., in response to developmental and/or external stimuli, or in a tissue specific manner. In the present context, the term “promoter” is also used to describe a recombinant, synthetic or fusion molecule, or derivative which confers, activates or enhances the expression of a nucleic acid to which it is operably linked, and which encodes the peptide or protein. Preferred promoters can contain additional copies of one or more specific regulatory elements to further enhance expression and/or alter the spatial expression and/or temporal expression of said nucleic acid molecule.

Promoters useful with the subject invention include, for example, the cytomegalovirus immediate early promoter (CMV), the human elongation factor 1-α promoter (EF1), the small nuclear RNA promoters (U1a and U1b), α-myosin heavy chain promoter, Simian virus 40 promoter (SV40), Rous sarcoma virus promoter (RSV), Adenovirus major late promoter, β-actin promoter and hybrid regulatory element comprising a CMV enhancer/β-actin promoter. These promoters have been shown to be active in a wide range of mammalian cells.

Promoters particularly useful for expression of a protein in adipocytes or skeletal muscle cells or cells of the nervous system involved in regulation of energy intake and energy expenditure include, for example, the aP2 adipocyte specific promoter, MLC1F or MCK muscle specific promoters and the rab3, CaMKIIalpha, nestin or POMC nervous system specific promoters.

Also contemplated for use with the vectors of the present invention are inducible and cell type specific promoters. For example, Tet-inducible promoters (Clontech, Palo Alto, Calif.) and VP16-LexA promoters (Nettelbeck et al., 1998) can be used in the present invention.

The promoters are operably linked with heterologous DNA encoding HIF-1α. By “operably linked”, it is intended that the promoter element is positioned relative to the coding sequence to be capable of effecting expression of the coding sequence.

Preferred vectors can also include introns inserted into the polynucleotide sequence of the vector as a means for increasing expression of heterologous DNA encoding HIF-1α. For example, an intron can be inserted between a promoter sequence and the region coding for the protein of interest on the vector. Introns can also be inserted in the coding regions. Transcriptional enhancer elements which can function to increase levels of transcription from a given promoter can also be included in the vectors of the invention. Enhancers can generally be placed in either orientation, 3′ or 5′, with respect to promoter sequences. In addition to the natural enhancers, synthetic enhancers can be used in the present invention. For example, a synthetic enhancer randomly assembled from Spc5-12-derived elements including muscle-specific elements, serum response factor binding element (SRE), myocyte-specific enhancer factor-1 (MEF-1), myocyte-specific enhancer factor-2 (MEF-2), transcription enhancer factor-1 (TEF-1) and SP-1 (Li et al., 1999; Deshpande et al., 1997; Stewart et al., 1996; Mitchell and Tjian, 1989; Briggs et al., 1986; Pitluk et al., 1991) can be used in vectors of the invention.

Preferred viral vectors are derived from adeno-associated virus (AAV) and comprise a constitutive or regulatable promoter capable of driving sufficient levels of expression of the HIF-1 α-encoding DNA in the viral vector. Preferably, the viral vector comprises inverted terminal repeat sequences of AAV, such as those described in WO 93/24641. In a preferred embodiment, the viral vector comprises polynucleotide sequences of the pTR-UF5 plasmid. The pTR-UF5 plasmid is a modified version of the pTR_{BS}-UF/UF1/UF2/UFB series of plasmids (Zolotukiin et al., 1996; Klein et al., 1998). Nonlimiting examples of additional viral vectors useful according to this aspect of the invention include lentivirus vectors, herpes simplex virus vectors, adenovirus vectors, adeno-associated virus vectors, various suitable retroviral vectors, pseudorabies virus vectors, alpha-herpes virus vectors, HIV-derived vectors, other neurotropic viral vectors and the like.

Any means for the introduction of nucleic acids into a subject may be used in accordance with the methods described herein according to any embodiment.

Gene delivery vehicles useful in the practice of the present invention can be constructed utilizing methodologies known in the art of molecular biology, virology, microbiology, molecular biology and recombinant DNA techniques within the skill of the art. Preferred delivery systems are described below.

### a) Adeno-Associated Vectors

An exemplary viral vector system useful for delivery of a nucleic acid of the present invention is an adeno-associated virus (AAV). Human adenoviruses are double-stranded DNA viruses which enter cells by receptor-mediated endocytosis. These viruses have been considered well suited for gene transfer because they are easy to grow and manipulate and they exhibit a broad host range in vivo and in vitro. Adenoviruses are able to infect quiescent as well as replicating target cells and persist extrachromosomally, rather than integrating into the host genome. AAV is a helper-dependent DNA parvovirus which belongs to the genus Dependovirus. AAV has no known pathologies and is incapable of replication without additional helper functions provided by another virus, such as an adenovirus, vaccinia or a herpes virus, for efficient replication and a productive life cycle.

In the absence of the helper virus, AAV establishes a latent state by insertion of its genome into a host cell chromosome. Subsequent infection by a helper virus rescues the integrated copy which can then replicate to produce infectious viral progeny. The combination of the wild type AAV virus and the helper functions from either adenovirus or herpes virus generates a recombinant AVV (rAVV) that is capable of replication. One advantage of this system is its relative safety (For a review, see Xiao et al., (1997) Exp. Neurol. 144: 113-124).

Vectors containing as little as 300 base pairs of AAV can be packaged and can integrate. Space for exogenous DNA is limited to about 4.7 kb, which is sufficient to incorporate a nucleic acid encoding a polypeptide, fragment or analogue of the present invention. An AAV vector such as that described in Tratschin et al., (1985) Mol. Cell. Biol. 5: 3251-3260 can be used to introduce DNA into cells. A variety of nucleic acids have been introduced into different cell types using AAV vectors (see for example Hermonat et al., (1984) PNAS USA 81: 6466-6470; Tratschin et al., (1985) Mol. Cell. Biol. 4: 2072-2081; Wondisford et al., (1988) Mol. Endocrinol. 2: 32-39; Tratschin et al., (1984) J. Virol. 51: 611-619; and Flotte et al., (1993) J. Biol. Chem. 268: 3781-3790).

For additional detailed guidance on AAV technology which may be useful in the practice of the subject invention, including methods and materials for the incorporation of a nucleotide sequence, the propagation and purification of the recombinant AAV vector containing the nucleotide sequence, and its use in transfecting cells and mammals, see e.g. Carter et al, U.S. Pat. No. 4,797,368 (10 Jan. 1989); Muzyczka et al, U.S. Pat. No. 5,139,941 (18 Aug. 1992); Lebkowski et al, U.S. Pat. No. 5,173,414 (22 Dec. 1992); Srivastava, U.S. Pat. No. 5,252,479 (12 Oct. 1993); Lebkowski et al, U.S. Pat. No. 5,354,678 (11 Oct. 1994); Shenk et al, U.S. Pat. No. 5,436,146 (25 Jul. 1995); Chatterjee et al, U.S. Pat. No. 5,454,935 (12 Dec. 1995), Carter et al WO 93/24641 (published 9 Dec. 1993), and Natsoulis, U.S. Pat. No. 5,622,856 (Apr. 22, 1997).

### b) Adenoviral Vectors

In one example, a viral gene delivery system useful in the present invention utilizes adenovirus-derived vectors. Knowledge of the genetic organization of adenovirus, a 36 kB, linear and double-stranded DNA virus, allows substitution of a large piece of adenoviral DNA with foreign sequences up to 8 kB. The infection of adenoviral DNA into host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. Recombinant adenovirus is capable of transducing both dividing and non-dividing cells. The ability to effectively transduce non-dividing cells makes adenovirus a good candidate for gene transfer into muscle or fat cells.

The genome of an adenovirus can be manipulated such that it encodes a gene product of interest, but is inactivated in terms of its ability to replicate in a normal lytic viral life cycle (see, for example, Berkner et al., (1988) BioTechniques 6: 616; Rosenfeld et al., (1991) Science 252: 431-434; and Rosenfeld et al., (1992) Cell 68: 143-155). Suitable adenoviral vectors derived from the adenovirus strain Ad type 5 dl324 or other strains of adenovirus (e.g., Ad2, Ad3, Ad7 etc.) are known to those skilled in the art.

Recombinant adenoviruses can be advantageous in certain circumstances in that they are capable of infecting nondividing cells and can be used to infect a wide variety of cell types, including airway epithelium (Rosenfeld et al., (1992) cited supra), endothelial cells (Lemarchand et al., (1992) PNAS USA 89: 6482-6486), hepatocytes (Herz and Gerard, (1993) PNAS USA 90: 2812-2816) and muscle cells (Quantin et al., (1992) PNAS USA 89: 2581-2584; Ragot et al. (1993) Nature 361: 647).

Moreover, the carrying capacity of the adenoviral genome for foreign DNA is large (up to 8 kilobases) relative to other gene delivery vectors (Berkner et al., supra; Haj-Ahmand and Graham (1986) J. Virol. 57: 267). Most replication-defective adenoviral vectors currently in use and therefore favored by the present invention are deleted for all or parts of the viral E1 and E3 genes but retain as much as 80% of the adenoviral genetic material (see, e.g., Jones et al., (1979) Cell 16: 683; Berkner et al., supra; and Graham et al., in Methods in Molecular Biology, E. J. Murray, Ed. (Humana, Clifton, N.J., 1991) vol. 7. pp. 109-127). Expression of the inserted polynucleotide of the invention can be under control of, for example, the E1 A promoter, the major late promoter (MLP) and associated leader sequences, the viral E3 promoter, or exogenously added promoter sequences.

In certain embodiments, the adenovirus vector may be replication defective, or conditionally defective. The adenovirus may be of any of the 42 different known serotypes or subgroups A-F. Adenovirus type 5 of subgroup C is the exemplary starting material in order to obtain the conditional replication-defective adenovirus vector for use in accordance with the methods and compositions described herein. This is because Adenovirus type 5 is a human adenovirus about which a great deal of biochemical and genetic information is known, and it has historically been used for most constructions employing adenovirus as a vector. As stated above, the typical vector according to the present invention is replication defective and will not have an adenovirus E1 region. Thus, it will be most convenient to introduce the nucleic acid of interest at the position from which the E1 coding sequences have been removed. However, the position of insertion of the polynucleotide in a region within the adenovirus sequences is not critical to the present invention. For example, it may also be inserted in lieu of the deleted E3 region in E3 replacement vectors as described previously by Karlsson et. al. (1986) or in the E4 region where a helper cell line or helper virus complements the E4 defect.

An exemplary helper cell line is 293 (ATCC Accession No. CRL1573). This helper cell line, also termed a “packaging cell line” was developed by Frank Graham (Graham et al. (1987) J. Gen. Virol. 36: 59-72 and Graham (1977) J. General Virology 68: 937-940) and provides E1A and E1B in trans. However, helper cell lines may also be derived from human cells, such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells.

For additional detailed guidance on adenovirus technology which may be useful in the practice of the subject invention, including methods and materials for the incorporation of a nucleic acid, propagation and purification of recombinant virus containing the nucleic acid, and its use in transfecting cells and mammals, see also Wilson et al, WO 94/28938, WO 96/13597 and WO 96/26285, and references cited therein.

### c) Other Viral Systems

Other viral vector systems that can be used to deliver nucleic acid may be derived from, for example, a retrovirus (e.g., a lentivirus such as HIV), herpes virus, e. g., Herpes Simplex Virus (I J St U.S. Pat. No. 5,631,236 by Woo et al., issued May 20, 1997 and WO 00/08191 by Neurovex), vaccinia virus (Ridgeway (1988) Ridgeway, “Mammalian expression vectors,” In: Rodriguez R L, Denhardt D T, ed. Vectors: A survey of molecular cloning vectors and their uses. Stoneham: Butterworth; Baichwal and Sugden (1986)“Vectors for gene transfer derived from animal DNA viruses: Transient and stable expression of transferred genes,” In: Kucherlapati R, ed. Gene transfer. New York: Plenum Press; Coupar et al. (1988) Gene, 68: 1-10), and several RNA viruses. Exemplary viruses include, for example, an alphavirus, a poxivirus, a vaccinia virus, a polio virus, and the like. They offer several attractive features for various mammalian cells (Friedmann (1989) Science, 244: 1275-1281; Ridgeway, 1988, supra; Baichwal and Sugden, 1986, supra; Coupar et al., 1988; Horwich et al. (1990) J. Virol., 64: 642-650).

### d) Non-Viral Transfer.

Several non-viral methods for the transfer of nucleic acid into mammalian cells are also encompassed by the present invention. These include calcium phosphate precipitation (Graham and Van Der Eb, Virology, 52: 456-467,1973; Chen and Okayama, Mol. Cell. Biol., 7: 2745-2752, 1987; Rippe et al., Mol. Cell. Biol., 10: 689-695,1990) DEAE-dextran (Gopal, Mol. Cell. Biol., 5: 1188-1190,1985), electroporation (Tur-Kaspa et al., Mol. Cell. Biol., 6: 716-718, 1986; Potter et al., Proc. Natl. Acad. Sci. USA, 81: 7161-7165,1984), direct microinjection, DNA-loaded liposomes (Nicolau and Sene, Biochim. Biophys. Acta, 721: 185-190,1982; Fraley et al., Proc. Natl. Acad. Sci. USA, 76: 3348-3352,1979), cell sonication (Fechheimer et al., Proc. Natl. Acad. Sci. USA, 84: 8463-8467,1987), gene bombardment using high velocity microprojectiles (Yang et al., Proc. Natl. Acad. Sci. USA, 87: 9568-9572,1990), receptor-mediated transfection (Wu and Wu, J. Biol. Chem., 262: 4429-4432, 19877; Wu and Wu, Biochem., 27: 887-892,1988). In other embodiments, transfer of nucleic acids into cells may be accomplished by formulating the nucleic acids with nanocaps (e.g., nanoparticulate CaP04), colloidal gold, nanoparticulate synthetic polymers, and/or liposomes.

Once the construct has been delivered into the cell the nucleic acid of the invention may be positioned and expressed at different sites. In certain embodiments, the nucleic may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle.

In one example, a nucleic acid is entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, In: Liver diseases, targeted diagnosis and therapy using specific receptors and ligands, (Wu G, Wu C ed.), New York: Marcel Dekker, pp. 87-104, 1991). The addition of DNA to cationic liposomes causes a topological transition from liposomes to optically birefringent liquid-crystalline condensed globules (Radler et al., Science, 275: 810-814,1997). These DNA-lipid complexes are useful as non-viral vectors for use in gene therapy.

Liposome-mediated nucleic acid delivery and expression of foreign DNA in vitro has been very successful, and described, for example, in Wong et al. (Gene, 10: 87-94,1980).

In certain embodiments of the invention, the liposome may be complexed with a hemagglutinating virus (HVJ). This has been shown to facilitate fusion with the cell membrane and promote cell entry of liposome-encapsulated DNA (Kaneda et al., Science, 243: 375-378,1989). In other embodiments, the liposome may be complexed or employed in conjunction with nuclear nonhistone chromosomal proteins (HMG-1) (Kato et al, J. Biol. Chem., 266: 3361-3364,1991).

Other vector delivery systems which can be employed to deliver a nucleic acid encoding a therapeutic gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, Adv. Drug Delivery Rev., 12: 159-167,1993). Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, supra 1987) and transferrin (Wagner et al., Proc. Natl. Acad. Sci. USA 87 (9): 3410-3414,1990).

Another embodiment of the invention for transferring a nucleic acid into cells may involve particle bombardment. This method depends on the ability to accelerate DNA coated microprojectiles to a high velocity allowing them to pierce cell membranes and enter cells without killing them (Klein et al., Nature, 327: 70-73,1987). Several devices for accelerating small particles have been developed. One such device relies on a high voltage discharge to generate an electrical current, which in turn provides the motive force (Yang et al., supra 1990). The microprojectiles used have consisted of biologically inert substances such as tungsten or gold beads.

### e) Cells

In still another example, the present invention involves administering a cell expressing a polypeptide as described herein in any embodiment. Exemplary cell types include, for example, cells derived from a variety of tissues such as muscle, neural tissue or adipose tissue or a progenitor cell capable of differentiating into such a cell, e.g., myocytes (muscle stem cells), pluripotent stem cells, muscle derived stem cells, fat-derived stem cells, mesenchymal stem cells.

In exemplary embodiments, cells useful as bioactive agents are autologous to a subject to be treated. Alternatively, cells from close relatives or other donors of the same species may be used with appropriate immunosuppression. Immunologically inert cells, such as embryonic or fetal cells, stem cells, and cells genetically engineered to avoid the need for immunosuppression can also be used. Methods and drugs for immunosuppression are known to those skilled in the art of transplantation.

Suitable methods for modifying a cell to express a peptide or analogue of the invention are known in the art and/or described herein.

The dosage of recombinant vector or the virus or the cell to be administered to the subject can be determined by the ordinarily skilled clinician based on various parameters such as mode of administration, duration of treatment, the disease state or condition involved, and the like. Typically, recombinant virus of the invention is administered in doses between 10⁵ and 10¹⁴ infectious units. The recombinant vectors and virus of the present invention can be prepared in formulations using methods and materials known in the art. Numerous formulations can be found in Remington's Pharmaceutical Sciences, 15^{th} Edition (1975).

Inhibitors of Proteins that Mediate Degradation of HIF-1α

In one embodiment, the methods of the invention involve administering to the subject a partial or complete inhibitor of a protein that mediates degradation of HIF-1α.

In a preferred embodiment, the protein that mediates degradation of HIF-1α is a Von Hippel-Lindau protein (VHL). Preferably, the VHL protein has a sequence which shares at least 75% identity with a sequence as shown in any one of SEQ ID NO: 9 to 12.

In another preferred embodiment, the protein that mediates degradation of HIF-1α is a PHD 1 and/or PHD2 and/or PHD3. Preferably, the PHD 1 or PHD2 or PHD3 protein has a sequence which shares at least 75% identity with a sequence as shown in any one of SEQ ID NOs: 21 to 23.

In a preferred embodiment, the inhibitor of a protein that mediates degradation of HIF-1α is selected from the group consisting of an antisense polynucleotide, ribozyme, PNA, interfering RNA, siRNA, microRNA or antibody. These inhibitors are described in detail below. In a preferred embodiment, the inhibitor targets the portion of the VHL protein that binds to the oxygen degradation domain of HIF-1α.

### Antisense Polynucleotides

The term “antisense polynucleotide” shall be taken to mean a DNA or RNA, or combination thereof, that is complementary to at least a portion of a specific mRNA molecule encoding a polypeptide as described herein in any embodiment and capable of interfering with a post-transcriptional event such as mRNA translation. The use of antisense methods is known in the art (see for example, G. Hartmann and S. Endres, Manual of Antisense Methodology, Kluwer (1999)).

An antisense polynucleotide of the invention will hybridise to a target polynucleotide under physiological conditions. As used herein, the term “an antisense polynucleotide which hybridises under physiological conditions” means that the polynucleotide (which is fully or partially single stranded) is at least capable of forming a double stranded polynucleotide with mRNA encoding a protein, such as those encoding the VHL protein (the corresponding cDNA sequence of which is provided in any one of SEQ ID NO:13 to 16) or encoding a PHD protein (the corresponding cDNA sequence of which is provided in any one of SEQ ID NO:24 to 26) under normal conditions in a cell, preferably an adipocyte or a skeletal muscle cell or a cell of the nervous system involved in regulation of energy intake and energy expenditure.

Antisense molecules may include sequences that correspond to the structural genes or for sequences that effect control over the gene expression or splicing event. For example, the antisense sequence may correspond to the targeted coding region of the genes of the invention, or the 5′-untranslated region (UTR) or the 3′-UTR or combination of these. It may be complementary in part to intron sequences, which may be spliced out during or after transcription, preferably only to exon sequences of the target gene. In view of the generally greater divergence of the UTRs, targeting these regions provides greater specificity of gene inhibition.

The length of the antisense sequence should be at least 19 contiguous nucleotides, preferably at least 50 nucleotides, and more preferably at least 100, 200, 500 or 1000 nucleotides. The full-length sequence complementary to the entire gene transcript may be used. The length is most preferably 100-2000 nucleotides. The degree of identity of the antisense sequence to the targeted transcript should be at least 90% and more preferably 95-100%. The antisense RNA molecule may of course comprise unrelated sequences which may function to stabilise the molecule.

### Catalytic Polynucleotides

The term “catalytic polynucleotide/nucleic acid” refers to a DNA molecule or DNA-containing molecule (also known in the art as a “deoxyribozyme”) or an RNA or RNA-containing molecule (also known as a “ribozyme”) which specifically recognises a distinct substrate and catalyses the chemical modification of this substrate. The nucleic acid bases in the catalytic nucleic acid can be bases A, C, G, T (and U for RNA).

Typically, the catalytic nucleic acid contains an antisense sequence for specific recognition of a target nucleic acid, and a nucleic acid cleaving enzymatic activity (also referred to herein as the “catalytic domain”). The types of ribozymes that are particularly useful in this invention are a hammerhead ribozyme (Haseloff and Gerlach, 1988; Perriman et al., 1992) and a hairpin ribozyme (Zolotukiin et al., 1996; Klein et al., 1998; Shippy et al., 1999).

The ribozymes of this invention and DNA encoding the ribozymes can be chemically synthesised using methods well known in the art. The ribozymes can also be prepared from a DNA molecule (that upon transcription, yields an RNA molecule) operably linked to an RNA polymerase promoter, for example, the promoter for T7 RNA polymerase or SP6 RNA polymerase. Accordingly, also provided by this invention is a nucleic acid molecule, that is, DNA or cDNA, coding for a catalytic polynucleotide of the invention. When the vector also contains an RNA polymerase promoter operably linked to the DNA molecule, the ribozyme can be produced in vitro upon incubation with RNA polymerase and nucleotides. In a separate embodiment, the DNA can be inserted into an expression cassette or transcription cassette. After synthesis, the RNA molecule can be modified by ligation to a DNA molecule having the ability to stabilise the ribozyme and make it resistant to RNase.

As with antisense polynucleotides described herein, catalytic polynucleotides of the invention should also be capable of “hybridising” a target nucleic acid molecule (for example an mRNA encoding a VHL polypeptide (the corresponding cDNA sequences of which is provided in any one of SEQ ID NO: 13 to 16) or encoding a PHD protein (the corresponding cDNA sequence of which is provided in any one of SEQ ID NO:24 to 26) under “physiological conditions”, namely those conditions within a cell (especially conditions in an adipocyte or a skeletal muscle cell or a cell of the nervous system involved in regulation of energy intake and energy expenditure).

### RNA Interference

RNA interference (RNAi) is particularly useful for specifically inhibiting the production of a particular protein. Although not wishing to be limited by theory, Waterhouse et al. (1998) have provided a model for the mechanism by which dsRNA (duplex RNA) can be used to reduce protein production. This technology relies on the presence of dsRNA molecules that contain a sequence that is essentially identical to the mRNA of the gene of interest or part thereof, in this case an mRNA encoding a protein that mediates degradation of HIF-1α. Conveniently, the dsRNA can be produced from a single promoter in a recombinant vector or host cell, where the sense and anti-sense sequences are flanked by an unrelated sequence which enables the sense and anti-sense sequences to hybridise to form the dsRNA molecule with the unrelated sequence forming a loop structure. The design and production of suitable dsRNA molecules for the present invention is well within the capacity of a person skilled in the art, particularly considering Waterhouse et al. (1998), Smith et al. (2000), WO 99/32619, WO 99/53050, WO 99/49029, and WO 01/34815.

In one example, a DNA is introduced that directs the synthesis of an at least partly double stranded RNA product(s) with homology to the target gene to be inactivated. The DNA therefore comprises both sense and antisense sequences that, when transcribed into RNA, can hybridise to form the double stranded RNA region. In a preferred embodiment, the sense and antisense sequences are separated by a spacer region that comprises an intron which, when transcribed into RNA, is spliced out. This arrangement has been shown to result in a higher efficiency of gene silencing. The double stranded region may comprise one or two RNA molecules, transcribed from either one DNA region or two. The presence of the double stranded molecule is thought to trigger a response from an endogenous mammalian system that destroys both the double stranded RNA and also the homologous RNA transcript from the target mammalian gene, efficiently reducing or eliminating the activity of the target gene.

The length of the sense and antisense sequences that hybridise should each be at least 19 contiguous nucleotides, preferably at least 30 or 50 nucleotides, and more preferably at least 100, 200, 500 or 1000 nucleotides. The full-length sequence corresponding to the entire gene transcript may be used. The lengths are most preferably 100-2000 nucleotides. The degree of identity of the sense and antisense sequences to the targeted transcript should be at least 85%, preferably at least 90% and more preferably 95-100%. The RNA molecule may of course comprise unrelated sequences which may function to stabilise the molecule. The RNA molecule may be expressed under the control of a RNA polymerase II or RNA polymerase III promoter. Examples of the latter include tRNA or snRNA promoters.

Preferred small interfering RNA (“siRNA”) molecules comprise a nucleotide sequence that is identical to about 19-21 contiguous nucleotides of the target mRNA. Preferably, the siRNA sequence commences with the dinucleotide AA, comprises a GC-content of about 30-70% (preferably, 30-60%, more preferably 40-60% and more preferably about 45%-55%), and does not have a high percentage identity to any nucleotide sequence other than the target in the genome of the mammal in which it is to be introduced, for example as determined by standard BLAST search. Examples of siRNA molecules that target VHL mRNA are provided in any one of SEQ ID NO:17 to 20.

### MicroRNA

MicroRNA regulation is a clearly specialised branch of the RNA silencing pathway that evolved towards gene regulation, diverging from conventional RNAi/PTGS. MicroRNAs are a specific class of small RNAs that are encoded in gene-like elements organised in a characteristic inverted repeat. When transcribed, microRNA genes give rise to stem-looped precursor RNAs from which the microRNAs are subsequently processed. MicroRNAs are typically about 21 nucleotides in length. The released miRNAs are incorporated into RISC-like complexes containing a particular subset of Argonaute proteins that exert sequence-specific gene repression (see, for example, Millar and Waterhouse, 2005; Pasquinelli et al., 2005; Almeida and Allshire, 2005).

### Polyclonal and Monoclonal Antibodies

The term “antibody” as used herein includes intact molecules as well as fragments thereof, such as Fab and F(ab′)2, Fv and single chain antibody fragments capable of binding an epitopic determinant of an immunogen, e.g., pVHL or a PHD protein. This term also encompasses recombinant antibodies, chimeric antibodies and humanized antibodies.

An “Fab fragment” consists of a monovalent antigen-binding fragment of an antibody molecule, and can be produced by digestion of a whole antibody molecule with the enzyme papain, to yield a fragment consisting of an intact light chain and a portion of a heavy chain. An “Fab′ fragment” of an antibody molecule can be obtained by treating a whole antibody molecule with pepsin, followed by reduction, to yield a molecule consisting of an intact light chain and a portion of a heavy chain. Two Fab′ fragments are obtained per antibody molecule treated in this manner. An “F(ab′)2 fragment” of an antibody consists of a dimer of two Fab′ fragments held together by two disulfide bonds, and is obtained by treating a whole antibody molecule with the enzyme pepsin, without subsequent reduction. A (Fab′)₂ fragment. An “Fv fragment” is a genetically engineered fragment containing the variable region of a light chain and the variable region of a heavy chain expressed as two chains. A “single chain antibody” (SCA) is a genetically engineered single chain molecule containing the variable region of a light chain and the variable region of a heavy chain, linked by a suitable, flexible polypeptide linker.

If polyclonal antibodies are desired, a selected mammal (for example, mouse, rabbit, goat, horse, etc.) is immunised with an immunogenic polypeptide such as VHL (for example, as shown in any one of SEQ ID NO:9 to 12) or a PHD protein (for example, as set forth in any one of SEQ ID Nos: 21 to 23). Serum from the immunised animal is collected and treated according to known procedures. If serum containing polyclonal antibodies contains antibodies to other antigens, the polyclonal antibodies can be purified by immunoaffinity chromatography. Techniques for producing and processing polyclonal antisera are known in the art. In order that such antibodies may be made, the invention also provides peptides of the invention or fragments thereof haptenised to another peptide for use as immunogens in animals.

Monoclonal antibodies directed against a protein that mediates the degradation of HIF-1 α can also be readily produced by one skilled in the art. The general methodology for making monoclonal antibodies by hybridomas is known and described, for example, in Kohler and Milstein Nature 256:495-497, 1975; Brown et al. J. Immunol. 127:53946, 1981; Brown et al. J. Biol. Chem. 255: 4980-4983, 1980; Yeh et al. Proc. Natl. Acad. Sci. USA 76:2927-2931, 1976; Yeh et al. Int. J. Cancer 29: 269-275, 1982; Kozbor et al. Immunol Today 4:72, 1983; Cole et al., Monoclonal Antibodies and Cancer Therapy, Alan R. Liss, Inc., pp. 77-96, 1985. Briefly, an immortal cell line (typically a myeloma) is fused to lymphocytes (typically splenocytes) from a mammal immunized with a immunogen as described herein, and the culture supernatants of the resulting hybridoma cells are screened to identify a hybridoma producing a monoclonal antibody that binds the immunogen. Any of the known protocols used for fusing lymphocytes and immortalized cell lines can be applied for the purpose of generating a monoclonal antibody (see, e.g., G. Galfre et al., Nature 266: 550-552, 1970). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O—Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind the immunogen, e.g., using a standard ELISA assay. The antibodies can then be tested for suitability in a method as described herein according to any embodiment

An alternative technique involves screening phage display libraries where, for example the phage express scFv fragments on the surface of their coat with a large variety of complementarity determining regions (CDRs). This technique is known in the art.

### Chelating Agents

In one preferred embodiment of the invention, the level or stability of HIF-1α activity is increased by administering to the subject a chelating agent.

In a preferred embodiment, the chelating agent is an iron chelating agent or iron chelator (these terms are used interchangeably and one term will provide support for the other term in the context of this specification and the accompanying claims).

Iron chelators are known in the art and will be apparent to the skilled artisan and/or described herein. According to the observed binding to iron, the iron chelators may be classified into bidentate, tridentate or higher order multidentate chelators.

Exemplary bidentate iron chelators include 1,2-dimethyl-3-hydroxypyridin-4-one (Deferiprone, DFP or Ferriprox) or 2-deoxy-2-(N-carbamoylmethyl-[N′-2′-methyl-3′-hydroxypyridin-4′-one])-D-glucopyranose (Feralex-G).

Exemplary tridentate iron chelators comprise pyridoxal isonicotinyl hydrazone (P1H), 4,5-dihydro-2-(2,4-dihydroxyphenyl)-4-methylthiazole-4-carboxylic acid (GT56-252), 4,5-dihydro-2-(3′-hydroxypyridin-2′-yl)-4-methylthiazole-4-carboxylic acid (desferrithiocin or DFT) and 4-[3,5-bis(2-hydroxyphenyl)-[1,2,4]triazol-1-yl]benzoic acid (ICL-670). Substituted 3,5-diphenyl-1,2,4-triazoles in the free acid form, salts thereof and its crystalline forms are disclosed in the International Patent Publication WO 97/49395, which is hereby incorporated by reference. Similarly a particularly advantageous pharmaceutical preparation of such compounds in the form of dispersible tablets is disclosed in the International Patent Publication WO 2004/035026, which is also hereby incorporated by reference.

Exemplary hexadentate iron chelators comprise N,N′-bis(o-hydroxybenzyl)ethylenediamine-N,N′-diacetic acid (HBED), N-(5-C3-L (5 aminopentyl)hydroxycarbamoyl)-propionamido)pentyl)-3(5-(N-hydroxyacetoamido)-pentyl)carbamoyl)-proprionhydroxamic acid (deferoxamine, desferrioxamine or DFO) and hydroxymethyl-starch-bound deferoxamine (S-DFO). Further derivatives of DFO include aliphatic, aromatic, succinic, and methylsulphonic analogs of DFO and specifically, sulfonamide-deferoxamine, acetamide-deferoxamine, propylamide deferoxamine, butylamide-deferoxamine, benzoylamide-deferoxamine, succinamide-derferoxamine, and methylsulfonamide-deferoxamine.

A further class of iron chelators is the biomimetic class (Meijler, M M, et al. “Synthesis and Evaluation of Iron Chelators with Masked Hydrophilic Moieties” J. Amer. Chem. Soc. 124:1266-1267 (2002), is hereby incorporated by reference in its entirety). These molecules are modified analogues of such naturally produced chelators as DFO and ferrichrome. The analogues allow attachment of lipophilic moieties (e.g., acetoxymethyl ester). The lipophilic moieties are then cleaved intracellularly by endogenous esterases, converting the chelators back into hydrophilic molecules which cannot leak out of the cell.

Another class of iron chelators is the non-naturally-occurring iron chelators, such as siderophores and xenosiderophores. Siderophores and xenosiderophores include, for example, hydroxamates and polycarboxylates. The hydroxamates contain an N-δ-hydroxyornithine moiety and are generally categorized into four exemplary families. One category includes rhodotoruic acid, which is the diketopiperazine of N-δ-acetyl-L-N δ-hydroxyornithine. Included within this category are derivatives such as dihydroxamate named dimerum acid. A second category includes the coprogens, which contain an N-δ-acyl-N-δ-hydroxy-L-ornithine moiety.

Coprogens also can be considered trihydroxamate derivatives of rhodotorulic acid with a linear structure. A third category includes the ferrichromes, which consist of cyclic peptides containing a tripeptide of N-δ-acyl-N-δ-hydroxyornithine and combinations of glycine, serine or alanine. The fourth exemplary category includes the fusarinines, also called fusigens, which can be either linear or cyclic hydroxamates. Fusarinine is a compound characterized by N acylation of N-hydroxyornithine by anhydromevalonic acid.

The polycarboxylates consist of a citric acid-containing polycarboxylate called rhizoferrin. The molecule contains two citric acid units linked to diaminobutane. Rhizoferrin is widely distributed among the members of the phylum Zygomycota, having been observed in the order Mucorales and in the order Entomophthoraies. Other categories of siderophores useful as iron chelating compounds in the compositions of the invention include, for example, the phenolate-catecholate class of siderophores, hemin, and β-ketoaldehyde phytotoxins.

The iron chelator is preferably selected from the group consisting of deferasirox (DFS), desferrioxamine (DFO), ferrioxamine, trihydroxamic acid, CP94, EDTA, desferrioxamine hydroxamic acids, deferoxamine B (DFO) as the methanesulfonate salt, also known as desferrioxamine B mesylate (DFOM), desferal from Novartis (previously Ciba-Giegy), apoferritin, CDTA (trans-1,2-diaminocyclohexane-N,N,N,N′-tetraacetic acid), and DTPA (diethylenetriamine-N,N,N′,N″,N″-penta-acetic acid) and cobaltous ions.

Additional iron chelators are described, for example, in U.S. Pat. No. 5,047,421 (1991); U.S. Pat. No. 5,424,057 (1995); U.S. Pat. No. 5,721,209 (1998); U.S. Pat. No. 5,811,127 (1998); Olivieri, N. F. et al, New Eng. J. Med. 332:918-922 (1995); Boyce, N. W. et al, Kidney International. 50:813-817 (1986); Kontoghiorghes, G J. Indian J. Peditr. 60:485-507 (1993); Hershko, C. et al Brit. J. Haematology 101:399-406 (1998); Lowther, N. et al., Pharmac. Res. 16:434 (1999); Cohen, A. R., et al., Am. Soc. Hematology pages 14-34 (2004)); U.S. Pat. No. 6,993,104 (2005); U.S. Pat. No. 6,908,733 (2005); U.S. Pat. No. 6,906,052 (2005), the teachings of all of which are hereby incorporated by reference in their entirety.

In a preferred embodiment, the iron chelator is a substituted 3,5-diphenyl-1,2,4-triazole in the free acid form, a salt thereof or its crystalline form as disclosed in the International Patent Publications WO 97/49395 and WO2008/008537, which is hereby incorporated by reference. For example, the iron chelator is a compound of formula (I):

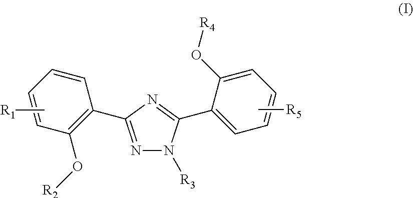

in which

R₁ and R₅ simultaneously or independently of one another are hydrogen, halogen, hydroxyl, lower alkyl, halo-lower alkyl, lower alkoxy, halo-lower alkoxy, carboxyl, carbamoyl, N-lower alkylcarbamoyl, N,N-di-lower alkylcarbamoyl or nitrile; R₂ and R₄ simultaneously or independently of one another are hydrogen, unsubstituted or substituted lower alkanoyl or aroyl, or a radical which can be removed under physiological conditions; R₃ is hydrogen, lower alkyl, hydroxy-Iower alkyl, halo-lower alkyl, carboxy-lower alkyl, lower alkoxycarbonyl-lower alkyl, R₆R₇N—C(O)-lower alkyl, unsubstituted or substituted aryl or aryl-lower alkyl, or unsubstituted or substituted heteroaryl or heteroaralkyl; R₆ and R₇ simultaneously or independently of one another are hydrogen, lower alkyl, hydroxy-lower alkyl, alkoxy-lower alkyl, hydroxyalkoxy-lower alkyl, amino-lower alkyl, N-lower alkylamino-lower alkyl, N,N-di-lower alkylamino-lower alkyl, N-(hydroxy-lower alkyl)amino-lower alkyl, N,N-di(hydroxy-lower alkyl)amino-lower alkyl or, together with the nitrogen atom to which they are bonded, form an azaalicyclic ring; or a pharmaceutically acceptable salt thereof.

Halogen is, for example, chlorine, bromine or fluorine, but can also be iodine.

The prefix “lower” designates a radical having not more than 7 and in particular not more than 4 carbon atoms.

Alkyl is straight-chain or branched. Per se, for example lower alkyl, or as a constituent of other groups, for example lower alkoxy, lower alkylamine, lower alkanoyl, lower alkylaminocarbonyl, it can be unsubstituted or substituted, for example by halogen, hydroxyl, lower alkoxy, trifluoromethyl, cyclo-lower alkyl, azaalicyclyl or phenyl, it is preferably unsubstituted or substituted by hydroxyl.

Lower alkyl is, for example, n-propyl, isopropyl, n-butyl, isobutyl, sec-butyl, tert-butyl, n-pentyl, neopentyl, n-hexyl or n-heptyl, preferably methyl, ethyl and n-propyl. Halo-lower alkyl is lower alkyl substituted by halogen, preferably chlorine or fluorine, in particular by up to three chlorine or fluorine atoms.

Lower alkoxy is, for example, n-propoxy, isopropoxy, n-butoxy, isobutoxy, sec-butoxy, tert-butoxy, n-amyloxy, isoamyloxy, preferably methoxy and ethoxy. Halo-lower alkoxy is lower alkoxy substituted by halogen, preferably chlorine or fluorine, in particular by up to three chlorine or fluorine atoms.

Carbamoyl is the radical HaN-C(O)—, N-lower alkylcarbamoyl is lower alkyl-HN—C(O)— and N,N-di-lower alkylcarbamoyl is di-lower alkyl-N—C(O)—.

Lower alkanoyl is HC(O)— and lower alkyl-C(O)— and is, for example, acetyl, propanoyl, butanoyl or pivaloyl.

Lower alkoxycarbonyl designates the radical lower alkyl-O—C(O)— and is, for example, M-propoxycarbonyl, isopropoxycarbonyl, n-butoxycarbonyl, isobutoxycarbonyl, sec-butoxycarbonyl, tert-butoxycarbonyl, n-amyloxycarbonyl, isoamyloxycarbonyl, preferably methoxycarbonyl and ethoxycarbonyl.

Aryl, per se, for example aryl, or as a constituent of other groups, for example aryl-lower alkyl or aroyl, is, for example, phenyl or naphthyl, which is substituted or unsubstituted. Aryl is preferably phenyl which is unsubstituted or substituted by one or more, in particular one or two, substituents, for example lower alkyl, lower alkoxy, hydroxyl, nitro, amino, halogen, trifluoromethyl, carboxyl, lower alkoxycarbonyl, amino, N-lower alkylamino, N,N-di-lower alkylamino, aminocarbonyl, lower alkylaminocarbonyl, di-lower alkylaminocarbonyl, heterocycloalkyl, heteroaryl or cyano. Primarily, aryl is unsubstituted phenyl or naphthyl, or phenyl which is substituted by lower alkyl, lower alkoxy, hydroxyl, halogen, carboxyl, lower alkoxycarbonyl, N,N-di-lower alkylamino or heterocycloalkylcarbonyl.

Aroyl is the radical aryl-C(O)— and is, for example, benzoyl, toluoyl, naphthoyl or p-methoxy benzoyl.

Aryl-lower alkyl is, for example, benzyl, p-chlorobenzyl, o-fluorobenzyl, phenylethyl, p-tolylmethyl, p-dimethylaminobenzyl, p-diethylaminobenzyl, p-cyanobenzyl, p-pyrrolidinobenzyl.

Heterocycloalkyl designates a cycloalkyl radical having 3 to 8, in particular having from 5 to not more than 7, ring atoms, of which at least one is a heteroatom; oxygen, nitrogen and sulfur are preferred. Azaalicyclyl is a saturated cycloalkyl radical having 3-8, in particular 5-7, ring atoms, in which at least one of the ring atoms is a nitrogen atom. Azaalicyclyl can also contain further ring heteroatoms, e.g. oxygen, nitrogen or sulfur; it is, for example, piperidinyl, piperazinyl, morpholinyl or pyrrolidinyl. Azaalicyclyl radicals can be unsubstituted or substituted by halogen or lower alkyl. The azaalicyclyl radicals bonded via a ring nitrogen atom, which are preferred, are, as is known, designated as piperidino, piperazino, morpholino, pyrrolidino etc.

Heteroaryl per se, for example heteroaryl, or as a constituent of other substituents, for example heteroaryl-lower alkyl, is an aromatic radical having from 3 to not more than 7, in particular from 5 to not more than 7, ring atoms, in which at least one of the ring atoms is a heteroatom, e.g. pyrrolyl, imidazolyl, triazolyl, tetrazolyl, oxazolyl, thiazolyl, furanyl, thiophenyl, pyridyl, pyrazinyl, oxazinyl, thiazinyl, pyranyl or pyrimidinyl. Heteroaryl can be substituted or unsubstituted. Heteroaryl which is unsubstituted or substituted by one or more, in particular one or two, substituents, for example lower alkyl, halogen, trifluoromethyl, carboxyl or lower alkoxycarbonyl, is preferred.

Heteroaryl-lower alkyl designates a lower alkyl radical in which at least one of the hydrogen atoms, preferably on the terminal C atom, is replaced by a heteroaryl group if the alkyl chain contains two or more carbon atoms.

N-lower alkylamino is, for example, n-propylamino, n-butylamino, propylamino, i-butyl-amino, hydroxyethylamino, preferably methylamino and ethylamino. In N,N-di-lower alkylamino, the alkyl substituents can be identical or different. Thus N,N-di-lower alkylamino is, for example, N,N-dimethylamino, N,N-diethylamino, N,N-methylethylamino, N-methyl-N-morpholinoethylamino, N-methyl-N-hydroxyethylamino, N-methyl-N-benzylamino.

Salts of compounds of the formula (I) are pharmaceutically acceptable salts, especially salts with bases, such as appropriate alkali metal or alkaline earth metal salts, e.g. sodium, potassium or magnesium salts, pharmaceutically acceptable transition metal salts such as zinc salts, or salts with organic amines, such as cyclic amines, such as mono-, di- or tri-lower alkylamines, such as hydroxy-lower alkylamines, e.g. mono-, di- or trihydroxy-lower alkylamines, hydroxy-lower alkyl-lower alkylamines or polyhydroxy-lower alkylamines. Cyclic amines are, for example, morpholine, thiomorpholine, piperidine or pyrrolidine. Suitable mono-lower alkylamines are, for example, ethyl- and ferf-butylamine; di-lower alkylamines are, for example, diethyl- and diisopropylamine; and tri-lower alkylamines are, for example, trimethyl- and triethylamine. Appropriate hydroxy-lower alkylamines are, for example, mono-, di- and triethanolamine; hydroxy-lower alkyl-lower alkylamines are, for example, N,N-dimethylamino- and N,N-diethylaminoethanol; a suitable polyhydroxy-lower alkylamine is, for example, glucosamine. In other cases it is also possible to form acid addition salts, for example with strong inorganic acids, such as mineral acids, e.g. sulfuric acid, a phosphoric acid or a hydrohalic acid, with strong organic carboxylic acids, such as lower alkanecarboxylic acids, e.g. acetic acid, such as saturated or unsaturated dicarboxylic acids, e.g. malonic, maleic or fumaric acid, or such as hydroxycarboxylic acids, e.g. tartaric or citric acid, or with sulfonic acids, such as lower alkane- or substituted or unsubstituted benzenesulfonic acids, e.g. methane- or p-toluenesulfonic acid. Compounds of the formula (I) having an acidic group, e.g. carboxyl, and a basic group, e.g. amino, can also be present in the form of internal salts, i.e. in zwitterionic form, or a part of the molecule can be present as an internal salt, and another part as a normal salt.

Preferably, the iron chelator is a compound of formula (I), in which R₁ and R₅ simultaneously or independently of one another are hydrogen, halogen, hydroxyl, lower alkyl, halo-lower alkyl, lower alkoxy or halo-lower alkoxy; R₂ and R₄ simultaneously or independently of one another are hydrogen or a radical which can be removed under physiological conditions; R₃ is lower alkyl, hydroxy-lower alkyl, carboxy-lower alkyl, lower alkoxycarbonyl-lower alkyl, R₆R₇N—CO-lower alkyl, substituted aryl, aryl-lower alkyl, substituted by N-lower alkylamino, N,N-di-lower alkylamino or pyrrolidino, or unsubstituted or substituted heteroaryl or heteroaralkyl; R₆ and R₇ simultaneously or independently of one another are hydrogen, lower alkyl, hydroxy-lower alkyl, alkoxy-lower alkyl, hydroxyalkoxy-lower alkyl, amino-lower alkyl, N-lower alkylamino-lower alkyl, N,N-di-lower alkylamino-lower alkyl, N-(hydroxy-lower alkyl)amino-lower alkyl, N,N-di (hydroxy-lower alkylamino-lower alkyl or, together with the nitrogen atom to which they are bonded, form an azaalicyclic ring; or a salt thereof.

In one embodiment of the invention, the compound of formula (I) is 4-[3,5-bis(2-hydroxyphenyl)-[1,2,4]triazol-1-yl]benzoic acid (e.f., as depicted in Formula (II)) or a pharmaceutically acceptable salt.

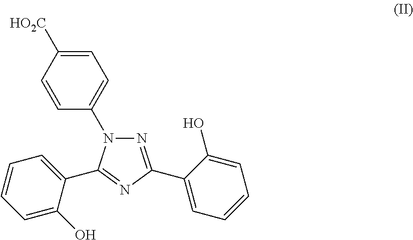

### Pharmaceutical Formulations

Pharmaceutical preparations for enteral or parenteral administration are, for example, those in unit dose forms, such as sugar-coated tablets, tablets, dispersible tablets, effervescent tablets, capsules, suspendable powders, suspensions or suppositories, or ampoules. These are prepared in a manner known per se, e.g. by means of conventional pan-coating, mixing, granulation or lyophilization processes. Pharmaceutical preparations for oral administration can thus be obtained by combining the active ingredient with solid carriers, if desired granulating a mixture obtained and processing the mixture or granules, if desired or necessary, after addition of suitable adjuncts to give tablets or sugar-coated tablet cores.

Suitable carriers are, in particular, fillers such as sugars, e.g. lactose, sucrose, mannitol or sorbitol, cellulose preparations and/or calcium phosphates, e.g. tricalcium phosphate or calcium hydrogen phosphate, furthermore binders, such as starch pastes, using, for example, maize, wheat, rice or potato starch, gelatin tragacanth, methylcellulose and/or polyvmylpyrroiidone, and, if desired, disintegrants, such as the abovementioned starches, furthermore carboxymethyl starch, crosslinked polyvmylpyrroiidone, agar or alginic acid or a salt thereof, such as sodium alginate. Adjuncts are primarily flow-regulating and lubricating agents, e.g. salicylic acid, talc, stearic acid or salts thereof, such as magnesium or calcium stearate, and/or polyethylene glycol. Sugar-coated tablet cores are provided with suitable, if desired enteric, coatings, using, inter alia, concentrated sugar solutions which, if desired, contain gum arable, talc, polyvmylpyrroiidone, polyethylene glycol and/or titanium dioxide, coating solutions in suitable organic solvents or solvent mixtures or, for the preparation of enteric coatings, solutions of suitable cellulose preparations, such as acetylcellulose phthalate or hydroxypropylmethylcellulose phthalate. Colorants or pigments, e.g. for the identification or the marking of various doses of active ingredient, can be added to the tablets or sugar-coated tablet coatings.

Dispersible tablets are tablets which rapidly disintegrate in a comparatively small amount of liquid, e.g. water, and which, if desired, contain flavourings or substances for masking the taste of the active ingredient. They can advantageously be employed for the oral administration of large individual doses, in which the amount of active ingredient to be administered is so large that on administration as a tablet which is to be swallowed in undivided form or without chewing that it can no longer be conveniently ingested, in particular by children. Further orally administrable pharmaceutical preparations are hard gelatin capsules and also soft, closed capsules of gelatin and a plasticizer, such as glycerol or sorbitol The hard gelatin capsules can contain the active ingredient in the form of granules, e.g. as a mixture with fillers, such as lactose, binders, such as starches, and/or glidants, such as talc or magnesium stearate, and if desired, stabilizers. In soft capsules, the active ingredient is preferably dissolved or suspended in suitable liquids, such as fatty oils, liquid paraffin or liquid polyethylene glycols, it also being possible to add stabilizers.

Preferred dispersable tablets are described in, for example, International Patent Publication No. WO2008/015021. By “dispersible tablet” is meant a tablet which disperses in aqueous phase, e.g. in water, before administration. For example, the dispersible tablet has high drug loading, e.g., comprising a compound of Formula I or II I as active ingredient, the active ingredient being present in an amount of from about 5% to 40%, e.g. at least about 10, 15, 20 or 25%, preferably more than 25% in weight based on the total weight of the dispersible tablet. In particular, the amount of active ingredient may vary from 25 to 40%, e.g. 28 to 32% in weight based on the total weight of the dispersible tablet. The active ingredient may be in the free acid form or pharmaceutically acceptable salts thereof, preferably in the free acid form. One or more pharmaceutically acceptable excipients may be present in the dispersible tablets, e.g. those conventionally used, e.g. at least one filler, e.g., lactose, ethylcellulose, microcrystalline cellulose, at least one disintegrant, e.g. cross-linked polyvinylpyrrolidinone, e.g. Crospovidone, at least one binder, e.g. polyvinylpyridone, hydroxypropylmethyl cellulose, at least one surfactant, e.g. sodium laurylsulfate, at least one glidant, e.g. colloidal silicon dioxide, at least one lubricant, e.g. magnesium stearate.

The chelating agent may also be provided as suspendable powders, e.g., those which are described as “powder in bottle”, abbreviated “PIB”, or ready-to-drink suspensions, are suitable for an oral administration form. For this form, the active ingredient is mixed for example, with pharmaceutically acceptable surface-active substances, for example sodium lauryl sulfate or polysorbate, suspending auxiliaries, e.g. hydroxypropylcellulose, hydroxypropylmethylcellulose or another known from the prior art and previously described, for example, in “Handbook of Pharmaceutical Ecipients”, pH regulators, such as citric or tartanc acid and their salts or a USP buffer and, if desired, fillers, e.g. lactose, and further auxiliaries, and dispensed into suitable vessels, advantageously single-dose bottles or ampoules. Immediately before use, a specific amount of water is added and the suspension is prepared by shaking. Alternatively, the water can also be added even before dispensing

Rectally administrable pharmaceutical preparations are, for example, suppositories which consist of a combination of the active ingredient with a suppository base. A suitable suppository base is, for example, natural or synthetic triglycerides, paraffin hydrocarbons, polyethylene glycols or higher alkanols. Gelatin rectal capsules can also be used which contain a combination of the active ingredient with a base substance. Possible base substances are, for example, liquid triglycerides, polyethylene glycols or paraffin hydrocarbons.

For parenteral administration, aqueous solutions of an active ingredient in water-soluble form, e.g. of a water-soluble salt, are primarily suitable; furthermore suspensions of the active ingredient, such as appropriate oily injection suspensions, suitable lipophilic solvents or vehicles, such as fatty oils, e.g. sesame oil, or synthetic fatty acid esters, e.g. ethyl oleate or triglycerides, being used, or aqueous injection suspensions which contain viscosity-increasing substances, e.g. sodium carboxymethylcellulose, sorbitol and/or dextran, and, if desired, also stabilizers.

The chelating agent may be administered by any suitable route. Routes of administration of the chelating agent include intramuscular, parenteral (including intravenous), intra-arterial, subcutaneous, oral, and nasal administration.

### Dosage and Mode of Administration

The dosage of the active ingredient can depend on various factors, such as activity and duration of action of the active ingredient, severity of the illness to be treated or its symptoms, manner of administration, species, sex, age, weight and/or individual condition of the subject to be treated. Preferably, the chelating agent is administered in at least one dose that is within the range 0.0001 to 1.0 mg/kg. In the case of a compound of formula I or II the doses to be administered daily in the case of oral administration are between 10 and approximately 120 mg/kg, in particular 20 and approximately 80 mg/kg, and for a warm-blooded animal having a body weight of approximately 40 kg, preferably between approximately 400 mg and approximately 4,800 mg, in particular approximately 800 mg to 3.200 mg, which is expediently divided into 2 to 12 individual doses.

In a preferred embodiment, the iron chelator is desferrioxamine (DFO) or a derivative thereof, e.g., as described in International Patent Publication No. WO1985/003290.

In a further preferred embodiment the DFO is administered intravenously, diluted in normal saline. Preferably, the dose is within the range 5g to 10 g per person. Preferably the dose is administered once weekly.

In yet another embodiment the DFO is administered by subcutaneous infusion.

In another embodiment, the iron chelator is administered orally, for example, in the form of the once-daily oral iron chelator Exjade (deferasirox (DFS)).

Short-term or long-term administration of chelating agents is contemplated by the present invention, depending upon, for example, the severity or persistence of the disease or condition in the patient. The chelating agent can be delivered to the patient for a time (including a protracted period, e.g., several months or years) sufficient to treat the condition and exert the intended pharmacological or biological effect.

### Screening Methods

The present invention also provides a method for identifying or isolating a compound for preventing or treating obesity and/or associated insulin resistance and/or increasing metabolism and/or reducing adiposity in a subject, said method comprising determining the ability of the compound to increase HIF-1α expression or activity in a cell or in a tissue or organ of an animal, wherein increased expression or activity of HIF-1α indicates that the compound prevents or treats obesity and/or associated insulin resistance and/or increasing metabolism and/or reducing adiposity in a subject.

Preferably, the method additionally comprises:

(i) administering the compound to a subject suffering from or developing obesity, insulin resistance, reduced metabolism or increased adiposity and assessing obesity, insulin resistance, metabolism or adiposity in said subject; and
(ii) comparing the obesity, insulin resistance, metabolism or adiposity in said subject at (i) to the level in a subject suffering from or developing obesity, insulin resistance, reduced metabolism or increased adiposity to which the compound has not been administered,
wherein reduced obesity, reduced insulin resistance, increased metabolism or reduced adiposity in the subject at (i) compared to (ii) indicates that the compound prevents or treats obesity and/or associated insulin resistance and/or increasing metabolism and/or reducing adiposity in a subject.

This invention also provides for the provision of information concerning the identified or isolated compound. Accordingly, the screening assays are further modified by:

(i) optionally, determining the structure of the compound; and
(ii) providing the compound or the name or structure of the compound such as, for example, in a paper form, machine-readable form, or computer-readable form.

Naturally, for compounds that are known albeit not previously tested for their function using a screen provided by the present invention, determination of the structure of the compound is implicit. This is because the skilled artisan will be aware of the name and/or structure of the compound at the time of performing the screen.

As used herein, the term “providing the compound” shall be taken to include any chemical or recombinant synthetic means for producing said compound or alternatively, the provision of a compound that has been previously synthesized by any person or means. This clearly includes isolating the compound.

In a preferred embodiment, the compound or the name or structure of the compound is provided with an indication as to its use e.g., as determined by a screen described herein.

The screening assays can be further modified by:

(i) optionally, determining the structure of the compound;
(ii) optionally, providing the name or structure of the compound such as, for example, in a paper form, machine-readable form, or computer-readable form; and
(iii) providing the compound.

In a preferred embodiment, the synthesized compound or the name or structure of the compound is provided with an indication as to its use e.g., as determined by a screen described herein.

The present invention is described further in the following non-limiting examples.

### EXAMPLES

### Example 1

### Materials and Methods

### Animals

Four studies were performed using C57Bl/6 mice. Study 1 included 30 control mice and 30 mice administered DFS. Study 2 included 30 control mice and 30 mice administered DFS. Study 3 included 8 control mice and 8 mice administered DFS. Study 4 included 18 control mice and 18 mice administered DFS. Mice in Study 3 differed to those in studies 1 and 2 in so far as they were sourced from different colonies.

Studies were also performed using ob/ob mice, in particular, 5 ob/ob controls and 5ob/ob mice administered DFS. Wild type C57bl/6 mice were also used as controls, 4 untreated and 4 administered DFS.

### Antibodies

Anti-HIF-1α antibody was purchased from Novus Biologicals (Littleton, Colo.). Anti-mouse Ig HRP conjugated antibody was purchased from Santa Cruz (Santa Cruz, Calif.).

### High-Fat Diet and Iron Chelation Therapy.

Mice were randomly separated equally into a treatment group receiving Deferasirox (DFS) or a control group (CON). DFS was powdered using a mortar and pestle and mixed in thoroughly with the dry mineral mix prior to the addition of melted lard and then left to harden overnight in the refrigerator to form high-fat diet (HFD). From 6 weeks of age for up to 25 weeks, the mice were then fed ad libitum this HFD, from which 45% of their calories were derived from animal lard.

### In-Vivo Studies.

Each mouse was weighed weekly and the blood glucose levels assessed at random times every second week alternating with rectal temperatures. Intraperitoneal Glucose Tolerance Testing (IPGTT) was carried out at week 5 and 21 using 2g glucose/kg dose. Insulin Tolerance Testing (ITT) was carried at week 2 (0.33 U/kg) and week 6 (0.50 U/kg). Glucose Stimulated Insulin Secretion (GSIS)(3g glucose/kg) was studied out at week 7. Food intake studies were carried out at weeks 0, 4, 8 and 25. Indirect calorimetry was performed using the Oxymax System (Columbus Instruments, Columbus, Ohio) at weeks 0, 4, 8 and 25. Measurements were taken over a 12-hour light cycle and a 12-hour dark cycle.

### Glucose Tolerance Tests

Mice were fasted overnight for 16 hours. Glucose was administered at a dose of 2g/kg by intraperitoneal injection in the form of a 20% dextrose solution. Blood glucose was measured via glucometer (Accucheck Advantage II, Roche, Australia) prior to, and at 15, 30, 60, 90 and 120 minutes after the dextrose injection.

### Insulin Tolerance Tests

Mice were fasted overnight for 16 hours. Insulin was injected at 0.5 units per kg (diluted in 1× PBS with 1% bovine serum albumin) by intraperitoneal injection. Blood glucose was measured via glucometer prior to, and at 10, 20, 30, 45 and 60 minutes after the insulin injection.

### Histological Preparation.

Mice livers were fixed in 10% buffered formaldehyde, embedded in paraffin and slides were prepared using standard haematoxylin and eosin as well as Perl staining to visualize hepatic iron.

### Immunoprecipitation

Indicated cell lysates were incubated with 2 μg anti-HIF-1α antibody overnight. HIF-1 α immune complexes were collected using protein A-G sepharose beads. Precipitates were washed in cell lysis buffer, and proteins eluted with reducing sample buffer. Proteins were separated by 10% SDS-PAGE and transferred to PVDF membrane. The membrane was blotted with PBST with 5% milk, followed by the anti-HIF-1 α antibody (above) and subsequently anti-mouse HRP-conjugated secondary antibody. Proteins were visualised using enhanced chemiluminescence.

### Indirect Calorimetry

Mice were placed with ad libitum food and water in separate chambers in an 8 chamber, indirect open circuit calorimeter (Columbus Oxymax Respirometer 0246-002M, Columbus Instruments Ohio USA) for 36-48 hours. This machine obtains periodic measurements of the percentage of oxygen and carbon dioxide in the test chamber. Changes in gas concentrations are used to calculate the rate of oxygen consumption (VO₂) and carbon dioxide production (VCO₂) per mouse, with a readout every 27 minutes (Oxymax Software 0246-102M). The software calculates heat production per mouse and the respiratory exchange ratio (RER) (VCO₂/VO₂) using VO₂ and VCO₂. Activity levels were simultaneously measured in 2 dimensions (Opto-M3 activity meter) using 875 nM wavelength light beams. Ambulatory movements were registered when more than one light beams in the same axis were crossed by an animal in a given period of time. The last 24 hours of data were analysed to allow for an acclimatisation period.

### Analysis of Indirect Calorimetry Data

The hourly mean VO₂ and VCO₂ (mL/hr/kg total body weight) were calculated by averaging the VO₂ and VCO₂ readings produced by the Oxymax software for each hour. VO₂ and VCO₂ per unit of lean body mass (VXX_{2LM}) was calculated from the Oxymax results (VXX₂) using the following equation:

VXX_{2LM}=(VXX₂(mL/hr/kg)*total body weight (kg))/lean body mass (kg).

Mean total, light and dark VXX_{2LM} were calculated by obtaining the mean hourly readings for a 24 hour period, light hours (0700-1900 h) and dark hours (1900-0700 h) respectively.

The hourly mean RER and the total, light and dark RER were calculated in a similar manner to VO₂ and VCO₂. No adjustment for body weight was required.

Heat (total energy expenditure, kcal/hr/mouse) was calculated by the Oxymax software using the formula:

Heat=CV (calorific value)*VO₂ where CV=3.815+(1.232*RER)

The hourly mean heat production and the total, light and dark heat production were calculated in a similar manner to VO₂ and VCO₂. Heat production per unit of lean body mass was calculated by dividing the Oxymax reading by unit of lean body mass.

To quantify activity levels, the total number of ambulatory movements in the x axis and the y axis was calculated per unit time for each mouse.

Oxymax data were analysed using t-tests.

### Plasma and Liver Biochemistry, and Lipid Parameters.

Plasma insulin was measured with ELISA kits. Liver transaminases and plasma lipids were performed by Sydpath Pathology, St Vincent's Hospital. Liver triglycerides were extracted using methanol/chloroform 2:1 and detected using colorimetric assay.

### Protein Extraction and Immunoblotting.

Fresh tissues were removed rapidly from culled animals and kept frozen in the −80° C. freezer. They were then homogenized in lysis buffer and sonicated in cold RIPA buffer (50 nM Tris pH 7.4, 1% NP40, 0.25% sodium deoxycholate, 150 nM NaCl, 1 mM EDTA, 1 mM PMSF and protease inhibitor cocktail). 121 μg of protein sample was resolved on an 8% SDS PAGE gel. Proteins were transferred onto nitrocellulose membrane. Primary antibodies (HIF-1α, and (β-tubulin) were prepared in milk buffers at concentrations of 1:500 and 1:2000 respectively and the membrane was incubated overnight at 4° C. Membranes were then rinsed with Phosphate Buffered Solution containing Tween and HRP-conjugated secondary antibodies (at 1:1000 dilution). Proteins were visualised using enhanced chemiluminescence.

### RNA Preparation and Quantitative Real-Time PCR.

Tissues were homogenized in extraction buffer and RNA was isolated according to the RNeasy Kit protocol. First strand cDNA was achieved with Superscript enzymes kit and cDNA amplification performed using SybrGreen via ABI 7900 qPCR Sequence Detection System according to protocols provided by the manufacturer (Applied Biosystems). The level of mRNA for each gene was normalized to the level of TATA box binding-protein (TBP) mRNA in each sample.

### Calculations.

Data are expressed as means+SEM. Statistical analysis was conducted using Student's t test. Statistical significance defined as P<0.05.

### Glucose Tolerance Test

Mice were fasted for 6 hours before intraperitoneal administration of glucose (2 g/kg dose) in sterile water. Blood glucose was measured by glucometer at the times indicated.

### Example 2

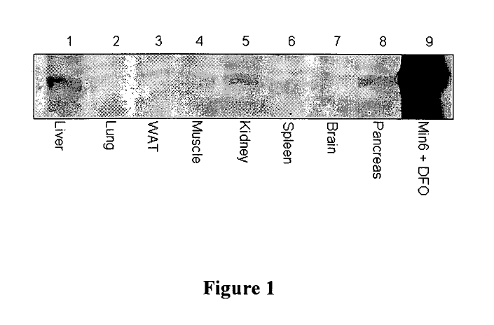

HIF-1 α protein is detectable in muscle. Immunoprecipitation studies were performed on a range of mouse tissues as shown in FIG. 1. Muscle was shown to express significant amounts of HIF-1α protein (Lane 4), consistent with previous findings.

### Example 3

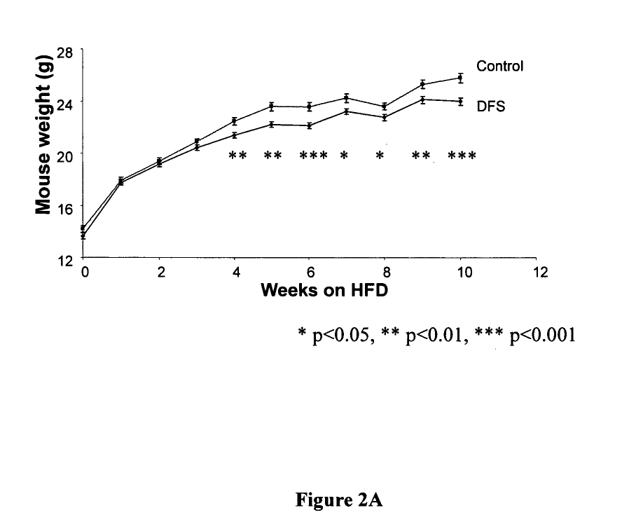

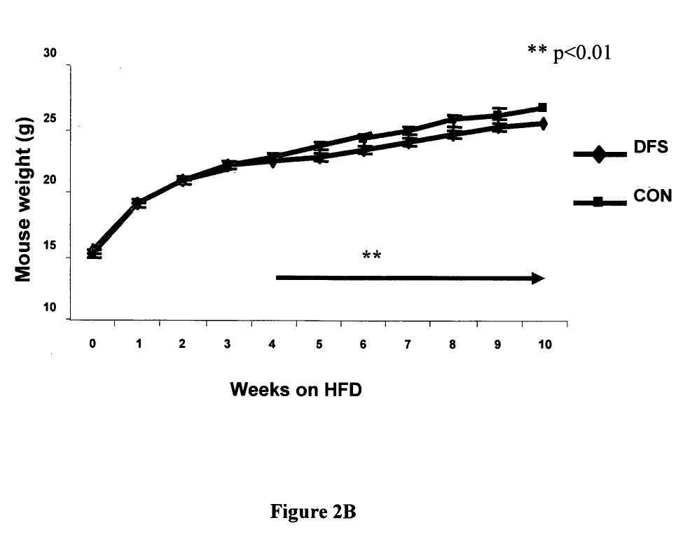

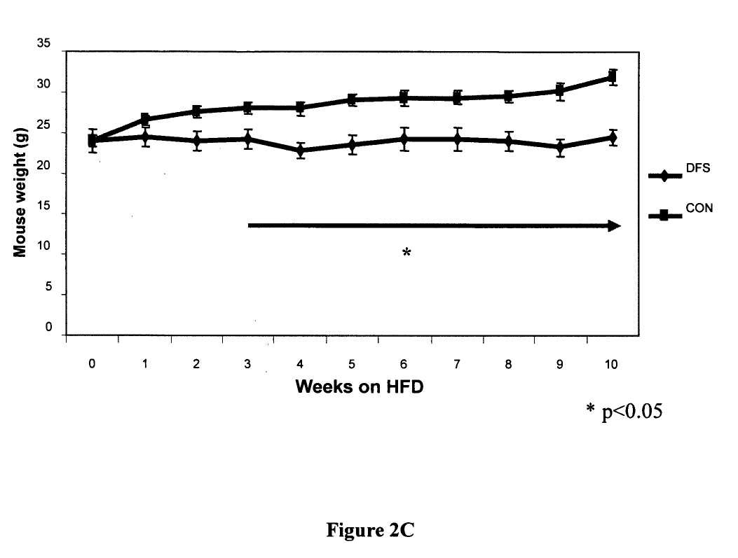

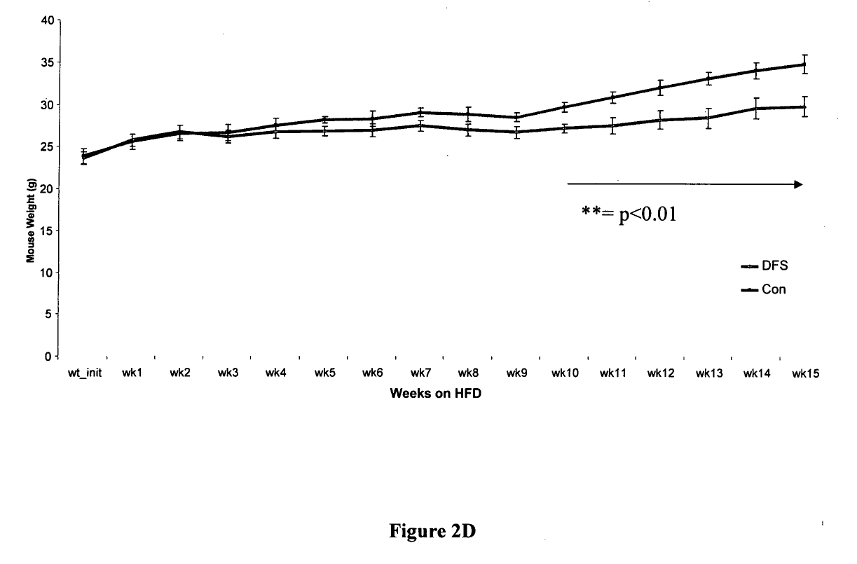

Mice treated with DFS are resistant to high fat induced obesity: As shown in FIGS. 2A-D mice treated with DFS in Studies 1-4 had significantly reduced body weights compared to control mice from between about 3 weeks and about 10 weeks after commencement of a high fat diet.

### Example 4

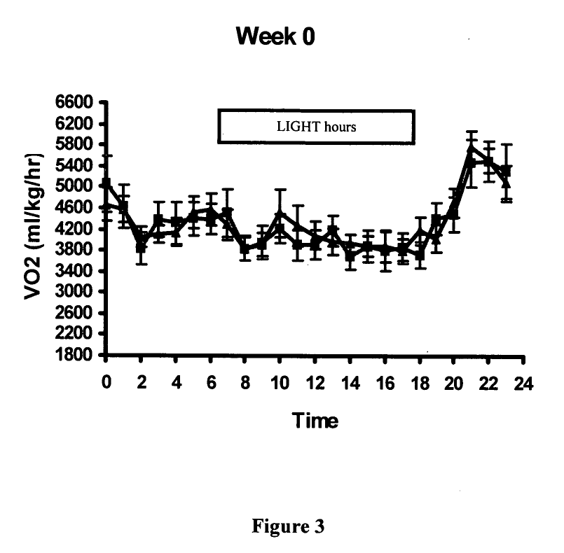

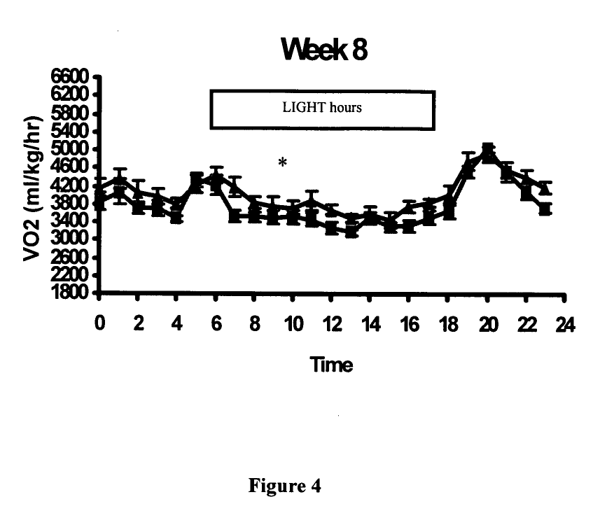

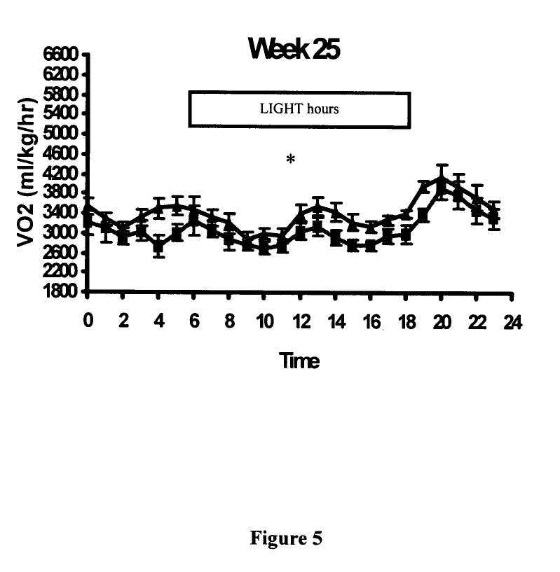

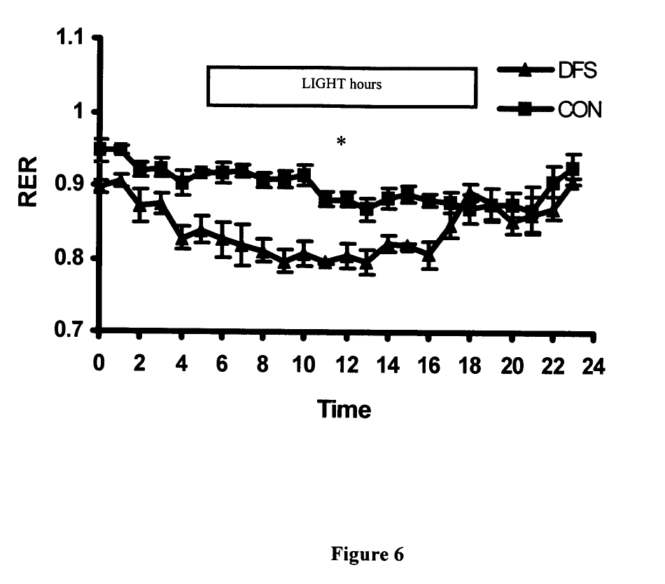

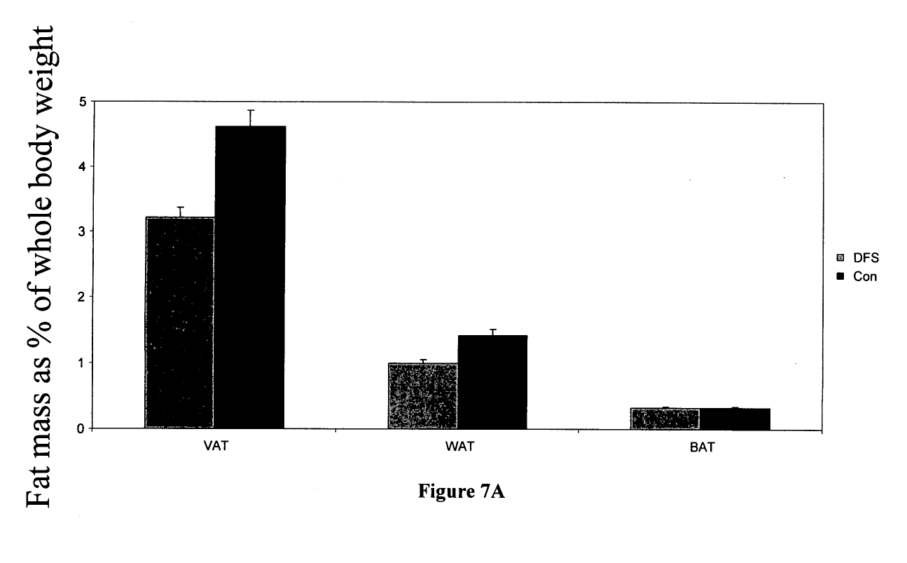

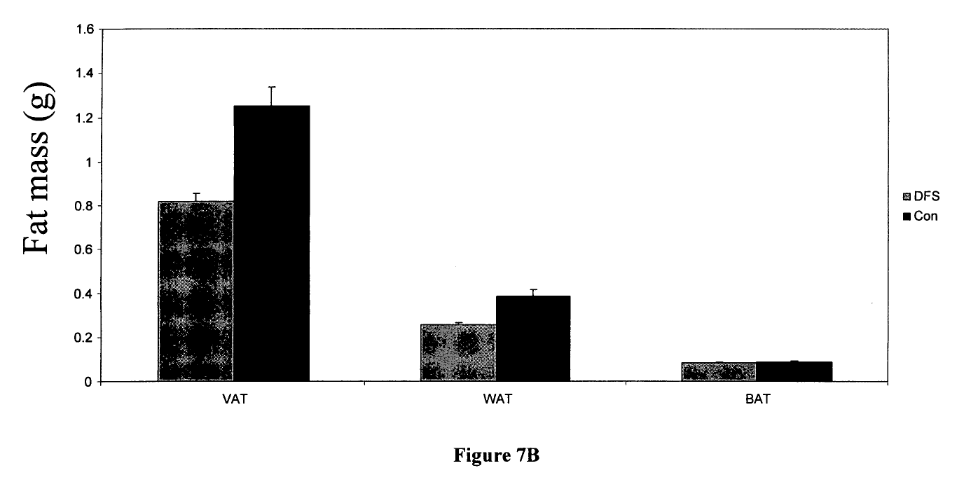

DFS treated mice have improved whole body metabolism and energy expenditure. Baseline metabolic rates of the DFS treated and CON mice were similar at week 0 (FIG. 3). By week 8 (FIG. 4) and week 25 (FIG. 5) of continuous DFS treatment, the treated mice had significantly increased O₂ consumption and CO₂ production, suggesting improved whole body metabolism. In addition, the DFS mice had reduced respiratory exchange ratios (RER) (FIG. 6) consistent with preferential fat utilization. Consistent with these results is the demonstration that the weight of white adipose tissue and visceral fat is reduced in DFS treated animals compared to CON animals, whereas the weight of brown adipose tissues (associated with energy consumption and heat production) is unchanged (FIGS. 7A and B).

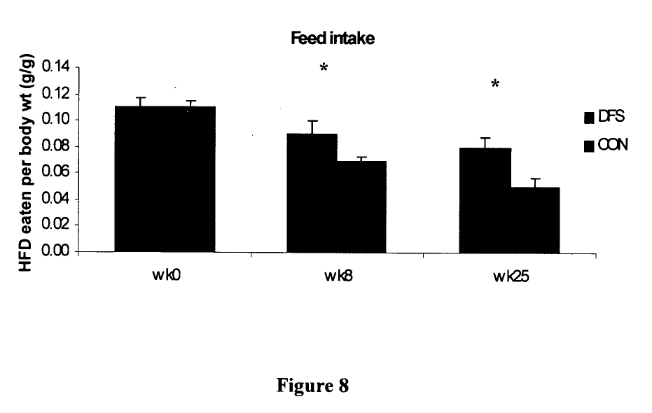

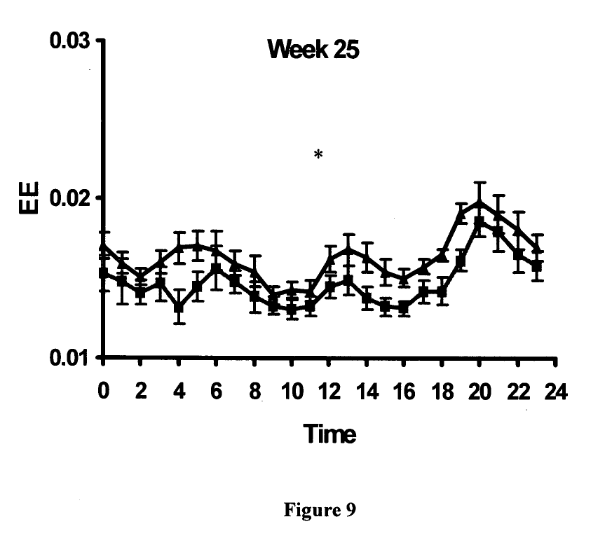

Feed intake did not differ initially (week 0) between DFS treated animals and CON animals. However by 25 weeks, it appeared that DFS treated mice had eaten more HFD adjusted per body weight, compared with the CON mice (FIG. 8). In addition, it was observed that the DFS mice have significantly higher energy expenditure (EE) (FIG. 9). Taken together these data indicate that the DFS treated mice have a significantly higher metabolic rate than control mice. These observations were not dependent on activity level, as the DFS mice, in general, were less active than the CON mice (data not shown).

### Example 5

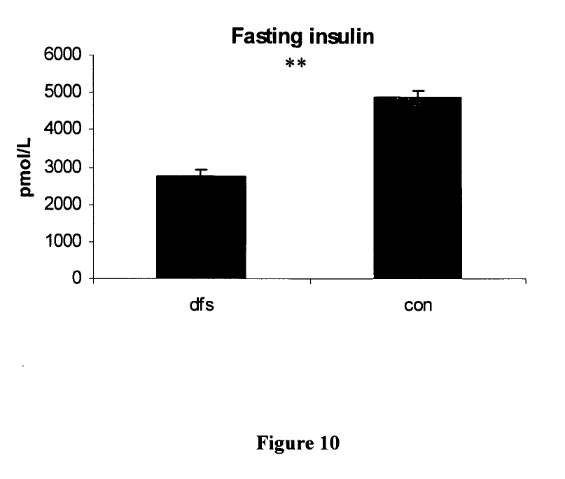

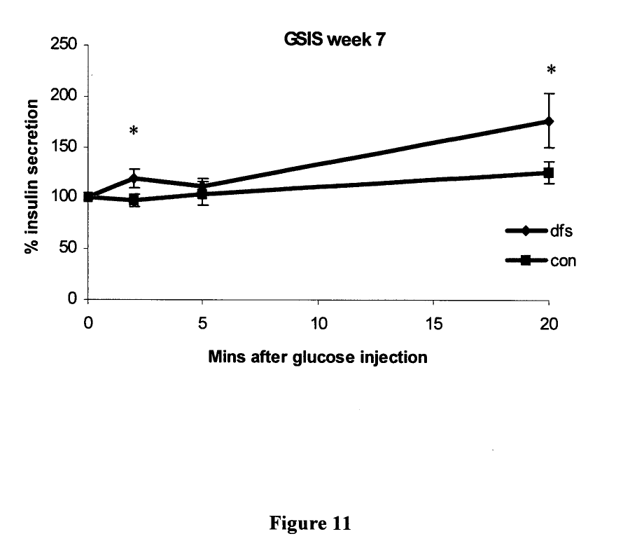

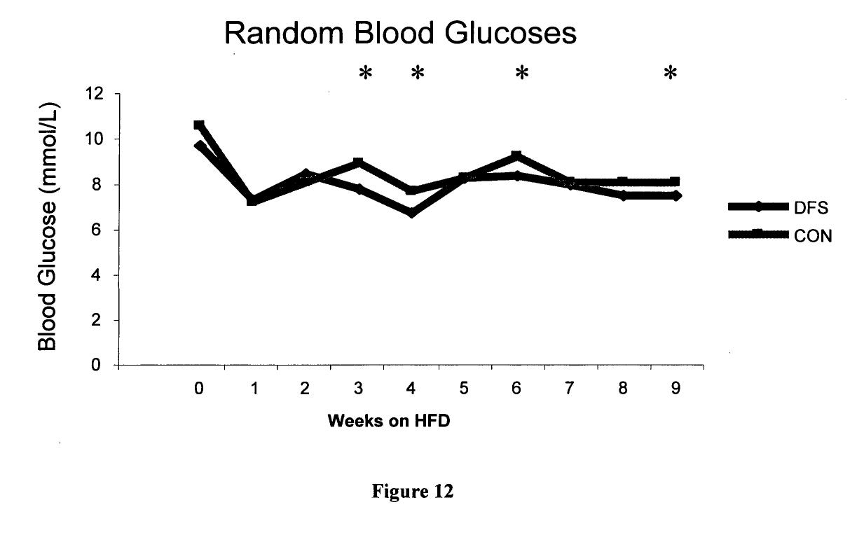

DFS treated mice are resistant to obesity induced insulin resistance. Treated animals had significantly lower fasting insulin levels (2837+874 pmol/L vs 4703+1741 pmol/L) (p=0.005) (FIG. 10) and better preservation of glucose stimulated insulin secretion (GSIS) profile (FIG. 11). Furthermore, blood glucose testing at random times showed that mice treated with DFS generally had lower blood glucose levels that CON mice (FIG. 12).

About 80% of the treated animals had glucoses <2 mmol/L compared with 38% of controls (p<0.001) indicating improved insulin sensitivity (data not shown).

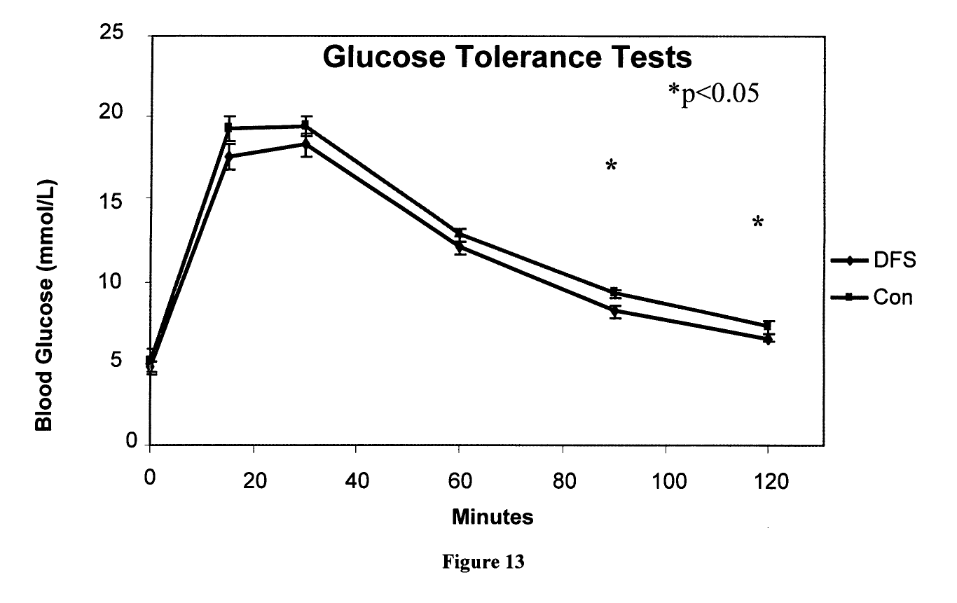

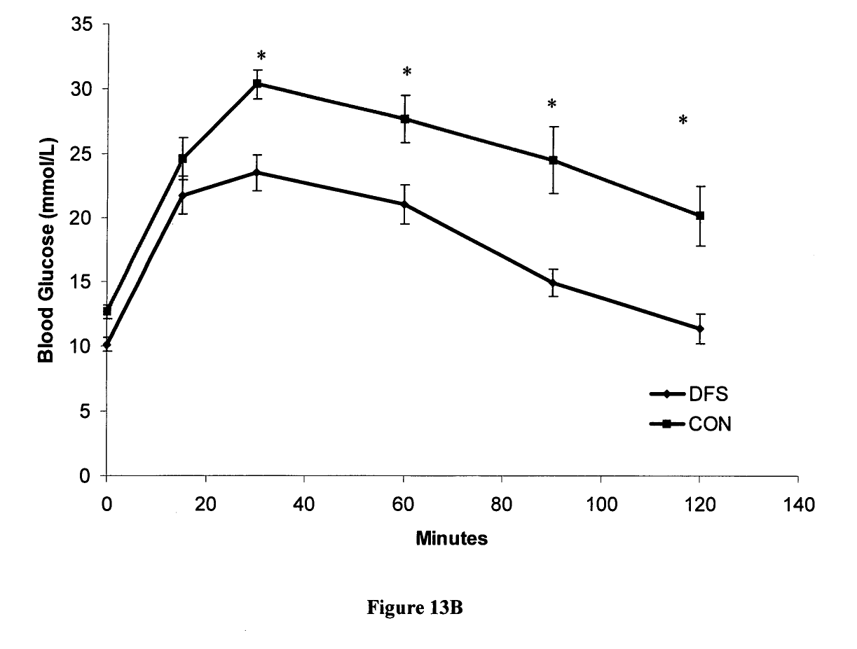

By week 5, DFS treated mice demonstrated significantly better glucose tolerance when compared with CON mice and this continued beyond at least week 21 (FIGS. 13A and B).

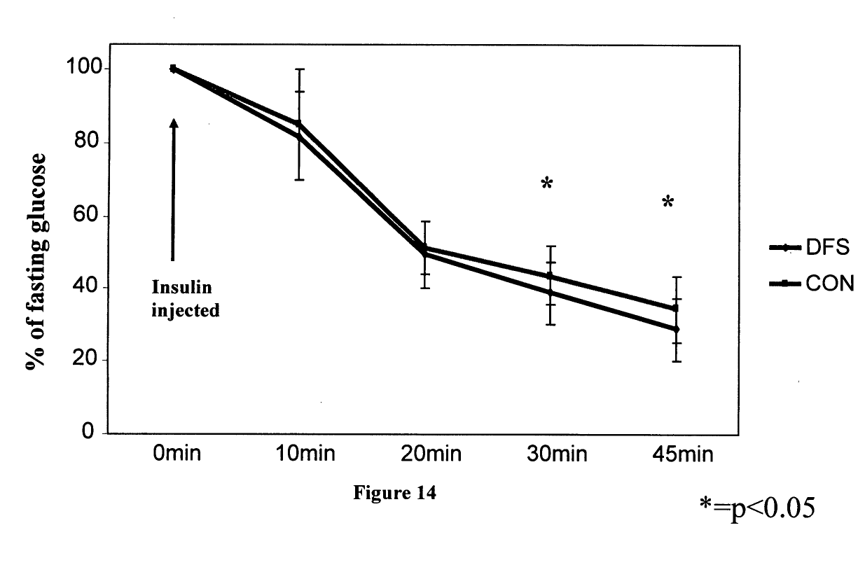

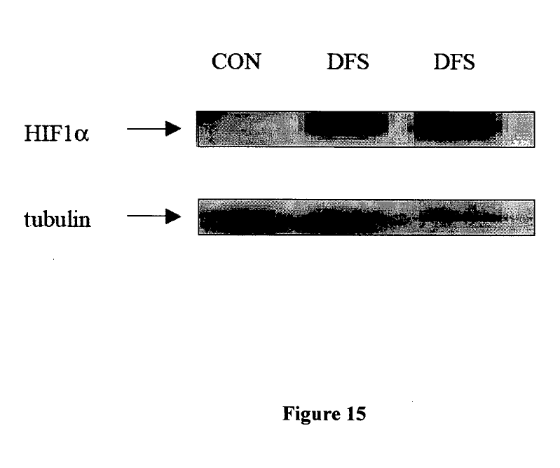

Furthermore, DFS mice performed significantly better than CON mice in insulin tolerance tests (ITTs) (FIG. 14). These data indicate that DFS treatment improves pituitary function and/or adrenal function in so far as it improves a subject's response to insulin-induced hypoglycaemia.

### Example 6

DFS treated mice are resistant to HFD-induced hepatic lipid accumulation. HFD has been associated with hepatic steatosis. By 10 weeks of being on HFD, CON mice have developed macroscopic steatosis, while the DFS mice are relatively protected (data not shown).

### Example 7

DFS treated livers have lower iron stores and show increased levels of HIF-1α. Because DFS is known to be an effective oral tissue iron chelator, iron levels in the liver of treated and control mice was tested. Livers of DFS treated mice have lower iron levels compared to CON mice (data not shown).

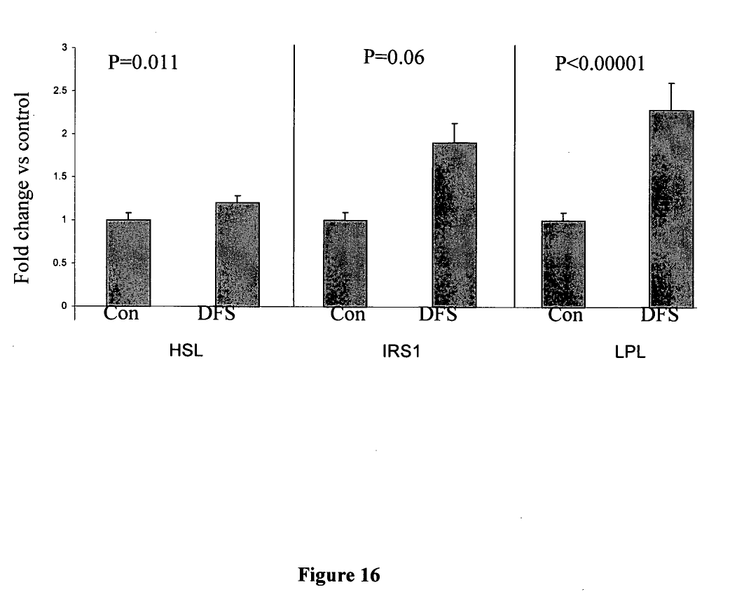

Without been bound by theory or mode of action, the present inventors consider that HIF-1α protein degradation by the von Hippel-Lindau (VHL) protein is effected by intracellular iron, and that it is likely that the reduction of hepatic iron will increase liver HIF-1α protein. Western blots of liver lysates demonstrates increased HIF-1α in DFS treated mice (FIG. 16).

### Example 8

DFS treated livers have increased gene expression for insulin signaling and lipid metabolic pathways. Because HIF-1α binds with the ubiquitous aryl hydrocarbon receptor nuclear translocator (ARNT) to transcribe molecules important for insulin signaling and glycolysis pathways. The increase in HIF-1α in DFS treated mice was associated with significantly increased gene expression for AKT2, IRS1, IRS2, PFK. There was a trend for improved GLUT2 expression in the liver. In addition, hormone sensitive lipase (HSL) and lipoprotein lipase (LPL) were also significantly increased in livers of DFS treated mice (FIG. 16). Without being bound by any theory or mode of action, these data together with the metabolic observations discussed above, may reflect increased lipolysis, thus explaining the weight protection that DFS affords the treated mice.

### Example 9

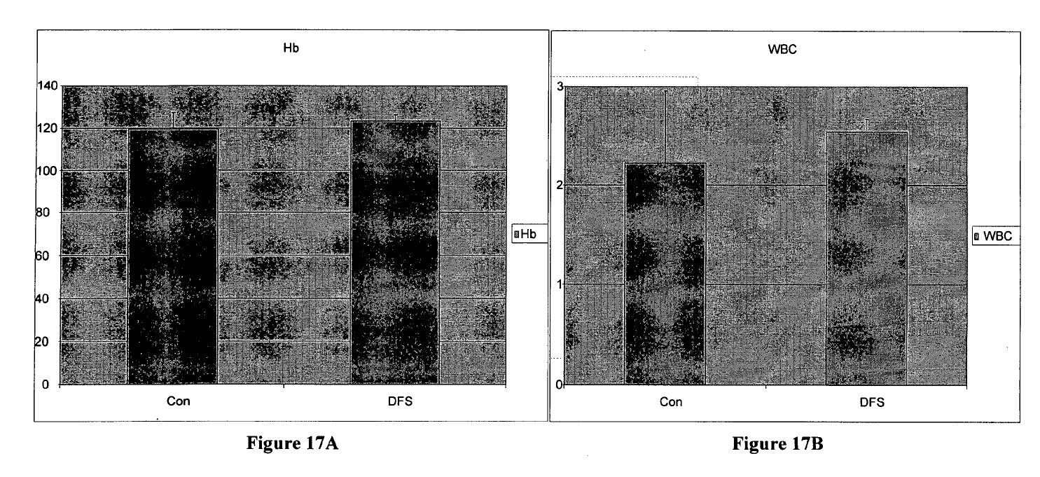

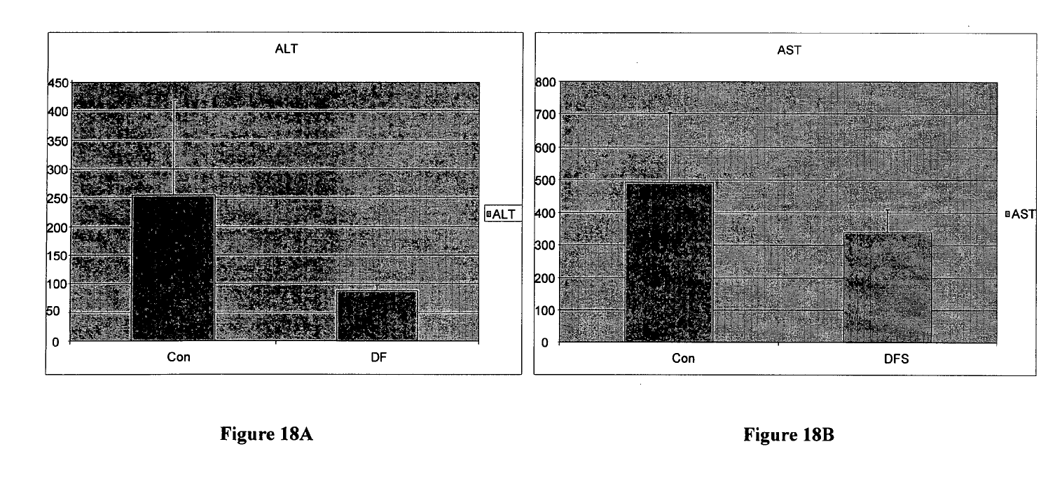

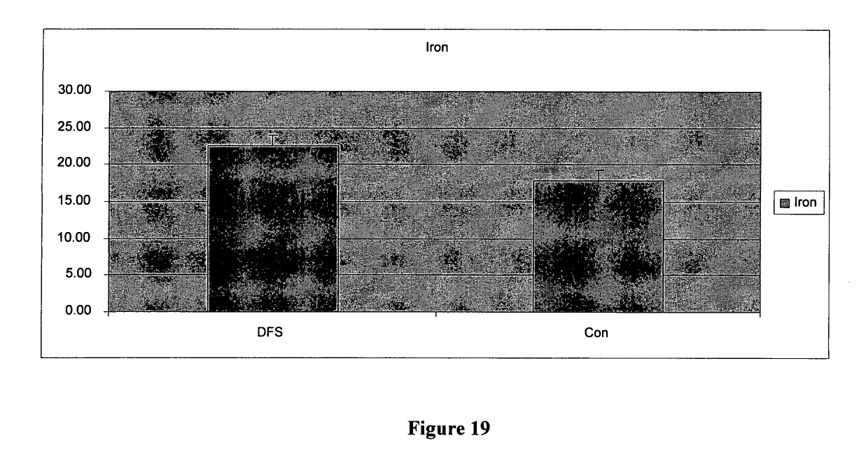

DFS treatment does not appear to induce liver or blood dysfunction. Given that DFS is an iron chelator and results in biological changes in the liver of a treated subject, it was important to determine whether or not treatment with this compound results in adverse reactions. As shown in FIGS. 17-19, DFS treatment does not result in anaemia, or significantly reduced serum iron levels or liver function.

### DFS Treatment Reduces Weight in Obese Mice

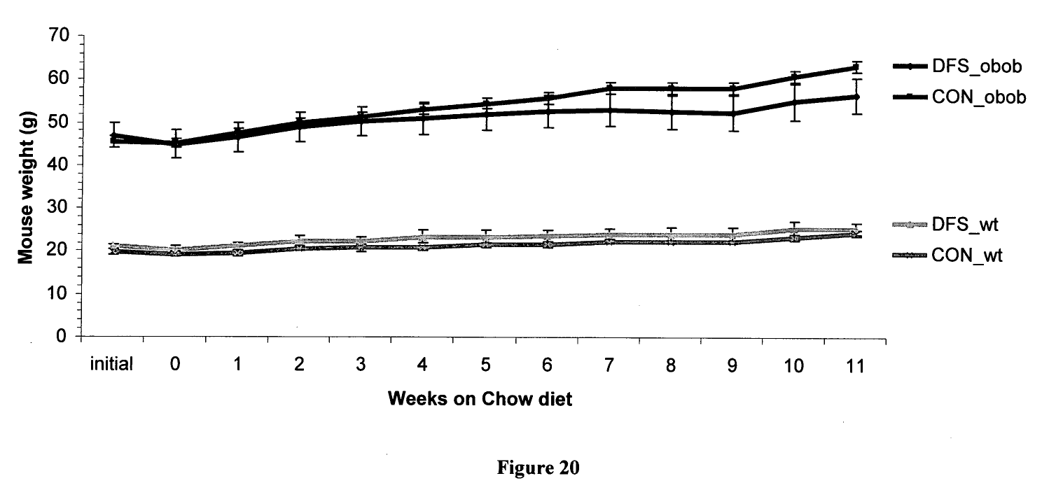

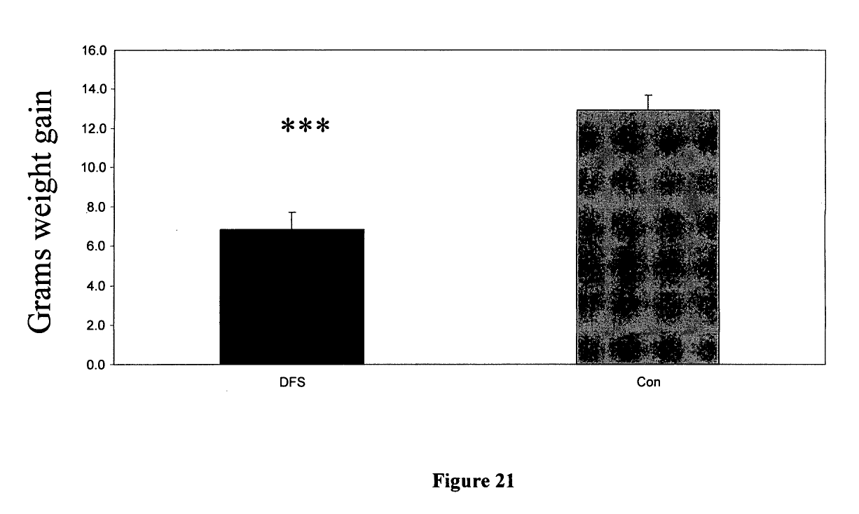

DFS treatment of obese mice (ob/ob) fed on a normal chow diet results in reduced body weight (or reduced body weight increase) compared to control ob/ob mice (FIG. 20). As shown in FIG. 22, the weight gained over an eight week period in DFS ob/ob mice is significantly lower than the weight gained in control ob/ob mice.

### REFERENCES

Almeida and Allshire (2005), TRENDS Cell Biol., 15:251-258.
Briggs et al. (1986), Science, 234:47-52.
Deshpande et al. (1997), J. Biol. Chem., 272(16):10664-10668.
Harayama (1998), Trends Biotechnol., 16: 76-82.
Haseloff and Gerlach (1988), Nature, 334:585-591.
Hotamisligil et al. (1993), Science, 259: 87-90.
Klein et al. (1998), Exp. Neurol., 150:183-194.
Li et al. (1999), Nat. Biotechnol., 17(3):241-245.
Millar and Waterhouse (2005), Funct. Integr. Genomics, 5:129-135.
Mitchell and Tjian (1989), Science, 245:371-378.
Needleman and Wunsch (1970), J. Mol. Biol., 48:443-453.
Nettelbeck et al. (1998), Gene Ther., 5(12)1656-1664.
Pasquinelli et al. (2005), Curr. Opin. Genet. Develop., 15:200-205.
Perriman et al. (1992), Gene, 113:157-163.
Pitluk et al. (1991), J. Virol., 65:6661-6670.
Shippy et al. (1999), Mol. Biotech., 12:117-129.
Smith et al. (2000), Nature, 407:319-320.
Stewart et al. (1996), Genomics, 37(1):68-76.
Waterhouse et al. (1998), Proc. Natl. Acad. Sci. USA, 95:13959-13964.
Zolotukiin et al. (1996), J. Virol., 70(7):4646-4654.

1. A method for increasing metabolism and/or energy expenditure in a subject, the method comprising administering an iron chelating agent to the subject.

2. The method according to claim 1, wherein the iron chelator is a compound of formula (I): 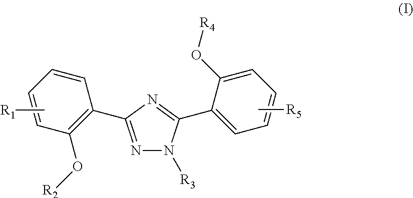
in which
R₁ and R₅ simultaneously or independently of one another are hydrogen, halogen, hydroxyl, lower alkyl, halo-lower alkyl, lower alkoxy, halo-lower alkoxy, carboxyl, carbamoyl, N-lower alkylcarbamoyl, N,N-di-lower alkylcarbamoyl or nitrile; R₂ and R₄ simultaneously or independently of one another are hydrogen, unsubstituted or substituted lower alkanoyl or aroyl, or a radical which can be removed under physiological conditions; R₃ is hydrogen, lower alkyl, hydroxy-lower alkyl, halo-lower alkyl, carboxy-lower alkyl, lower alkoxycarbonyl-lower alkyl, R₆R₇N—C(O)-lower alkyl, unsubstituted or substituted aryl or aryl-lower alkyl, or unsubstituted or substituted heteroaryl or heteroaralkyl; R₆ and R₇ simultaneously or independently of one another are hydrogen, lower alkyl, hydroxy-lower alkyl, alkoxy-lower alkyl, hydroxyalkoxy-lower alkyl, amino-lower alkyl, N-lower alkylamino-lower alkyl, N,N-di-lower alkylamino-lower alkyl, N-(hydroxy-lower alkyl)amino-lower alkyl, N,N-di(hydroxy-lower alkylamino-lower alkyl or, together with the nitrogen atom to which they are bonded, form an azaalicyclic ring; or a pharmaceutically acceptable salt thereof.

3. (canceled)

4. The method according to claim 1, wherein the increased metabolism is or includes increased fat metabolism.

5. The method according to claim 1, wherein the increase in metabolism in the subject reduces adiposity in the subject and/or prevents an increase in adiposity in the subject and/or treats or prevents obesity or associated insulin resistance in the subject.

6. A method for increasing metabolism and/or energy expenditure in a subject, the method comprising increasing the level and/or activity of Hypoxia Induced Factor 1α (HIF-1α) in a cell, tissue or organ of the subject, increasing metabolism in the subject.

7. The method according to claim 6, wherein the increased metabolism is or includes increased fat metabolism.

8. The method according to claim 6, wherein the increase in metabolism in the subject reduces adiposity in the subject and/or prevents an increase in adiposity in the subject and/or treats or prevents obesity or associated insulin resistance in the subject.

9. The method according to claim 6, wherein the cells of the subject are adipocytes and/or skeletal muscle cells and/or cells of the nervous system involved in regulation of energy intake and energy expenditure and/or hepatocytes and/or the tissue is fat and/or skeletal muscle and/or neural tissue and/or liver tissue.

10. The method according to claim 6 wherein the level and/or activity of HIF-1α is increased by administering to the subject a compound that increases the level and/or activity of HIF-1α in a cell, tissue or organ thereof.

11. The method according to claim 10, wherein the compound increases stability and/or reduces degradation of HIF-1α in a cell, tissue or organ of the subject thereby resulting in an increased levels and/or activity of HIF-1α in the cell, tissue or organ.

12. The method according to claim 11, wherein the compound reduces degradation of HIF-1α by inhibiting or completely inhibiting or preventing activity of a protein that mediates degradation of HIF-1α.

13.-15. (canceled)

16. The method according to claim 6, wherein the level or activity of HIF-1α is increased by administering to the subject a chelating agent.

17. The method according to claim 16, wherein the chelating agent is an iron chelating agent.

18. The method according to claim 17, wherein the iron chelating agent is a bidentate iron chelating agent or a tridentate iron chelating agent or a higher order multidentate iron chelating agent or a non-naturally occurring iron chelating agent.

19. The method according to claim 17, wherein the iron chelating agent is selected individually or collectively from the group consisting of 4-[3,5-Bis (2-hydroxyphenyl)-1H-1,2,4-triazol-1-yl]-benzoic acid, N-(5-C3-L (5 aminopentyl)hydroxycarbamoyl)-propionamido)pentyl)-3(5-(N-hydroxyactoamido)-pentyl)carbamoyl)-propionhydroxamic acid, 2-deoxy-2-(N-carbamoylmethyl-[N′-2′-methyl-3′-hydroxypyridin-4′-one])-D-glucopyranose, pyridoxal isonicotinyl hydrazone, 4,5-dihydro-2-(2,4-dihydroxyphenyl)-4-methylthiazole-4-carboxylic acid, 4,5-dihydro-2-(3′-hydroxypyridin-2′-yl)-4-methylthiazole-4-carboxylic acid, 4-[3,5-bis(2-hydroxyphenyl)-[1,2,4]triazol-1-yl]benzoic acid, N,N′-bis(o-hydroxybenzyl)ethylenediamine-N,N′-diacetic acid), ferrioxamine, trihydroxamic acid, EDTA, desferrioxamine hydroxamic acids, deferoxamine B as a methanesulfonate salt, apoferritin, trans-1,2-diaminocyclohexane-N,N,N′,N′-tetraacetic acid, diethylenetriamine-N,N,N′,N″,N″-penta-acetic acid, 1,2 dimethyl-3-hydroxypyridin-4-one, a cobaltous ion, a non-crystal form of any of the foregoing, a crystal form of any of the foregoing, a salt of any of the foregoing, a derivative of any of the foregoing and mixtures thereof.

20. The method according to claim 17, wherein the iron chelating agent is a 3,5-diphenyl-1,2,4-triazole or a salt thereof or a crystalline form thereof.

21. The method according to claim 20, wherein the compound is 4-[3,5-Bis (2-hydroxyphenyl)-1H-1,2,4-triazol-1-yl]-benzoic acid or a salt thereof or a crystalline form thereof.

22. The method according to claim 10, wherein the level and/or activity of HIF-1α is increased by administering to the subject a compound that increases HIF-1α expression.

23.-27. (canceled)

28. The method according to claim 1, wherein the compound or agent is administered a plurality of times to a subject.

29. The method according to claim 1, wherein the compound or agent is administered in the form of a pharmaceutical composition additionally comprising a pharmaceutically acceptable carrier and/or diluent.

30.-35. (canceled)

36. The method according to claim 1, wherein the iron chelating agent is 4-[3,5-Bis (2-hydroxyphenyl)-1H-1,2,4-triazol-1-yl]-benzoic acid or a salt thereof.


SEQUENCE LISTING (26 sequences)

SEQ ID NO: 1 - protein, 826 aa, Homo sapiens
MEGAGGANDKKKISSERRKEKSRDAARSRRSKESEVFYELAHQLPLPHNVSSHLDKASVMRLTISYLRVRKLLDAGDLDIEDDMKAQMNCFYLKALDGFVMVLTDDGDMIYISDNVNKYMGLTQFELTGHSVFDFTHPCDHEEMREMLTHRNGLVKKGKEQNTQRSFFLRMKCTLTSRGRTMNIKSATWKVLHCTGHIHVYDTNSNQPQCGYKKPPMTCLVLICEPIPHPSNIEIPLDSKTFLSRHSLDMKFSYCDERITELMGYEPEELLGRSIYEYYHALDSDHLTKTHHDMFTKGQVTTGQYRMLAKRGGYVWVETQATVIYNTKNSQPQCIVCVNYVVSGIIQHDLIFSLQQTECVLKPVESSDMKMTQLFTKVESEDTSSLFDKLKKEPDALTLLAPAAGDTIISLDFGSNDTETDDQQLEEVPLYNDVMLPSPNEKLQNINLAMSPLPTAETPKPLRSSADPALNQEVALKLEPNPESLELSFTMPQIQDQTPSPSDGSTRQSSPEPNSPSEYCFYVDSDMVNEFKLELVEKLFAEDTEAKNPFSTQDTDLDLEMLAPYIPMDDDFQLRSFDQLSPLESSSASPESASPQSTVTVFQQTQIQEPTANATTTTATTDELKTVTKDRMEDIKILIASPSPTHIHKETTSATSSPYRDTQSRTASPNRAGKGVIEQTEKSHPRSPNVLSVALSQRTTVPEEELNPKILALQNAQRKRKMEHDGSLFQAVGIGTLLQQPDDHAATTSLSWKRVKGCKSSEQNGMEQKTIILIPSDLACRLLGQSMDESGLPQLTSYDCEVNAPIQGSRNLLQGEELLRALDQVN

SEQ ID NO: 2 - protein, 735 aa, Homo sapiens
MEGAGGANDKKKISSERRKEKSRDAARSRRSKESEVFYELAHQLPLPHNVSSHLDKASVMRLTISYLRVRKLLDAGDLDIEDDMKAQMNCFYLKALDGFVMVLTDDGDMIYISDNVNKYMGLTQFELTGHSVFDFTHPCDHEEMREMLTHRNGLVKKGKEQNTQRSFFLRMKCTLTSRGRTMNIKSATWKVLHCTGHIHVYDTNSNQPQCGYKKPPMTCLVLICEPIPHPSNIEIPLDSKTFLSRHSLDMKFSYCDERITELMGYEPEELLGRSIYEYYHALDSDHLTKTHHDMFTKGQVTTGQYRMLAKRGGYVWVETQATVIYNTKNSQPQCIVCVNYVVSGIIQHDLIFSLQQTECVLKPVESSDMKMTQLFTKVESEDTSSLFDKLKKEPDALTLLAPAAGDTIISLDFGSNDTETDDQQLEEVPLYNDVMLPSPNEKLQNINLAMSPLPTAETPKPLRSSADPALNQEVALKLEPNPESLELSFTMPQIQDQTPSPSDGSTRQSSPEPNSPSEYCFYVDSDMVNEFKLELVEKLFAEDTEAKNPFSTQDTDLDLEMLAPYIPMDDDFQLRSFDQLSPLESSSASPESASPQSTVTVFQQTQIQEPTANATTTTATTDELKTVTKDRMEDIKILIASPSPTHIHKETTSATSSPYRDTQSRTASPNRAGKGVIEQTEKSHPRSPNVLSVALSQRTTVPEEELNPKILALQNAQRKRKMEHDGSLFQAVGII

SEQ ID NO: 3 - protein, 836 aa, Mus musculus
MEGAGGENEKKKMSSERRKEKSRDAARSRRSKESEVFYELAHQLPLPHNVSSHLDKASVMRLTISYLRVRKLLDAGGLDSEDEMKAQMDCFYLKALDGFVMVLTDDGDMVYISDNVNKYMGLTQFELTGHSVFDFTHPCDHEEMREMLTHRNGPVRKGKELNTQRSFFLRMKCTLTSRGRTMNIKSATWKVLHCTGHIHVYDTNSNQPQCGYKKPPMTCLVLICEPIPHPSNIEIPLDSKTFLSRHSLDMKFSYCDERITELMGYEPEELLGRSIYEYYHALDSDHLTKTHHDMFTKGQVTTGQYRMLAKRGGYVWVETQATVIYNTKNSQPQCIVCVNYVVSGIIQHDLIFSLQQTESVLKPVESSDMKMTQLFTKVESEDTSCLFDKLKKEPDALTLLAPAAGDTIISLDFGSDDTETEDQQLEDVPLYNDVMFPSSNEKLNINLAMSPLPSSETPKPLRSSADPALNQEVALKLESSPESLGLSFTMPQIQDQPASPSDGSTRQSSPERLLQENVNTPNFSQPNSPSEYCFDVDSDMVNVFKLELVEKLFAEDTEAKNPFSTQDTDLDLEMLAPYIPMDDDFQLRSFDQLSPLESNSPSPPSMSTVTGFQQTQLQKPTITATATTTATTDESKTETKDNKEDIKILIASPSSTQVPQETTTAKASAYSGTHSRTASPDRAGKRVIEQTDKAHPRSLKLSATLNQRNTVPEEELNPKTIASQNAQRKRKMEHDGSLFQAAGIGTLLQQPGDCAPTMSLSWKRVKGFISSEQNGTEQKTIILIPSDLACRLLGQSMDESGLPQLTSYDCEVNAPIQGSRNLLQGEELLRALDQVN

SEQ ID NO: 4 - protein, 823 aa, Rattus norvegicus
MEGAGGENEKKNRMSSERRKEKSRDAARSRRSKESEVFYELAHQLPLPHNVSSHLDKASVMRLTISYLRVRKLLGAGDLDIEDEMKAQMNCFYLKALDGFVMVLTDDGDMIYISDNVNKYMGLTQFELTGHSVFDFTHPCDHEEMREMLTHRNGPVRKGKEQNTQRSFFLRMKCTLTSRGRTMNIKSATWKVLHCTGHIHVYDTSSNQPQCGYKKPPMTCLVLICEPIPHPSNIEIPLDSKTFLSRHSLDMKFSYCDERITELMGYEPEELLGRSIYEYYHALDSDHLTKTHHDMFTKGQVTTGQYRMLAKRGGYVWVETQATVIYNTKNSQPQCIVCVNYVVSGIIQHDLIFSLQQTESVLKPVESSDMKMTQLFTKVESEDTSCLFDKLKKEPDALTLLAPAAGDTIISLDFGSDDTETEDQQLEDVPLYNDVMFPSSNEKLNINLAMSPLPASETPKPLRSSADPALNQEVALKLESSPESLGLSFTMPQIQDQPASPSDGSTRQSSPEPNSPSEYCFDVDSDMVNVFKLELVEKLFAEDTEAKNPFSAQDTDLDLEMLAPYIPMDDDFQLRSFDQLSPLESNSPSPPSVSTVTGFQQTQLQKPTITVTATATATTDESKAVTKDNIEDIKILIASPPSTQVPQEMTTAKASAYSGTHSRTASPDRAGKRVIEKTDKAHPRSLNLSVTLNQRNTVPEEELNPKTIALQNAQRKRKMEHDGSLFQAAGIGTLLQQPGDRAPTMSLSWKRVKGYISSEQDGMEQKTIFLIPSDLACRLLGQSMDESGLPQLTSYDCEVNAPIQGSRNLLQGEELLRALDQVN

SEQ ID NO: 5 - DNA, 3958 bp, Homo sapiens
gtgctgcctcgtctgaggggacaggaggatcaccctcttcgtcgcttcggccagtgtgtcgggctgggccctgacaagccacctgaggagaggctcggagccgggcccggaccccggcgattgccgcccgcttctctctagtctcacgaggggtttcccgcctcgcacccccacctctggacttgcctttccttctcttctccgcgtgtggagggagccagcgcttaggccggagcgagcctgggggccgcccgccgtgaagacatcgcggggaccgattcaccatggagggcgccggcggcgcgaacgacaagaaaaagataagttctgaacgtcgaaaagaaaagtctcgagatgcagccagatctcggcgaagtaaagaatctgaagttttttatgagcttgctcatcagttgccacttccacataatgtgagttcgcatcttgataaggcctctgtgatgaggcttaccatcagctatttgcgtgtgaggaaacttctggatgctggtgatttggatattgaagatgacatgaaagcacagatgaattgcttttatttgaaagccttggatggttttgttatggttctcacagatgatggtgacatgatttacatttctgataatgtgaacaaatacatgggattaactcagtttgaactaactggacacagtgtgtttgattttactcatccatgtgaccatgaggaaatgagagaaatgcttacacacagaaatggccttgtgaaaaagggtaaagaacaaaacacacagcgaagcttttttctcagaatgaagtgtaccctaactagccgaggaagaactatgaacataaagtctgcaacatggaaggtattgcactgcacaggccacattcacgtatatgataccaacagtaaccaacctcagtgtgggtataagaaaccacctatgacctgcttggtgctgatttgtgaacccattcctcacccatcaaatattgaaattcctttagatagcaagactttcctcagtcgacacagcctggatatgaaattttcttattgtgatgaaagaattaccgaattgatgggatatgagccagaagaacttttaggccgctcaatttatgaatattatcatgctttggactctgatcatctgaccaaaactcatcatgatatgtttactaaaggacaagtcaccacaggacagtacaggatgcttgccaaaagaggtggatatgtctgggttgaaactcaagcaactgtcatatataacaccaagaattctcaaccacagtgcattgtatgtgtgaattacgttgtgagtggtattattcagcacgacttgattttctcccttcaacaaacagaatgtgtccttaaaccggttgaatcttcagatatgaaaatgactcagctattcaccaaagttgaatcagaagatacaagtagcctctttgacaaacttaagaaggaacctgatgctttaactttgctggccccagccgctggagacacaatcatatctttagattttggcagcaacgacacagaaactgatgaccagcaacttgaggaagtaccattatataatgatgtaatgctcccctcacccaacgaaaaattacagaatataaatttggcaatgtctccattacccaccgctgaaacgccaaagccacttcgaagtagtgctgaccctgcactcaatcaagaagttgcattaaaattagaaccaaatccagagtcactggaactttcttttaccatgccccagattcaggatcagacacctagtccttccgatggaagcactagacaaagttcacctgagcctaatagtcccagtgaatattgtttttatgtggatagtgatatggtcaatgaattcaagttggaattggtagaaaaactttttgctgaagacacagaagcaaagaacccattttctactcaggacacagatttagacttggagatgttagctccctatatcccaatggatgatgacttccagttacgttccttcgatcagttgtcaccattagaaagcagttccgcaagccctgaaagcgcaagtcctcaaagcacagttacagtattccagcagactcaaatacaagaacctactgctaatgccaccactaccactgccaccactgatgaattaaaaacagtgacaaaagaccgtatggaagacattaaaatattgattgcatctccatctcctacccacatacataaagaaactactagtgccacatcatcaccatatagagatactcaaagtcggacagcctcaccaaacagagcaggaaaaggagtcatagaacagacagaaaaatctcatccaagaagccctaacgtgttatctgtcgctttgagtcaaagaactacagttcctgaggaagaactaaatccaaagatactagctttgcagaatgctcagagaaagcgaaaaatggaacatgatggttcactttttcaagcagtaggaattggaacattattacagcagccagacgatcatgcagctactacatcactttcttggaaacgtgtaaaaggatgcaaatctagtgaacagaatggaatggagcaaaagacaattattttaataccctctgatttagcatgtagactgctggggcaatcaatggatgaaagtggattaccacagctgaccagttatgattgtgaagttaatgctcctatacaaggcagcagaaacctactgcagggtgaagaattactcagagctttggatcaagttaactgagctttttcttaatttcattcctttttttggacactggtggctcactacctaaagcagtctatttatattttctacatctaattttagaagcctggctacaatactgcacaaacttggttagttcaatttttgatcccctttctacttaatttacattaatgctcttttttagtatgttctttaatgctggatcacagacagctcattttctcagttttttggtatttaaaccattgcattgcagtagcatcattttaaaaaatgcacctttttatttatttatttttggctagggagtttatccctttttcgaattatttttaagaagatgccaatataatttttgtaagaaggcagtaacctttcatcatgatcataggcagttgaaaaatttttacaccttttttttcacattttacataaataataatgctttgccagcagtacgtggtagccacaattgcacaatatattttcttaaaaaataccagcagttactcatggaatatattctgcgtttataaaactagtttttaagaagaaattttttttggcctatgaaattgttaaacctggaacatgacattgttaatcatataataatgattcttaaatgctgtatggtttattatttaaatgggtaaagccatttacataatatagaaagatatgcatatatctagaaggtatgtggcatttatttggataaaattctcaattcagagaaatcatctgatgtttctatagtcactttgccagctcaaaagaaaacaataccctatgtagttgtggaagtttatgctaatattgtgtaactgatattaaacctaaatgttctgcctaccctgttggtataaagatattttgagcagactgtaaacaagaaaaaaaaaatcatgcattcttagcaaaattgcctagtatgttaatttgctcaaaatacaatgtttgattttatgcactttgtcgctattaacatcctttttttcatgtagatttcaataattgagtaattttagaagcattattttaggaatatatagttgtcacagtaaatatcttgttttttctatgtacattgtacaaatttttcattccttttgctctttgtggttggatctaacactaactgtattgttttgttacatcaaataaacatcttctgtggaccaggaaaaaaaaaaaaaaaaaaa

SEQ ID NO: 6 - DNA, 3812 bp, Homo sapiens
gtgctgcctcgtctgaggggacaggaggatcaccctcttcgtcgcttcggccagtgtgtcgggctgggccctgacaagccacctgaggagaggctcggagccgggcccggaccccggcgattgccgcccgcttctctctagtctcacgaggggtttcccgcctcgcacccccacctctggacttgcctttccttctcttctccgcgtgtggagggagccagcgcttaggccggagcgagcctgggggccgcccgccgtgaagacatcgcggggaccgattcaccatggagggcgccggcggcgcgaacgacaagaaaaagataagttctgaacgtcgaaaagaaaagtctcgagatgcagccagatctcggcgaagtaaagaatctgaagttttttatgagcttgctcatcagttgccacttccacataatgtgagttcgcatcttgataaggcctctgtgatgaggcttaccatcagctatttgcgtgtgaggaaacttctggatgctggtgatttggatattgaagatgacatgaaagcacagatgaattgcttttatttgaaagccttggatggttttgttatggttctcacagatgatggtgacatgatttacatttctgataatgtgaacaaatacatgggattaactcagtttgaactaactggacacagtgtgtttgattttactcatccatgtgaccatgaggaaatgagagaaatgcttacacacagaaatggccttgtgaaaaagggtaaagaacaaaacacacagcgaagcttttttctcagaatgaagtgtaccctaactagccgaggaagaactatgaacataaagtctgcaacatggaaggtattgcactgcacaggccacattcacgtatatgataccaacagtaaccaacctcagtgtgggtataagaaaccacctatgacctgcttggtgctgatttgtgaacccattcctcacccatcaaatattgaaattcctttagatagcaagactttcctcagtcgacacagcctggatatgaaattttcttattgtgatgaaagaattaccgaattgatgggatatgagccagaagaacttttaggccgctcaatttatgaatattatcatgctttggactctgatcatctgaccaaaactcatcatgatatgtttactaaaggacaagtcaccacaggacagtacaggatgcttgccaaaagaggtggatatgtctgggttgaaactcaagcaactgtcatatataacaccaagaattctcaaccacagtgcattgtatgtgtgaattacgttgtgagtggtattattcagcacgacttgattttctcccttcaacaaacagaatgtgtccttaaaccggttgaatcttcagatatgaaaatgactcagctattcaccaaagttgaatcagaagatacaagtagcctctttgacaaacttaagaaggaacctgatgctttaactttgctggccccagccgctggagacacaatcatatctttagattttggcagcaacgacacagaaactgatgaccagcaacttgaggaagtaccattatataatgatgtaatgctcccctcacccaacgaaaaattacagaatataaatttggcaatgtctccattacccaccgctgaaacgccaaagccacttcgaagtagtgctgaccctgcactcaatcaagaagttgcattaaaattagaaccaaatccagagtcactggaactttcttttaccatgccccagattcaggatcagacacctagtccttccgatggaagcactagacaaagttcacctgagcctaatagtcccagtgaatattgtttttatgtggatagtgatatggtcaatgaattcaagttggaattggtagaaaaactttttgctgaagacacagaagcaaagaacccattttctactcaggacacagatttagacttggagatgttagctccctatatcccaatggatgatgacttccagttacgttccttcgatcagttgtcaccattagaaagcagttccgcaagccctgaaagcgcaagtcctcaaagcacagttacagtattccagcagactcaaatacaagaacctactgctaatgccaccactaccactgccaccactgatgaattaaaaacagtgacaaaagaccgtatggaagacattaaaatattgattgcatctccatctcctacccacatacataaagaaactactagtgccacatcatcaccatatagagatactcaaagtcggacagcctcaccaaacagagcaggaaaaggagtcatagaacagacagaaaaatctcatccaagaagccctaacgtgttatctgtcgctttgagtcaaagaactacagttcctgaggaagaactaaatccaaagatactagctttgcagaatgctcagagaaagcgaaaaatggaacatgatggttcactttttcaagcagtaggaattatttagcatgtagactgctggggcaatcaatggatgaaagtggattaccacagctgaccagttatgattgtgaagttaatgctcctatacaaggcagcagaaacctactgcagggtgaagaattactcagagctttggatcaagttaactgagctttttcttaatttcattcctttttttggacactggtggctcactacctaaagcagtctatttatattttctacatctaattttagaagcctggctacaatactgcacaaacttggttagttcaatttttgatcccctttctacttaatttacattaatgctcttttttagtatgttctttaatgctggatcacagacagctcattttctcagttttttggtatttaaaccattgcattgcagtagcatcattttaaaaaatgcacctttttatttatttatttttggctagggagtttatccctttttcgaattatttttaagaagatgccaatataatttttgtaagaaggcagtaacctttcatcatgatcataggcagttgaaaaatttttacaccttttttttcacattttacataaataataatgctttgccagcagtacgtggtagccacaattgcacaatatattttcttaaaaaataccagcagttactcatggaatatattctgcgtttataaaactagtttttaagaagaaattttttttggcctatgaaattgttaaacctggaacatgacattgttaatcatataataatgattcttaaatgctgtatggtttattatttaaatgggtaaagccatttacataatatagaaagatatgcatatatctagaaggtatgtggcatttatttggataaaattctcaattcagagaaatcatctgatgtttctatagtcactttgccagctcaaaagaaaacaataccctatgtagttgtggaagtttatgctaatattgtgtaactgatattaaacctaaatgttctgcctaccctgttggtataaagatattttgagcagactgtaaacaagaaaaaaaaaatcatgcattcttagcaaaattgcctagtatgttaatttgctcaaaatacaatgtttgattttatgcactttgtcgctattaacatcctttttttcatgtagatttcaataattgagtaattttagaagcattattttaggaatatatagttgtcacagtaaatatcttgttttttctatgtacattgtacaaatttttcattccttttgctctttgtggttggatctaacactaactgtattgttttgttacatcaaataaacatcttctgtggaccagg

SEQ ID NO: 7 - DNA, 3973 bp, Mus musculus
cgcgaggactgtcctcgccgccgtcgcgggcagtgtctagccaggccttgacaagctagccggaggagcgcctaggaacccgagccggagctcagcgagcgcagcctgcacgcccgcctcgcgtcccgggggggtcccgcctcccaccccgcctctggacttgtctctttccccgcgcgcgcggacagagccggcgtttaggcccgagcgagcccgggggccgccggccgggaagacaacgcgggcaccgattcgccatggagggcgccggcggcgagaacgagaagaaaaagatgagttctgaacgtcgaaaagaaaagtctagagatgcagcaagatctcggcgaagcaaagagtctgaagttttttatgagcttgctcatcagttgccacttccccacaatgtgagctcacatcttgataaagcttctgttatgaggctcaccatcagttatttacgtgtgagaaaacttctggatgccggtggtctagacagtgaagatgagatgaaggcacagatggactgtttttatctgaaagccctagatggctttgtgatggtgctaacagatgacggcgacatggtttacatttctgataacgtgaacaaatacatggggttaactcagtttgaactaactggacacagtgtgtttgattttactcatccatgtgaccatgaggaaatgagagaaatgcttacacacagaaatggcccagtgagaaaagggaaagaactaaacacacagcggagcttttttctcagaatgaagtgcaccctaacaagccgggggaggacgatgaacatcaagtcagcaacgtggaaggtgcttcactgcacgggccatattcatgtctatgataccaacagtaaccaacctcagtgtgggtacaagaaaccacccatgacgtgcttggtgctgatttgtgaacccattcctcatccgtcaaatattgaaattcctttagatagcaagacatttctcagtcgacacagcctcgatatgaaattttcttactgtgatgaaagaattactgagttgatgggttatgagccggaagaacttttgggccgctcaatttatgaatattatcatgctttggattctgatcatctgaccaaaactcaccatgatatgtttactaaaggacaagtcaccacaggacagtacaggatgcttgccaaaagaggtggatatgtctgggttgaaactcaagcaactgtcatatataatacgaagaactcccagccacagtgcattgtgtgtgtgaattatgttgtaagtggtattattcagcacgacttgattttctcccttcaacaaacagaatctgtgctcaaaccagttgaatcttcagatatgaagatgactcagctgttcaccaaagttgaatcagaggatacaagctgcctttttgataagcttaagaaggagcctgatgctctcactctgctggctccagctgccggcgacaccatcatctctctggattttggcagcgatgacacagaaactgaagatcaacaacttgaagatgttccattatataatgatgtaatgtttccctcttctaatgaaaaattaaatataaacctggcaatgtctcctttaccttcatcggaaactccaaagccacttcgaagtagtgctgatcctgcactgaatcaagaggttgcattaaaattagaatcaagtccagagtcactgggactttcttttaccatgccccagattcaagatcagccagcaagtccttctgatggaagcactagacaaagttcacctgagagacttcttcaggaaaacgtaaacactcctaacttttcccagcctaacagtcccagtgaatattgctttgatgtggatagcgatatggtcaatgtattcaagttggaactggtggaaaaactgtttgctgaagacacagaggcaaagaatccattttcaactcaggacactgatttagatttggagatgctggctccctatatcccaatggatgatgatttccagttacgttcctttgatcagttgtcaccattagagagcaattctccaagccctccaagtatgagcacagttactgggttccagcagacccagttacagaaacctaccatcactgccactgccaccacaactgccaccactgatgaatcaaaaacagagacgaaggacaataaagaagatattaaaatactgattgcatctccatcttctacccaagtacctcaagaaacgaccactgctaaggcatcagcatacagtggcactcacagtcggacagcctcaccagacagagcaggaaagagagtcatagaacagacagacaaagctcatccaaggagccttaagctgtctgccactttgaatcaaagaaatactgttcctgaggaagaattaaacccaaagacaatagcttcgcagaatgctcagaggaagcgaaaaatggaacatgatggctccctttttcaagcagcaggaattggaacattattgcagcaaccaggtgactgtgcacctactatgtcactttcctggaaacgagtgaaaggattcatatctagtgaacagaatggaacggagcaaaagactattattttaataccctccgatttagcatgcagactgctggggcagtcaatggatgagagtggattaccacagctgaccagttacgattgtgaagttaatgctcccatacaaggcagcagaaacctactgcagggtgaagaattactcagagctttggatcaagttaactgagcgtttcctaatctcattccttttgattgttaatgtttttgttcagttgttgttgtttgttgggtttttgtttctgttggttatttttggacactggtggctcagcagtctatttatattttctatatctaattttagaagcctggctacaatactgcacaaactcagatagtttagttttcatcccctttctacttaattttcattaatgctctttttaatatgttcttttaatgccagatcacagcacattcacagctcctcagcatttcaccattgcattgctgtagtgtcatttaaaatgcacctttttatttatttatttttggtgagggagtttgtcccttattgaattatttttaatgaaatgccaatataattttttaagaaagcagtaaattctcatcatgatcataggcagttgaaaactttttactcatttttttcatgttttacatgaaaataatgctttgtcagcagtacatggtagccacaattgcacaatatattttctttaaaaaaccagcagttactcatgcaatatattctgcatttataaaactagtttttaagaaattttttttggcctatggaattgttaagcctggatcatgaagcgttgatcttataatgattcttaaactgtatggtttctttatatgggtaaagccatttacatgatataaagaaatatgcttatatctggaaggtatgtggcatttatttggataaaattctcaattcagagaagttatctggtgtttcttgactttaccaactcaaaacagtccctctgtagttgtggaagcttatgctaatattgtgtaattgattatgaaacataaatgttctgcccaccctgttggtataaagacattttgagcatactgtaaacaaacaaacaaaaaatcatgctttgttagtaaaattgcctagtatgttgatttgttgaaaatatgatgtttggttttatgcactttgtcgctattaacatccttttttcatatagatttcaataagtgagtaattttagaagcattattttaggaatatagagttgtcatagtaaacatcttgttttttctatgtacactgtataaatttttcgttcccttgctctttgtggttgggtctaacactaactgtactgttttgttatatcaaataaacatcttctgtggaccaggaaaaaaaaaaaaaaaaaa

SEQ ID NO: 8 - DNA, 3718 bp, Rattus norvegicus
gacaccgcgggcaccgattcgccatggagggcgccggcggcgagaacgagaagaaaaataggatgagttccgaacgtcgaaaagaaaagtctagggatgcagcacgatctcggcgaagcaaagagtctgaagttttttatgagcttgctcatcagttgccacttccccacaacgtgagctcccatcttgataaagcttctgttatgaggctcaccatcagttacttacgtgtgaggaaacttctaggtgctggtgatcttgacattgaagatgaaatgaaagcacagatgaactgcttttatctgaaagccctggatggctttgttatggtgctaacagatgatggtgacatgatttacatttctgataacgtgaacaaatacatggggttgactcagtttgaactaactggacacagtgtgtttgattttacccatccatgtgaccatgaggaaatgagagaaatgcttacacacagaaatggcccagtgagaaaggggaaagaacaaaacacgcagcgaagcttttttctcagaatgaaatgtaccctaacaagccgggggaggacgatgaacatcaagtcagcaacgtggaaggtgctgcactgcacaggccacattcatgtgtatgataccagcagtaaccagccgcagtgtggctacaagaaaccgcctatgacgtgcttggtgctgatttgtgaacccattcctcatccatcaaacattgaaattcctttagacagcaagacatttctcagtcgacacagcctcgatatgaaattttcttactgtgatgaaaggattactgagttgatgggttatgagccagaagaacttttgggccgttcaatttatgaatattatcatgctttggactctgatcatctgaccaaaactcatcatgacatgtttactaaaggacaagtcaccacaggacagtacaggatgcttgcaaaaagaggtggatatgtctgggttgagactcaagcaactgttatatataatacgaagaactctcagccacagtgcattgtgtgtgtgaattatgttgtaagtggtattattcagcacgacttgattttctcccttcaacaaacagaatctgtcctcaaaccagttgaatcttcagatatgaaaatgacccagctgttcactaaagtggaatctgaggacacgagctgcctcttcgacaagcttaagaaagagcccgatgccctgactctgctagctccagcggctggggacacgatcatatcactggacttcggcagcgatgacacggaaactgaagaccaacaacttgaagatgtcccgttgtacaatgatgtaatgttcccctcttctaatgagaaattaaatataaatctggcaatgtctccattacctgcctctgaaactccaaagccacttcgaagtagtgctgatcctgcactgaatcaagaggttgcattgaagttagagtcaagcccagagtcactgggactttcttttaccatgccccagattcaagatcagccagcaagtccttctgatggaagcactagacaaagctcacctgagcctaacagtcccagtgagtactgctttgatgtggacagcgatatggtcaatgtattcaagttggaactggtggaaaaactgtttgctgaagacacagaagcgaagaatccattttcagctcaggacactgatttagacttggaaatgctggctccctatatcccaatggatgatgatttccagttacgttcctttgatcagttgtcaccattagagagcaattctccaagccctccgagtgtgagcacagttacaggattccagcagacccagttacagaaacctaccatcactgtcactgccaccgcaactgccaccactgatgaatcaaaagcagtgacgaaggacaatatagaagacattaaaatactgattgcatctccaccttctacccaagtacctcaagaaatgaccactgctaaggcatcagcatacagtggtactcacagtcggacagcctcaccagacagagcaggaaagagagtcatagaaaaaacagacaaagctcatccaaggagccttaacctatctgtcactttgaatcaaagaaatactgttcctgaagaagaattaaacccaaagacaatagctttgcagaatgctcagaggaagcgaaaaatggaacatgatggctccctttttcaagcagcaggaattggaacgttactgcagcaaccaggtgaccgtgcccctactatgtcgctttcttggaaacgagtgaaaggatacatatctagtgaacaggatggaatggagcagaagacaatttttttaataccctctgatttagcatgtagactgctggggcagtcaatggatgagagtggattaccacagctgaccagttacgattgtgaagttaatgctcccatacaaggcagcagaaacctactgcagggtgaagaattactcagagctttggatcaagttaactgagcttttcctaatctcattcctttgattgttaatttttgtgttcagttgttgttgttgtctgtggggtttcgtttctgttggttgttttggacactggtggctcagcagtctatttatattttctatatctcatttagaggcctggctacagtactgcaccaactcagatagtttagtttgggccccttcctccttcattttcactgatgctctttttaccatgtccttcgaatgccagatcacagcacattcacagctccccagcatttcaccaatgcattgctgtagtgtcgtttaaaatgcacctttttatttatttatttttggtgagggagtttgtcccttattgaattatttttaatgaaatgccaatataattttttaagaaggcagtaaatcttcatcatgatgataggcagttgaaaattttttactcatttttttcatgttttacatgaaaataatgctttgccagcagtacatggtagccacaattgcacaatatattttcttaaaaataccagcagttactcatgcatatattctgcatttataaaactagtttttaagaagaaactttttttggcctatggaattgttaagcctggatcatgatgctgttgatcttataatgattcttaaactgtatggtttctttatatgggtaaagccatttacatgatatagagagatatgcttatatctggaaggtatatggcatttatttggataaaattctcaattgagaagttatctggtgtttctttactttaccggctcaaaagaaaacagtccctatgtagttgtggaagcttatgctaatattgtgtaattgatattaaacattaaatgttctgcctatcctgttggtataaagacattttgagcatactgtaaacaaaaaaatcatgcattgttagtaaaattgcctagtatgttaatttgttgaaaatacgatgtttggttttatgcactttgtcgctattaacatcctttttttcatatagatttcaataattgagtaattttagaagcattattttagaaatatagagttgtcatagtaaacatcttgtttttttttctttttttctatgtacattgtataaatttttcattcccttgctctttgtagttgggtctaacactaactgtactgttttgttatatcaaataaacatcttctgtggaccaggaaaaaaaaaaaaaaaaaaaaaaaaaaa

SEQ ID NO: 9 - protein, 213 aa, Homo sapiens
MPRRAENWDEAEVGAEEAGVEEYGPEEDGGEESGAEESGPEESGPEELGAEEEMEAGRPRPVLRSVNSREPSQVIFCNRSPRVVLPVWLNFDGEPQPYPTLPPGTGRRIHSYRGHLWLFRDAGTHDGLLVNQTELFVPSLNVDGQPIFANITLPVYTLKERCLQVVRSLVKPENYRRLDIVRSLYEDLEDHPNVQKDLERLTQERIAHQRMGD

SEQ ID NO: 10 - protein, 172 aa, Homo sapiens
MPRRAENWDEAEVGAEEAGVEEYGPEEDGGEESGAEESGPEESGPEELGAEEEMEAGRPRPVLRSVNSREPSQVIFCNRSPRVVLPVWLNFDGEPQPYPTLPPGTGRRIHSYRVYTLKERCLQVVRSLVKPENYRRLDIVRSLYEDLEDHPNVQKDLERLTQERIAHQRMGD

SEQ ID NO: 11 - protein, 181 aa, Mus musculus
MPRKAASPEEAAGEPGPEEMEAGRPRPVLRSVNSREPSQVIFCNRSPRVVLPLWLNFDGEPQPYPILPPGTGRRIHSYRGHLWLFRDAGTHDGLLVNQTELFVPSLNVDGQPIFANITLPVYTLKERCLQVVRSLVKPENYRRLDIVRSLYEDLEDYPSVRKDIQRLSQEHLESQHLEEEP

SEQ ID NO: 12 - protein, 185 aa, Rattus norvegicus
MPRKAASPEEAERMPGSEEIEAGRPRPVLRSVNSREPSQVIFCNRSPRVVLPLWLNFDGEPQPYPTLPPGTGRRIHSYRGHLWLFRDAGTHDGLLVNQTELFVPSLNVDGQPIFANITLPVYTLKERCLQVVRSLVKPENYRRLDIVRSLYEDLEDHPNVRKDIQRLTQEHLENQALGEEPEGVH

SEQ ID NO: 13 - DNA, 2968 bp, Homo sapiens
cctcgcctccgttacaacggcctacggtgctggaggatccttctgcgcacgcgcacagcctccggccggctatttccgcgagcgcgttccatcctctaccgagcgcgcgcgaagactacggaggtcgactcgggagcgcgcacgcagctccgccccgcgtccgacccgcggatcccgcggcgtccggcccgggtggtctggatcgcggagggaatgccccggagggcggagaactgggacgaggccgaggtaggcgcggaggaggcaggcgtcgaagagtacggccctgaagaagacggcggggaggagtcgggcgccgaggagtccggcccggaagagtccggcccggaggaactgggcgccgaggaggagatggaggccgggcggccgcggcccgtgctgcgctcggtgaactcgcgcgagccctcccaggtcatcttctgcaatcgcagtccgcgcgtcgtgctgcccgtatggctcaacttcgacggcgagccgcagccctacccaacgctgccgcctggcacgggccgccgcatccacagctaccgaggtcacctttggctcttcagagatgcagggacacacgatgggcttctggttaaccaaactgaattatttgtgccatctctcaatgttgacggacagcctatttttgccaatatcacactgccagtgtatactctgaaagagcgatgcctccaggttgtccggagcctagtcaagcctgagaattacaggagactggacatcgtcaggtcgctctacgaagatctggaagaccacccaaatgtgcagaaagacctggagcggctgacacaggagcgcattgcacatcaacggatgggagattgaagatttctgttgaaacttacactgtttcatctcagcttttgatggtactgatgagtcttgatctagatacaggactggttccttccttagtttcaaagtgtctcattctcagagtaaaataggcaccattgcttaaaagaaagttaactgacttcactaggcattgtgatgtttaggggcaaacatcacaaaatgtaatttaatgcctgcccattagagaagtatttatcaggagaaggtggtggcatttttgcttcctagtaagtcaggacagcttgtatgtaaggaggtttgtataagtaattcagtgggaattgcagcatatcgtttaattttaagaaggcattggcatctgcttttaatggatgtataatacatccattctacatccgtagcggttggtgacttgtctgcctcctgctttgggaagactgaggcatccgtgaggcagggacaagtctttctcctctttgagaccccagtgcctgcacatcatgagccttcagtcagggtttgtcagaggaacaaaccaggggacactttgttagaaagtgcttagaggttctgcctctatttttgttggggggtgggagaggggaccttaaaatgtgtacagtgaacaaatgtcttaaagggaatcatttttgtaggaagcattttttataattttctaagtcgtgcactttctcggtccactcttgttgaagtgctgttttattactgtttctaaactaggattgacattctacagttgtgataatagcatttttgtaacttgccatccgcacagaaaatacgagaaaatctgcatgtttgattatagtattaatggacaaataagtttttgctaaatgtgagtatttctgttcctttttgtaaatatgtgacattcctgattgatttgggtttttttgttgttgttgttttgttttgttttgtttttttgagatggagtctcactcttgtcacccaggctggagtgcagtggcgccatctcggctcactgcaacctctgcctcctgggttcacgtaatcctcctgagtagctgggattacaggcgcctgccaccacgctggccaatttttgtacttttagtagagacagtgtttcgccatgttggccaggctggtttcaaactcctgacctcaggtgatccgcccacctcagcctcccaaaatggtgggattacaggtgtgtgggccaccgtgcctggctgattcagcattttttatcaggcaggaccaggtggcacttccacctccagcctctggtcctaccaatggattcatggagtagcctggactgtttcatagttttctaaatgtacaaattcttataggctagacttagattcattaactcaaattcaatgcttctatcagactcagttttttgtaactaatagatttttttttccacttttgttctactccttccctaatagctttttaaaaaaatctccccagtagagaaacatttggaaaagacagaaaactaaaaaggaagaaaaaagatccctattagatacacttcttaaatacaatcacattaacattttgagctatttccttccagcctttttagggcagattttggttggtttttacatagttgagattgtactgttcatacagttttataccctttttcatttaactttataacttaaatattgctctatgttagtataagcttttcacaaacattagtatagtctcccttttataattaatgtttgtgggtatttcttggcatgcatctttaattccttatcctagcctttgggcacaattcctgtgctcaaaaatgagagtgacggctggcatggtggctcccgcctgtaatcccagtactttgggaagccaaggtaagaggattgcttgagcccagaacttcaagatgagcctgggctcatagtgagaacccatctatacaaaaaatttttaaaaattagcatggcggcacacatctgtaatcctagctacttggcaggctgaggtgagaagatcattggagtttaggaattggaggctgcagtgagccatgagtatgccactgcactccagcctgggggacagagcaagaccctgcctcaaaaaaaaaaaaaaaaaa

SEQ ID NO: 14 - DNA, 2845 bp, Homo sapiens
cctcgcctccgttacaacggcctacggtgctggaggatccttctgcgcacgcgcacagcctccggccggctatttccgcgagcgcgttccatcctctaccgagcgcgcgcgaagactacggaggtcgactcgggagcgcgcacgcagctccgccccgcgtccgacccgcggatcccgcggcgtccggcccgggtggtctggatcgcggagggaatgccccggagggcggagaactgggacgaggccgaggtaggcgcggaggaggcaggcgtcgaagagtacggccctgaagaagacggcggggaggagtcgggcgccgaggagtccggcccggaagagtccggcccggaggaactgggcgccgaggaggagatggaggccgggcggccgcggcccgtgctgcgctcggtgaactcgcgcgagccctcccaggtcatcttctgcaatcgcagtccgcgcgtcgtgctgcccgtatggctcaacttcgacggcgagccgcagccctacccaacgctgccgcctggcacgggccgccgcatccacagctaccgagtgtatactctgaaagagcgatgcctccaggttgtccggagcctagtcaagcctgagaattacaggagactggacatcgtcaggtcgctctacgaagatctggaagaccacccaaatgtgcagaaagacctggagcggctgacacaggagcgcattgcacatcaacggatgggagattgaagatttctgttgaaacttacactgtttcatctcagcttttgatggtactgatgagtcttgatctagatacaggactggttccttccttagtttcaaagtgtctcattctcagagtaaaataggcaccattgcttaaaagaaagttaactgacttcactaggcattgtgatgtttaggggcaaacatcacaaaatgtaatttaatgcctgcccattagagaagtatttatcaggagaaggtggtggcatttttgcttcctagtaagtcaggacagcttgtatgtaaggaggtttgtataagtaattcagtgggaattgcagcatatcgtttaattttaagaaggcattggcatctgcttttaatggatgtataatacatccattctacatccgtagcggttggtgacttgtctgcctcctgctttgggaagactgaggcatccgtgaggcagggacaagtctttctcctctttgagaccccagtgcctgcacatcatgagccttcagtcagggtttgtcagaggaacaaaccaggggacactttgttagaaagtgcttagaggttctgcctctatttttgttggggggtgggagaggggaccttaaaatgtgtacagtgaacaaatgtcttaaagggaatcatttttgtaggaagcattttttataattttctaagtcgtgcactttctcggtccactcttgttgaagtgctgttttattactgtttctaaactaggattgacattctacagttgtgataatagcatttttgtaacttgccatccgcacagaaaatacgagaaaatctgcatgtttgattatagtattaatggacaaataagtttttgctaaatgtgagtatttctgttcctttttgtaaatatgtgacattcctgattgatttgggtttttttgttgttgttgttttgttttgttttgtttttttgagatggagtctcactcttgtcacccaggctggagtgcagtggcgccatctcggctcactgcaacctctgcctcctgggttcacgtaatcctcctgagtagctgggattacaggcgcctgccaccacgctggccaatttttgtacttttagtagagacagtgtttcgccatgttggccaggctggtttcaaactcctgacctcaggtgatccgcccacctcagcctcccaaaatggtgggattacaggtgtgtgggccaccgtgcctggctgattcagcattttttatcaggcaggaccaggtggcacttccacctccagcctctggtcctaccaatggattcatggagtagcctggactgtttcatagttttctaaatgtacaaattcttataggctagacttagattcattaactcaaattcaatgcttctatcagactcagttttttgtaactaatagatttttttttccacttttgttctactccttccctaatagctttttaaaaaaatctccccagtagagaaacatttggaaaagacagaaaactaaaaaggaagaaaaaagatccctattagatacacttcttaaatacaatcacattaacattttgagctatttccttccagcctttttagggcagattttggttggtttttacatagttgagattgtactgttcatacagttttataccctttttcatttaactttataacttaaatattgctctatgttagtataagcttttcacaaacattagtatagtctcccttttataattaatgtttgtgggtatttcttggcatgcatctttaattccttatcctagcctttgggcacaattcctgtgctcaaaaatgagagtgacggctggcatggtggctcccgcctgtaatcccagtactttgggaagccaaggtaagaggattgcttgagcccagaacttcaagatgagcctgggctcatagtgagaacccatctatacaaaaaatttttaaaaattagcatggcggcacacatctgtaatcctagctacttggcaggctgaggtgagaagatcattggagtttaggaattggaggctgcagtgagccatgagtatgccactgcactccagcctgggggacagagcaagaccctgcctcaaaaaaaaaaaaaaaaaa

SEQ ID NO: 15 - DNA, 2730 bp, Mus musculus
cgccttcaagttgcccggagcgcgtctgggaccggcagacctgtcccggagcgcgtagatccgcacgcacgcacgcgccgcccacgtccagcttgcgaatccgagggacccgttccaataatgccccggaaggcagccagtccagaggaggcggcgggggagcccggtcctgaggagatggaggctgggcggccgcggccggtgctgcgctcggtgaactcgcgcgagccctctcaggtcatcttctgcaaccgcagcccgcgcgtcgtgctgcctttgtggctcaacttcgacggcgagcctcagccctacccgatcttaccaccgggcaccggccgccgcatccacagctaccgaggtcatctttggctcttcagggatgcggggacccatgatggacttctggttaaccaaacggagctgtttgtgccatccctcaatgtcgatggacagcctatttttgccaacatcacattgccagtgtataccctgaaagagcggtgccttcaggttgtgcggagcctggtcaagcctgagaactacaggagactggacatcgtcaggtcactctatgaggatttggaggactacccaagtgtgcggaaggacatacagcgactgagccaagagcaccttgagagtcagcacctggaagaggagccttgaaggagtccatggagattaagtgttcctgagtttcagccttgatggtccgagattgatctacacataggacaggtcactttctttcagttttaaaatggttcattcttggagtaaaactatccatcacgtaaaagaaagttaactaacatccctgggctttgtagtgtttaagaataaacatgcaaagtgccactgcgtctgccctttgtagagcactcacccgagggaggaagacgttttcagttttgcttcctggtgaggctggaagttgagtgtaaggatgactgtgtaataagctcagcagcaggagttgtactgtgtcctttcatttgagcagagggctcttgcttgggaaggcagagaaggcccttcccaaacgctgggacagacctcctgtggaggcccgctgcctaaagcgtggagtcttcagtcatgaatgttggacgaagaaaccattggatgcttggtgggagagttgggagggcctggctctttgtctgaggagagccttaagtgttcatgtaaggaacagcttagcagcttctgatttcccacgtcccagcactttctctctgttttttattttatttttgagacttttgtatagcccaggccagcctcagtcactcagcagaggattcctgaccctctgggattatgtctcctgacattgggaacacatgtgtgcacacagcctgctctgctcttccttttcccatgttgagacgggctcctgctgtagtcctgcctggcctgagctggagtcggtcctgcctcctgaccgaccgtctggctgctttctaagctgggtagacttcccatactcactgttacagacagttacagcagagaagaaccatccttgtgtaagtctggttttgagagctcctgtctctgaggaagctgttggcaggagcagggatttctgttccttcttgtcagcccctagcattccactttgttttggcaggcgctggttctgctttgtaggtggaagtgaagctccactcagccacagactccagacagtgggtcacagagcagtctggaatgtgtagcagtcttatggatggtcacagcttttcttacatattcatattctatcaaacgttttgtaaagtataattttttatttatttttaatccccttagagaaaaagtaaaagtagagaagcctgggtattgtgtacacaacagaaaggacagtcaaggaaagggaagcagaaaggctcttatgtactctcagcttcatgcaacccctgtaactggccgcatatgtagggcatattttaattggttaaagcagtttgttgtggctgaggtcataagatatatacagttttgtatcctgatttttcactgaacttcattgttttttgtttgcttgcttttcccttcttctttgttttttgctttattgagatagggtttctctgtagcccaggctgtcctagaactccctttgtagaccaaacatctgattactggcctcacatggagagatctgtctctgcttttccagcgctgggattgaagtaggcctaatcttcatctaacttcttagcttgtcttttccatgttactacacacactgtgtaaataccagtgtgctaccctccttctttgagggtgactgttgggagtatttgtccaaatcctgggctccttgagtgcagactgatgctctaagatcagcaaaatgactgaactacatcctaacacttcccagtgtcctctcagtgtctcttcttatctgcagcattgcaggagtgaagtggccacagaccagtgtggaggcctgtgtgtgggaagtcagcaggtgccaagctattgaaacattggttttatttttttaaaccgatctctgtctctaaaagatgtgatgtttgtgttattcagctggaagtttgaatactgttctctgtgctgtccaacaagtgaagagttatgaaatgagccacttgtcctgggacatcctgtaatgagttcctcgggcttttctcctgtgagttttctgaaaaaagctgacctttttagtacaataaacggtgctaattgaaggaa

SEQ ID NO: 16 - DNA, 2807 bp, Rattus norvegicus
ccgcgttaaagccggacggagtacttctattgctggcagacctgccccggagcgcgtaaacccgcacgctcgcgccgcccacgtccagctcgcgaatccggaggcgtccggttccaataatgccccggaaggcagctagtccagaggaggcagaaaggatgccgggctctgaagagatagaggctgggcggccgcggccggttttacgctctgtgaactcgcgcgaaccctctcaggtcatcttctgcaaccgcagcccgcgcgtcgtgctgcctttgtggctcaactttgatggtgagcctcagccctacccgaccttaccaccgggcaccggccgccgcatccacagctaccgaggtcacctttggctcttcagggatgcggggacccatgatggacttctggttaaccaaacggaactgtttgtgccatccctcaatgttgatggacagcctatttttgccaacatcacattgccagtgtataccctgaaagagcggtgccttcaggttgtacggagcctggtcaagcctgagaactacaggaggctggacatcgtcaggtcgctctatgaagacttggaagaccacccaaatgtgcggaaagacatacagcggctgacccaagagcacctcgagaatcaggccctgggagaggagcctgaaggagtccactgagattacttggtcctgaatttcggccttgatggtctgaaatcgatctacacataggacaggtcactttctttcggttttaaaatggttcattcttggagtaaaatatccatcatgtaaaagaaagttaactaacatcactgagctttgtagtgtttaagactaaacctgcaaagtgccaccgagtctgccctttctagagcattcatcagaagaagctgttttcagttttgcttcctagtaaggctggatgttcagcataaggatgttagtgtgagcgattcgcgaggagttgctctgtgtcctctaatctgaagaatacagcctcgtatgaacagagccccaccgctcacttggggaaggctgagaaggccctaatgctgggatggacatcctgtagaggcccagtgcctaatgtgtgtggagtcttcagtcagtgttggaggaacaaaccattgggtacttagtgggagagttggagggcctggctctctgtctgaggaaatcttaagtgtacacgtaaaggaacaacttagcagcttctgtttagaaagaactttctcttgagtttccatgtctcagcactttctttctactttgttagtttattttattttatttttgagacttgtgtagcccaggtcagcctcagtcactccggaggggatgctggtcttgaattccggatcctctgggagtgtgtcagcattgggagcacatgggtcgaccacagtctgctctgttcttgcttttcccattttgagacgggctcatactgtagccctgcctggcctcaagctagagtcagccctgtctccccacccagtctggctgcttagactgggtgggcttcccatattcactgccccagacagcagagacgagctgtccttgtgtttaggtctggttttgagagttccttctctgaggaagctgctggcaggagcagggtttctgttccttcccgtcagcccctagcattccagctgggcgccgcactctgctttggcaggcgctagttatgcttcgatgggagaggggagtgaagctccctcagccacacactccaaaccatgggtcccagagcagtctgcaatgagtagtagtttgtgggcagtcacagcttttcttacatattcaaatattgatgttcactacttctatcaaacattttgtagaacataattttttatttgtttttaattcctcagcagagaaaaatgagagaagcttgggcattgtgtgtggcacacaccagaaaggacagtcagctgagagggtgcagaaaggctcttgtgcgctggctctcagctgcatgtagtcctgtaacccagtgttctctctagtacacattttaattggttaaagcactttgtcctggccgagatcataaggtatatacagttttgtggcctgatttttcattgaacttcattgttttgttttcccctctttttcgtttttggttttgtttttggtttgttttttgctttttgttttatcaagatagagtttctctgtgtagcccaggctgtcctggaactccctttgtagaccaaacaactgattactggcctcaaacggagatctgtctgcctctgcctctccagtgctgggattgaagtatgcctaatcttcatctaacttagtaacttgtcttttccatgttactgcacacactgtctaaattccggtgtgctagcctctccctttgagggtgactgttgggagtatttgtctcagtcctgggctccttgagtgcagtctgatgctctaagatcagcaaaatcactgaacttaatactgacactcctgccgccctctcagcgtctctccttagtctgcagcattccgtgagtgaagcggcacaggccagtgtggagcctgtgtgtgggaagtcagcaggtgccgagctattgcaacattggttttattttttaagcctatctctgtctctaaaagatgtaatgtttgtcttatttaactggaagtttgaatactgttctctgtgccttaggacatgagtcacttgtccttgcacatcctgtaagtgtgttcctcagactttgtactctcctttgagttctctgaaaaagctgacctttagtacaataaacggtgctaagtgaagga

SEQ ID NO: 17 - RNA, 21 nt, Artificial Sequence
  synthetic RNAi against pVHL
cggccuacggugcuggagguu

SEQ ID NO: 18 - RNA, 21 nt, Artificial Sequence
  synthetic RNAi against pVHL
gacuacggaggucgacucguu

SEQ ID NO: 19 - RNA, 21 nt, Artificial Sequence
  synthetic RNAi against pVHL
ugccccggagggcggagaauu

SEQ ID NO: 20 - RNA, 21 nt, Artificial Sequence
  synthetic RNAi against pVHL
cugggacgaggccgagguauu

SEQ ID NO: 21 - protein, 426 aa, Homo sapiens
MANDSGGPGGPSPSERDRQYCELCGKMENLLRCSRCRSSFYCCKEHQRQDWKKHKLVCQGSEGALGHGVGPHQHSGPAPPAAVPPPRAGAREPRKAAARRDNASGDAAKGKVKAKPPADPAAAASPCRAAAGGQGSAVAAEAEPGKEEPPARSSLFQEKANLYPPSNTPGDALSPGGGLRPNGQTKPLPALKLALEYIVPCMNKHGICVVDDFLGKETGQQIGDEVRALHDTGKFTDGQLVSQKSDSSKDIRGDKITWIEGKEPGCETIGLLMSSMDDLIRHCNGKLGSYKINGRTKAMVACYPGNGTGYVRHVDNPNGDGRCVTCIYYLNKDWDAKVSGGILRIFPEGKAQFADIEPKFDRLLFFWSDRRNPHEVQPAYATRYAITVWYFDADERARAKVKYLTGEKGVRVELNKPSDSVGKDVF

SEQ ID NO: 22 - protein, 407 aa, Homo sapiens
MDSPCQPQPLSQALPQLPGSSSEPLEPEPGRARMGVESYLPCPLLPSYHCPGVPSEASAGSGTPRATATSTTASPLRDGFGGQDGGELRPLQSEGAAALVTKGCQRLAAQGARPEAPKRKWAEDGGDAPSPSKRPWARQENQEAEREGGMSCSCSSGSGEASAGLMEEALPSAPERLALDYIVPCMRYYGICVKDSFLGAALGGRVLAEVEALKRGGRLRDGQLVSQRAIPPRSIRGDQIAWVEGHEPGCRSIGALMAHVDAVIRHCAGRLGSYVINGRTKAMVACYPGNGLGYVRHVDNPHGDGRCITCIYYLNQNWDVKVHGGLLQIFPEGRPVVANIEPLFDRLLIFWSDRRNPHEVKPAYATRYAITVWYFDAKERAAAKDKYQLASGQKGVQVPVSQPPTPT

SEQ ID NO: 23 - protein, 239 aa, Homo sapiens
MPLGHIMRLDLEKIALEYIVPCLHEVGFCYLDNFLGEVVGDCVLERVKQLHCTGALRDGQLAGPRAGVSKRHLRGDQITWIGGNEEGCEAISFLLSLIDRLVLYCGSRLGKYYVKERSKAMVACYPGNGTGYVRHVDNPNGDGRCITCIYYLNKNWDAKLHGGILRIFPEGKSFIADVEPIFDRLLFFWSDRRNPHEVQPSYATRYAMTVWYFDAEERAEAKKKFRNLTRKTESALTED

SEQ ID NO: 24 - DNA, 5163 bp, Homo sapiens
ttaggggcagaaaaacatttgtaataattaatggctttgagagacacaaggctttgtttgccccagagtattagttaacccacctagtgctcctaatcatacaatattaaggattgggagggacattcattgcctcactctctatttgtttcaccttctgtaaaattggtagaataatagtacccacttcatagcattgtatgatgattaaattggttaatatttttaaaatgcttagaacacagattgggcacataacagcaagcaccacatgtgtttataagataaattcctttgtgttgccttccgttaaagtttaaataagtaaataaataaataaatacttgcatgacattttgaagtctctctataacatctgagtaagtggcggctgcgacaatgctactggagttccagaatcgtgttggtgacaagattgttcaccagcatatggtgtggtgaaaactcactaatttggaattagttcagattattaagcctgaataggtgaaaatcctgaaatcaaggatctttggaactatttgaaatcagtattttatattttcctgttgtattcattaaagtgttgcaagtgttctatttgatggattaagtatatttaggatatacatgttcaatttgtgattttgtatacttaattggaacaagaaagctaataaaggttttgatatggacatctattcttttaagtaaacttcaatgaaaatatatgagtagagcatatagagatgtaaataatttgtggacacaccacagactgaaatagcaaatttaaaagaaattgttggaagaatcaagtgtttgtggaatgagtcctcctagtaaagttcctgctcttgtgaataattaagcctcatgtataattactatagcaaaaggaagcctaagaagtattagactctacttgtatttaaattacattttacataatttatgtgtatgaaaaatgttttaaatgcttattttcgtaagccatgagatagctcctttatattttaagaatttctgaattaatttgcttggattttattagtgcaaatggcagagctagcaattcctttttctgtgttcccattccatcctattcatccctcttttaggaaactctgaactctggattgtccttgtttacatacctgcctcctgcattggactatgtgtctctgagtgtagtatgactaattcatttgtttgtcaaggactctcaatgcatttgttgaacagcctaattagtaatgtctgcaacaatgacattttactgtatttaataaagctctgggaaagtaggatacacataagacaggtctaggtctaaattctttacagaaacttggatttttagttcggtttgaaatttgaagatgtgagtatatttatctcagtttcccaaaggacaagctaattggaattatcatcctctttcacttgattggatccccagaatgccatttacgcatgcagcaggattttataacagttttaaattctgtatatttgatgaagaggttttatatttttggattcaagcctctttttaaacttctacaatatggtttacaataattccttatatcctgcttttgaaatacatattacaactttttaagtttggaaggctatatttcaaggactgaagttacagtatactcaagtgatacacaagcctagcaccccactttccacatagtgttcgataaagattgataaactcgaaatcacagaccttttaattcttaagacaaatagcagcagaaagaaacatctttggcttatttctggtaaggtttttatgctctgtaaaacaaagaattgtattcatccgcgcagcacagattctattaaaaataaatgtgagagtcgttaatgtagtactgctcatttaccatcaaaattcacttttcaggaataatcccatcagtttaaattggatattggaatgagcattgattacatttaacttggtagcccaaaatttcttcatggggttttgaactcggcgggatttcaaaggttttaaaaatgagtttttgattttttttaaaaccctcaaatttcattacctttaaactaggtcgaaacggggcgcaagagattggattaacaccatagtaatacttattttgttcttaaccatttcagggcttcttgaaatagaggctgtatggtgtaatggaaaaaacagccttggaatctgggagcctgattcctggattcagtcccagttttgcgtgaccttgggcaagttactttacttctctgaatttccgtttcctcctctgcaaaatgaggatcgcaatagccaccttgcaaccttgactggagcgagcctcgcacaccccgcgccggcctggaggaagagcagccatgattacgccgccttcgctccgctacccgcttgcggctggcgccctcctccagcaggtgtaggcgctgccgcgctgccccacgcctttccgccgctcgcgggcctgcgcctcggcgtccccgaggaggccgctgcgggctgaggtagcgcaccggcctctcggcgtcccagtccggtcccgggcggagggaaagcgggcgacccacctccgaggcagaagccgaggcccggccccgccgagtgcggaggagcgcaggcagcccccgcccctcggccctccccccggccctcccggccctccctccgccccctccgccctcgcgcgccgcccgcccgggtcgccgcggggccgtggtgtacgtgcagagcgcgcagagcgagtggcgcccgtatgccctgcgctcctccacagcctgggccgggccgcccgggacgctgaggcggcggcggcggccgagggggccggtcttgcgctccccaggcccgcgcgcctgagcccaggttgccattcgccgcacaggccctattctctcagccctcggcggcgatgaggcgctgaggcggctgccggcgctgcgccggagcttaggactcggaagcggccgggccgagggcgtggggtgccggcctccctgaggcgagggtagcgggtgcatggcgcagtaacggcccctatctctctccccgctccccagcctcgggcgaggccgtccggccgctacccctcctgctcggccgccgcagtcgccgtcgccgccgccgccgccgccatggccaatgacagcggcgggcccggcgggccgagcccgagcgagcgagaccggcagtactgcgagctgtgcgggaagatggagaacctgctgcgctgcagccgctgccgcagctccttctactgctgcaaggagcaccagcgtcaggactggaagaagcacaagctcgtgtgccagggcagcgagggcgccctcggccacggagtgggcccacaccagcattccggccccgcgccgccggctgcagtgccgccgcccagggccggggcccgggagcccaggaaggcagcggcgcgccgggacaacgcctccggggacgcggccaagggaaaagtaaaggccaagcccccggccgacccagcggcggccgcgtcgccgtgtcgtgcggccgccggcggccagggctcggcggtggctgccgaagccgagcccggcaaggaggagccgccggcccgctcatcgctgttccaggagaaggcgaacctgtaccccccaagcaacacgcccggggatgcgctgagccccggcggcggcctgcggcccaacgggcagacgaagcccctgccggcgctgaagctggcgctcgagtacatcgtgccgtgcatgaacaagcacggcatctgtgtggtggacgacttcctcggcaaggagaccggacagcagatcggcgacgaggtgcgcgccctgcacgacaccgggaagttcacggacgggcagctggtcagccagaagagtgactcgtccaaggacatccgaggcgataagatcacctggatcgagggcaaggagcccggctgcgaaaccattgggctgctcatgagcagcatggacgacctgatacgccactgtaacgggaagctgggcagctacaaaatcaatggccggacgaaagccatggttgcttgttatccgggcaatggaacgggttatgtacgtcatgttgataatccaaatggagatggaagatgtgtgacatgtatatattatcttaataaagactgggatgccaaggtaagtggaggtatacttcgaatttttccagaaggcaaagcccagtttgctgacattgaacccaaatttgatagactgctgtttttctggtctgaccgtcgcaaccctcatgaagtacaaccagcatatgctacaaggtacgcaataactgtttggtattttgatgcagatgagagagcacgagctaaagtaaaatatctaacaggtgaaaaaggtgtgagggttgaactcaataaaccttcagattcggtcggtaaagacgtcttctagagcctttgatccagcaataccccacttcacctacaatattgttaactatttgttaacttgtgaatacgaataaatgggataaagaaaaatagacaaccagttcgcattttaataaggaaacagaaacaactttttgtgttgcatcaaacagaagattttgactgctgtgactttgtactgcatgatcaacttcaaatctgtgattgcttacaggaggaagataagctactaattgaaaatggtttttacatctggatatgaaataagtgccctgtgtagaatttttttcattcttatattttgccagatctgttatctagctgagttcatttcatctctcccttttttatatcaagtttgaatttgggataatttttctatattaggtacaatttatctaaactgaattgagaaaaaattacagtattattcctcaaaataacatcaatctatttttgtaaacctgttcatactattaaattttgccctaaaagacctcttaataatgattgttgccagtgactgatgattaattttattttacttaaaataagaaaaggagcactttaattacaactgaaaaatcagattgttttgcagtccttccttacactaatttgaactcttaaagattgctgctttttttttgacattgtcaataacgaaacctaattgtaaaacagtcaccatttactaccaataacttttagttaatgttttacaagg

SEQ ID NO: 25 - DNA, 2111 bp, Homo sapiens
gctttcccctgcctgcctgtctctagtttctctcacatccctttttttttttcctttctctagccaccctgaagggtcccttcccaagcccttagggaccgcagaggacttggggaccagcaagcaacccccagggcacgagaagagctcttgctgtctgccctgcctcaccctgccccacgccaggcccggtggcccccagctgcatcaagtggaggcggaggaggaggcggaggagggtggcaccatgggcccgggcggtgccctccatgcccgggggatgaagacactgctgccatggacagcccgtgccagccgcagcccctaagtcaggctctccctcagttaccagggtcttcgtcagagcccttggagcctgagcctggccgggccaggatgggagtggagagttacctgccctgtcccctgctcccctcctaccactgtccaggagtgcctagtgaggcctcggcagggagtgggacccccagagccacagccacctctaccactgccagccctcttcgggacggttttggcgggcaggatggtggtgagctgcggccgctgcagagtgaaggcgctgcagcgctggtcaccaaggggtgccagcgattggcagcccagggcgcacggcctgaggcccccaaacggaaatgggccgaggatggtggggatgccccttcacccagcaaacggccctgggccaggcaagagaaccaggaggcagagcgggagggtggcatgagctgcagctgcagcagtggcagtggtgaggccagtgctgggctgatggaggaggcgctgccctctgcgcccgagcgcctggccctggactatatcgtgccctgcatgcggtactacggcatctgcgtcaaggacagcttcctgggggcagcactgggcggtcgcgtgctggccgaggtggaggccctcaaacggggtgggcgcctgcgagacgggcagctagtgagccagagggcgatcccgccgcgcagcatccgtggggaccagattgcctgggtggaaggccatgaaccaggctgtcgaagcattggtgccctcatggcccatgtggacgccgtcatccgccactgcgcagggcggctgggcagctatgtcatcaacgggcgcaccaaggccatggtggcgtgttacccaggcaacgggctcgggtacgtaaggcacgttgacaatccccacggcgatgggcgctgcatcacctgtatctattacctgaatcagaactgggacgttaaggtgcatggcggcctgctgcagatcttccctgagggccggcccgtggtagccaacatcgagccactctttgaccggttgctcattttctggtctgaccggcggaacccccacgaggtgaagccagcctatgccaccaggtacgccatcactgtctggtattttgatgccaaggagcgggcagcagccaaagacaagtatcagctagcatcaggacagaaaggtgtccaagtacctgtatcacagccgcctacgcccacctagtggccagtcccagagccgcatggcagacagcttaaatgacttcaggagagccctgggcctgtgctggctgctccttccctgccaccgctgctgcttctgactttgcctctgtcctgcctggtgtggagggctctgtctgttgctgaggaccaaggaggagaagagacctttgctgccccatcatgggggctggggttgtcacctggacagggggcagccgtggaggccaccgttaccaactgaagctgggggcctgggtcctaccctgtctggtcatgaccccattaggtatggagagctgggaggaggcattgtcacttcccaccaggatgcaggacttggggttgaggtgagtcatggcctcttgctggcaatggggtgggaggagtacccccaagtcctctcactcctccagcctggaatgtgaagtgactccccaacccctttggccatggcaggcaccttttggactgggctgccactgcttgggcagagtaaaaggtgccaggaggagcatgggtgtggaagtcctgtcagccaagaaataaaagtttacctcagagctgcaaaaaaaaaaaaaaaaaaaaaaa

SEQ ID NO: 26 - DNA, 2722 bp, Homo sapiens
gagtctggccgcagtcgcggcagtggtggcttcccatccccaaaaggcgccctccgactccttgcgccgcactgctcgccgggccagtccggaaacgggtcgtggagctccgcaccactcccgctggttcccgaaggcagatcccttctcccgagagttgcgagaaactttcccttgtccccgacgctgcagcggctcgggtaccgtggcagccgcaggtttctgaaccccgggccacgctccccgcgcctcggcttcgcgctcgtgtagatcgttccctctctggttgcacgctggggatcccggacctcgattctgcgggcgagatgcccctgggacacatcatgaggctggacctggagaaaattgccctggagtacatcgtgccctgtctgcacgaggtgggcttctgctacctggacaacttcctgggcgaggtggtgggcgactgcgtcctggagcgcgtcaagcagctgcactgcaccggggccctgcgggacggccagctggcggggccgcgcgccggcgtctccaagcgacacctgcggggcgaccagatcacgtggatcgggggcaacgaggagggctgcgaggccatcagcttcctcctgtccctcatcgacaggctggtcctctactgcgggagccggctgggcaaatactacgtcaaggagaggtctaaggcaatggtggcttgctatccgggaaatggaacaggttatgttcgccacgtggacaaccccaacggtgatggtcgctgcatcacctgcatctactatctgaacaagaattgggatgccaagctacatggtgggatcctgcggatatttccagaggggaaatcattcatagcagatgtggagcccatttttgacagactcctgttcttctggtcagatcgtaggaacccacacgaagtgcagccctcttacgcaaccagatatgctatgactgtctggtactttgatgctgaagaaagggcagaagccaaaaagaaattcaggaatttaactaggaaaactgaatctgccctcactgaagactgaccgtgctctgaaatctgctggccttgttcattttagtaacggttcctgaattctcttaaattctttgagatccaaagatggcctcttcagtgacaacaatctccctgctacttcttgcatccttcacatccctgtcttgtgtgtggtacttcatgttttcttgccaagactgtgttgatcttcagatactctctttgccagatgaagttacttgctaactccagaaattcctgcagacatcctactcggccagcggtttacctgatagattcggtaatactatcaagagaagagcctaggagcacagcgagggaatgaaccttacttgcactttatgtatacttcctgatttgaaaggaggaggtttgaaaagaaaaaaatggaggtggtagatgccacagagaggcatcacggaagccttaacagcaggaaacagagaaatttgtgtcatctgaacaatttccagatgttcttaatccagggctgttggggtttctggagaattatcacaacctaatgacattaatacctctagaaagggctgctgtcatagtgaacaatttataagtgtcccatggggcagacactccttttttcccagtcctgcaacctggattttctgcctcagccccattttgctgaaaataatgactttctgaataaagatggcaacacaattttttctccattttcagttcttacctgggaacctaattccccagaagctaaaaaactagacattagttgttttggttgctttgttggaatggaatttaaatttaaatgaaaggaaaaatatatccctggtagttttgtgttaaccactgataactgtggaaagagctaggtctactgatatacaataaacatgtgtgcatcttgaacaatttgagaggggaggtggagttggaaatgtgggtgttcctgttttttttttttttttttttttagttttcctttttaatgagctcaccctttaacacaaaaaaagcaaggtgatgtattttaaaaaaggaagtggaaataaaaaaatctcaaagctatttgagttctcgtctgtccctagcagtctttcttcagctcacttggctctctagatccactgtggttggcagtatgaccagaatcatggaatttgctagaactgtggaagcttctactcctgcagtaagcacagatcgcactgcctcaataacttggtattgagcacgtattttgcaaaagctacttttcctagttttcagtattactttcatgttttaaaaatccctttaatttcttgcttgaaaatcccatgaacattaaagagccagaaatattttcctttgttatgtacggatatatatatatatagtcttccaagatagaagtttactttttcctcttctggttttggaaaatttccagataagacatgtcaccattaattctcaacgactgctctattttgttgtacggtaatagttatcaccttctaaattactatgtaatttattcacttattatgtttattgtcttgtatcctttctctggagtgtaagcacaatgaagacaggaattttgtatatttttaaccaatgcaacatactctcagcacctaaaatagtgccgggaacatagtaagggctcagtaaatacttgttgaataaactcagtctcctacattagcattctaa